SEQUENCE LISTING (242 sequences)

SEQ ID NO: 1 - DNA, 30 bp, Artificial Sequence
  splice donor region
ggggcggcgactggtgagtacgccaaaaat

SEQ ID NO: 2 - DNA, 30 bp, Artificial Sequence
  mutated splice donor region, MSD-2KO
ggggcggcgactgcagacaacgccaaaaat

SEQ ID NO: 3 - DNA, 8 bp, Artificial Sequence
  splice donor site consensus sequence
tggtragt

SEQ ID NO: 4 - DNA, 5 bp, Artificial Sequence
  major splice donor consensus sequence
ctggt

SEQ ID NO: 5 - DNA, 6 bp, Artificial Sequence
  splice donor region
cagaca

SEQ ID NO: 6 - DNA, 19 bp, Artificial Sequence
  mutated splice donor region
ggcgactgcagacaacgcc

SEQ ID NO: 7 - DNA, 9 bp, Artificial Sequence
  mutated splice donor region
gtggagact

SEQ ID NO: 8 - DNA, 19 bp, Artificial Sequence
  mutated splice donor region
ggcgagtggagactacgcc

SEQ ID NO: 9 - DNA, 19 bp, Artificial Sequence
  splice donor region
ggcgactggtgagtacgcc

SEQ ID NO: 10 - DNA, 5 bp, Artificial Sequence
  cryptic splice donor site consensus sequence
tgagt

SEQ ID NO: 11 - DNA, 30 bp, Artificial Sequence
  mutated splice donor region, MSD-2KOv2
ggggcggcgagtggagactacgccaaaaat

SEQ ID NO: 12 - DNA, 32 bp, Artificial Sequence
  mutated splice donor region, MSD-2KOm5
ggggaaggcaacagataaatatgccttaaaat

SEQ ID NO: 13 - DNA, 7 bp, Artificial Sequence
  major splice donor and cryptic splice donor region core sequence
gtgagta

SEQ ID NO: 14 - DNA, 23 bp, Artificial Sequence
  mutated splice donor region
aaggcaacagataaatatgcctt

SEQ ID NO: 15 - DNA, 642 bp, Artificial Sequence
  256U1 snRNA sequence
taaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgctgcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcccaagatctcatttgccgtgcgcgcttgcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctggtttcaaaagtagactgtacgctaagggtcatatctttttttgttttggtttgtgtcttggttggcgtcttaaatgttaa

SEQ ID NO: 16 - DNA, 15 bp, Artificial Sequence
  HIV-1 target sequence
gaccagatctgagcc

SEQ ID NO: 17 - DNA, 17 bp, Artificial Sequence
  U1 snRNA target-annealing sequence
atggctcagatctggtc

SEQ ID NO: 18 - DNA, 15 bp, Artificial Sequence
  HIV-1 target sequence
tgggagctctctggc

SEQ ID NO: 19 - DNA, 17 bp, Artificial Sequence
  U1 snRNA target-annealing sequence
atgccagagagctccca

SEQ ID NO: 20 - DNA, 15 bp, Artificial Sequence
  HIV-1 target sequence
taaagcttgccttga

SEQ ID NO: 21 - DNA, 17 bp, Artificial Sequence
  U1 snRNA target-annealing sequence
attcaaggcaagcttta

SEQ ID NO: 22 - DNA, 15 bp, Artificial Sequence
  HIV-1 target sequence
tagagatccctcaga

SEQ ID NO: 23 - DNA, 17 bp, Artificial Sequence
  U1 snRNA target-annealing sequence
attctgagggatctcta

SEQ ID NO: 24 - DNA, 9 bp, Artificial Sequence
  HIV-1 target sequence
gcagtggcg

SEQ ID NO: 25 - DNA, 11 bp, Artificial Sequence
  U1 snRNA target-annealing sequence
atcgccactgc

SEQ ID NO: 26 - DNA, 15 bp, Artificial Sequence
  HIV-1 target sequence
agtggcgcccgaaca

SEQ ID NO: 27 - DNA, 17 bp, Artificial Sequence
  U1 snRNA target-annealing sequence
attgttcgggcgccact

SEQ ID NO: 28 - DNA, 15 bp, Artificial Sequence
  HIV-1 target sequence
gggacttgaaagcga

SEQ ID NO: 29 - DNA, 17 bp, Artificial Sequence
  U1 snRNA target-annealing sequence
attcgctttcaagtccc

SEQ ID NO: 30 - DNA, 15 bp, Artificial Sequence
  HIV-1 target sequence
aagggaaaccagagg

SEQ ID NO: 31 - DNA, 17 bp, Artificial Sequence
  U1 snRNA target-annealing sequence
atcctctggtttccctt

SEQ ID NO: 32 - DNA, 15 bp, Artificial Sequence
  HIV-1 target sequence
ggactcggcttgctg

SEQ ID NO: 33 - DNA, 17 bp, Artificial Sequence
  U1 snRNA target-annealing sequence
atcagcaagccgagtcc

SEQ ID NO: 34 - DNA, 15 bp, Artificial Sequence
  HIV-1 target sequence
aagcgcgcacggcaa

SEQ ID NO: 35 - DNA, 17 bp, Artificial Sequence
  U1 snRNA target-annealing sequence
atttgccgtgcgcgctt

SEQ ID NO: 36 - DNA, 15 bp, Artificial Sequence
  HIV-1 target sequence
gaggcgaggggcggc

SEQ ID NO: 37 - DNA, 17 bp, Artificial Sequence
  U1 snRNA target-annealing sequence
atgccgcccctcgcctc

SEQ ID NO: 38 - DNA, 15 bp, Artificial Sequence
  HIV-1 target sequence
gactggtgagtacgc

SEQ ID NO: 39 - DNA, 17 bp, Artificial Sequence
  U1 snRNA target-annealing sequence
atgcgtactcaccagtc

SEQ ID NO: 40 - DNA, 11 bp, Artificial Sequence
  HIV-1 target sequence
aattttgacta

SEQ ID NO: 41 - DNA, 11 bp, Artificial Sequence
  U1 snRNA target-annealing sequence
atgtcaaaatt

SEQ ID NO: 42 - DNA, 15 bp, Artificial Sequence
  HIV-1 target sequence
aattttgactagcgg

SEQ ID NO: 43 - DNA, 17 bp, Artificial Sequence
  U1 snRNA target-annealing sequence
atccgctagtcaaaatt

SEQ ID NO: 44 - DNA, 15 bp, Artificial Sequence
  HIV-1 target sequence
gcggaggctagaagg

SEQ ID NO: 45 - DNA, 17 bp, Artificial Sequence
  U1 snRNA target-annealing sequence
atccttctagcctccgc

SEQ ID NO: 46 - DNA, 15 bp, Artificial Sequence
  HIV-1 target sequence
agagagatgggtgcg

SEQ ID NO: 47 - DNA, 17 bp, Artificial Sequence
  U1 snRNA target-annealing sequence
atcgcacccatctctct

SEQ ID NO: 48 - DNA, 15 bp, Artificial Sequence
  HIV-1 target sequence
agagcgtcggtatta

SEQ ID NO: 49 - DNA, 17 bp, Artificial Sequence
  U1 snRNA target-annealing sequence
attaatactgacgctct

SEQ ID NO: 50 - DNA, 15 bp, Artificial Sequence
  HIV-1 target sequence
agcgggggagaatta

SEQ ID NO: 51 - DNA, 17 bp, Artificial Sequence
  U1 snRNA target-annealing sequence
attaattctcccccgct

SEQ ID NO: 52 - DNA, 15 bp, Artificial Sequence
  HIV-1 target sequence
gatcgcgatgggaaa

SEQ ID NO: 53 - DNA, 17 bp, Artificial Sequence
  U1 snRNA target-annealing sequence
attttcccatcgcgatc

SEQ ID NO: 54 - DNA, 15 bp, Artificial Sequence
  HIV-1 target sequence
aaattcggttaaggc

SEQ ID NO: 55 - DNA, 17 bp, Artificial Sequence
  U1 snRNA target-annealing sequence
atgccttaaccgaattt

SEQ ID NO: 56 - DNA, 15 bp, Artificial Sequence
  HIV-1 target sequence
gatcttcagacctgg

SEQ ID NO: 57 - DNA, 17 bp, Artificial Sequence
  U1 snRNA target-annealing sequence
atccaggtctgaagatc

SEQ ID NO: 58 - DNA, 15 bp, Artificial Sequence
  HIV-1 target sequence
ttacacaagcttaat

SEQ ID NO: 59 - DNA, 17 bp, Artificial Sequence
  U1 snRNA target-annealing sequence
atattaagcttgtgtaa

SEQ ID NO: 60 - DNA, 15 bp, Artificial Sequence
  HIV-1 target sequence
tagtagacataatag

SEQ ID NO: 61 - DNA, 17 bp, Artificial Sequence
  U1 snRNA target-annealing sequence
atctattatgtctacta

SEQ ID NO: 62 - DNA, 9 bp, Artificial Sequence
  Control U1 snRNA target sequence
ctacaggaa

SEQ ID NO: 63 - DNA, 11 bp, Artificial Sequence
  U1 snRNA target-annealing sequence
atttcctgtag

SEQ ID NO: 64 - DNA, 9 bp, Artificial Sequence
  Control U1 snRNA target sequence
tcatctgtg

SEQ ID NO: 65 - DNA, 11 bp, Artificial Sequence
  U1 snRNA target-annealing sequence
atcacagatga

SEQ ID NO: 66 - DNA, 153 bp, Artificial Sequence
  modified U1 snRNA sequence (main U1 snRNA sequence [clover leaf] (nt 410-562) of the U1_256 sequence)
gcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctg

SEQ ID NO: 67 - DNA, 15 bp, Artificial Sequence
  HIV-1 target sequence
agctctctcgacgca

SEQ ID NO: 68 - DNA, 17 bp, Artificial Sequence
  U1 snRNA target-annealing sequence
attgcgtcgagagagct

SEQ ID NO: 69 - DNA, 7 bp, Artificial Sequence
  3′ tbs-Kozak junction variant
gagaatg

SEQ ID NO: 70 - DNA, 7 bp, Artificial Sequence
  3′ tbs-Kozak junction variant
gagcatg

SEQ ID NO: 71 - DNA, 7 bp, Artificial Sequence
  3′ tbs-Kozak junction variant
gaggatg

SEQ ID NO: 72 - DNA, 7 bp, Artificial Sequence
  3′ tbs-Kozak junction variant
tagaatg

SEQ ID NO: 73 - DNA, 7 bp, Artificial Sequence
  3′ tbs-Kozak junction variant
tagcatg

SEQ ID NO: 74 - DNA, 7 bp, Artificial Sequence
  3′ tbs-Kozak junction variant
taggatg

SEQ ID NO: 75 - DNA, 8 bp, Artificial Sequence
  3′ tbs-Kozak junction variant
gagaaatg

SEQ ID NO: 76 - DNA, 8 bp, Artificial Sequence
  3′ tbs-Kozak junction variant
gagacatg

SEQ ID NO: 77 - DNA, 8 bp, Artificial Sequence
  3′ tbs-Kozak junction variant
gagagatg

SEQ ID NO: 78 - DNA, 8 bp, Artificial Sequence
  3′ tbs-Kozak junction variant
gagcaatg

SEQ ID NO: 79 - DNA, 8 bp, Artificial Sequence
  3′ tbs-Kozak junction variant
gagccatg

SEQ ID NO: 80 - DNA, 8 bp, Artificial Sequence
  3′ tbs-Kozak junction variant
gagcgatg

SEQ ID NO: 81 - DNA, 8 bp, Artificial Sequence
  3′ tbs-Kozak junction variant
gaggaatg

SEQ ID NO: 82 - DNA, 8 bp, Artificial Sequence
  3′ tbs-Kozak junction variant
gaggcatg

SEQ ID NO: 83 - DNA, 8 bp, Artificial Sequence
  3′ tbs-Kozak junction variant
gagggatg

SEQ ID NO: 84 - DNA, 8 bp, Artificial Sequence
  3′ tbs-Kozak junction variant
tagaaatg

SEQ ID NO: 85 - DNA, 8 bp, Artificial Sequence
  3′ tbs-Kozak junction variant
tagacatg

SEQ ID NO: 86 - DNA, 8 bp, Artificial Sequence
  3′ tbs-Kozak junction variant
tagagatg

SEQ ID NO: 87 - DNA, 8 bp, Artificial Sequence
  3′ tbs-Kozak junction variant
tagcaatg

SEQ ID NO: 88 - DNA, 8 bp, Artificial Sequence
  3′ tbs-Kozak junction variant
tagccatg

SEQ ID NO: 89 - DNA, 8 bp, Artificial Sequence
  3′ tbs-Kozak junction variant
tagcgatg

SEQ ID NO: 90 - DNA, 8 bp, Artificial Sequence
  3′ tbs-Kozak junction variant
taggaatg

SEQ ID NO: 91 - DNA, 8 bp, Artificial Sequence
  3′ tbs-Kozak junction variant
taggcatg

SEQ ID NO: 92 - DNA, 8 bp, Artificial Sequence
  3′ tbs-Kozak junction variant
tagggatg

SEQ ID NO: 93 - DNA, 45 bp, Artificial Sequence
  L33 improved leader derived from a splicing event
ctttttcgcaacgggtttgccgccagaacacaggtgtcgtgaaaa

SEQ ID NO: 94 - protein, 75 aa, Bacillus subtilis
MNQKHSSDFVVIKAVEDGVNVIGLTRGTDTKFHHSEKLDKGEVIIAQFTEHTSAIKVRGEALIQTAYGEMKSEKK

SEQ ID NO: 95 - protein, 86 aa, Aminomonas paucivorans
MKEGEEAKTSVLSDYVVVKALENGVTVIGLTRGQETKFAHTEKLDDGEVWIAQFTEHTSAIKVRGASEIHTKHGMLFSGRGRNEKG

SEQ ID NO: 96 - protein, 81 aa, Desulfotomaculum hydrothermale
MNPMTDRSDITGDYVVVKALENGVTIIGLTRGGVTKFHHTEKLDKGEIMIAQFTEHTSAIKIRGRAELLTKHGKIRTEVDS

SEQ ID NO: 97 - protein, 74 aa, Bacillus stearothermophilus
MYTNSDFVVIKALEDGVNVIGLTRGADTRFHHSEKLDKGEVLIAQFTEHTSAIKVRGKAYIQTRHGVIESEGKK

SEQ ID NO: 98 - protein, 74 aa, Bacillus stearothermophilus
MYTNSDFVVIKALEDGVNVIGLTRGADTRFHHSEKLDKGEVLIAQFTEHTSAIKVRGKAYIQTRHGVIENEGKK

SEQ ID NO: 99 - protein, 76 aa, Bacillus halodurans
MNVGDNSNFFVIKAKENGVNVFGMTRGTDTRFHHSEKLDKGEVMIAQFTEHTSAVKIRGKAIIQTSYGTLDTEKDE

SEQ ID NO: 100 - protein, 80 aa, Carboxydothermus hydrogenoformans
MVCDNFAFSSAINAEYIVVKALENGVTIMGLTRGKDTKFHHTEKLDKGEVMVAQFTEHTSAIKIRGKAEIYTKHGVIKNE

SEQ ID NO: 101 - DNA, 33 bp, Artificial Sequence
  L33 improved leader
ctttttcgcaacgggtttgccgccagaacacag

SEQ ID NO: 102 - DNA, 12 bp, Artificial Sequence
  L12 improved leader
ctttttcgcaac

SEQ ID NO: 103 - DNA, 9 bp, Artificial Sequence
  extended Kozak consensus sequence
  misc_feature (2)..(3): n is a, c, g, or t
gnnrvvatg

SEQ ID NO: 104 - DNA, 6 bp, Artificial Sequence
  core Kozak consensus sequence
rvvatg

SEQ ID NO: 105 - DNA, 32 bp, Artificial Sequence
  EMCV M loops
cgtggttttcctttgaaaaacacgatgatacc

SEQ ID NO: 106 - DNA, 56 bp, Artificial Sequence
  optimal (overlapping) tbs-Kozak
gagtttagcggagtggagaagagcggagccgagcctagcagagacgagccgagatg

SEQ ID NO: 107 - DNA, 65 bp, Artificial Sequence
  optimal (overlapping) tbs-MCS-Kozak
gagtttagcggagtggagaagagcggagccgagcctagcagagacgagaagagctctagaccatg

SEQ ID NO: 108 - DNA, 6 bp, Artificial Sequence
  3′ tbs-Kozak junction variant
tagatg

SEQ ID NO: 109 - DNA, 8 bp, Artificial Sequence
  3′ tbs-Kozak junction variant
gagatatg

SEQ ID NO: 110 - DNA, 8 bp, Artificial Sequence
  3′ tbs-Kozak junction variant
gagctatg

SEQ ID NO: 111 - DNA, 8 bp, Artificial Sequence
  3′ tbs-Kozak junction variant
tagatatg

SEQ ID NO: 112 - DNA, 8 bp, Artificial Sequence
  3′ tbs-Kozak junction variant
tagctatg

SEQ ID NO: 113 - DNA, 6 bp, Artificial Sequence
  3′ tbs-Kozak consensus sequence
  misc_feature (4)..(5): n is a, c, g, or t
kagnng

SEQ ID NO: 114 - DNA, 6 bp, Artificial Sequence
  3′ tbs-Kozak consensus sequence
kagatg

SEQ ID NO: 115 - DNA, 7 bp, Artificial Sequence
  3′ tbs-Kozak consensus sequence
  misc_feature (4)..(5): n is a, c, g, or t
kagnntg

SEQ ID NO: 116 - DNA, 7 bp, Artificial Sequence
  3′ tbs-Kozak consensus sequence
  misc_feature (4)..(4): n is a, c, g, or t
kagnatg

SEQ ID NO: 117 - DNA, 1082 bp, Artificial Sequence
  Chicken beta-Actin / Rabbit beta-globin chimeric 5′UTR-intron with tbs-kzkV0.G variant
cggcgggcgggaacgttgccttcgccccgtgccccgctccgcgccgcctcgcgccgcccgccccggctctgactgaccgcgttactcccacaggtgagcgggcgggacggcccttctccctccgggctgtaattagcgcttggtttaatgacggctcgtttcttttctgtggctgcgtgaaagccttaaagggctccgggagggcctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcgggccgggggcggtgccccgcggtgcgggggggctgcgaggggaacaaaggctgcgtgcggggtgtgtgcgtgggggggtgagcagggggtgtgggcgcggcggtcgggctgtaacccccccctggcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctttagagcctctgctaaccatgttcatgccttcttctttttcctacagctcctgggcaaagagtttagcggagtggagaagagcggagccgagcctagcagagacgagccgagatg

SEQ ID NO: 118 - DNA, 1040 bp, Artificial Sequence
  EF1a 5′UTR-intron with tbskzkV0.G variant
ctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagagtttagcggagtggagaagagcggagccgagcctagcagagacgagccgagatg

SEQ ID NO: 119 - DNA, 161 bp, Artificial Sequence
  spliced sequence corresponding to SEQ ID NO 117
cggcgggcgggaacgttgccttcgccccgtgccccgctccgcgccgcctcgcgccgcccgccccggctctgactgaccgcgttactcccacagctcctgggcaaagagtttagcggagtggagaagagcggagccgagcctagcagagacgagccgagatg

SEQ ID NO: 120 - DNA, 101 bp, Artificial Sequence
  spliced sequence corresponding to SEQ ID NO 118
ctttttcgcaacgggtttgccgccagaacacaggtgtcgtgaaaagagtttagcggagtggagaagagcggagccgagcctagcagagacgagccgagatg

SEQ ID NO: 121 - DNA, 1026 bp, Artificial Sequence
  Chicken beta-Actin / Rabbit beta-globin chimeric 5′UTR-intron
cggcgggcgggaacgttgccttcgccccgtgccccgctccgcgccgcctcgcgccgcccgccccggctctgactgaccgcgttactcccacaggtgagcgggcgggacggcccttctccctccgggctgtaattagcgcttggtttaatgacggctcgtttcttttctgtggctgcgtgaaagccttaaagggctccgggagggcctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcgggccgggggcggtgccccgcggtgcgggggggctgcgaggggaacaaaggctgcgtgcggggtgtgtgcgtgggggggtgagcagggggtgtgggcgcggcggtcgggctgtaacccccccctggcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctttagagcctctgctaaccatgttcatgccttcttctttttcctacagctcctgggcaaa

SEQ ID NO: 122 - DNA, 984 bp, Artificial Sequence
  EF1a 5′UTR-intron
ctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaa

SEQ ID NO: 123 - DNA, 1030 bp, Artificial Sequence
  Chicken beta-Actin / Rabbit beta-globin chimeric 5′UTR-intron-tbs consensus
  misc_feature (1027)..(1030): kagn(2-3) may be repeated 10-11 times
  misc_feature (1030)..(1030): n may be repeated 2-3 times
cggcgggcgggaacgttgccttcgccccgtgccccgctccgcgccgcctcgcgccgcccgccccggctctgactgaccgcgttactcccacaggtgagcgggcgggacggcccttctccctccgggctgtaattagcgcttggtttaatgacggctcgtttcttttctgtggctgcgtgaaagccttaaagggctccgggagggcctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcgggccgggggcggtgccccgcggtgcgggggggctgcgaggggaacaaaggctgcgtgcggggtgtgtgcgtgggggggtgagcagggggtgtgggcgcggcggtcgggctgtaacccccccctggcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctttagagcctctgctaaccatgttcatgccttcttctttttcctacagctcctgggcaaaksgn

SEQ ID NO: 124 - DNA, 988 bp, Artificial Sequence
  EF1a 5′UTR-intron-tbs consensus
  misc_feature (985)..(988): kagn(2-3) may be repeated 10-11 times
  misc_feature (988)..(988): n may be repeated 2-3 times
ctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaakagn

SEQ ID NO: 125 - RNA, 55 nt, Artificial Sequence
  TRAP binding site variant - [KAGNN]11
gaguuuagcggaguggagaagagcggagccgagccuagcagagacgaguggagcu

SEQ ID NO: 126 - RNA, 55 nt, Artificial Sequence
  TRAP binding site variant - [KAGNN]11
gaguuuagcggaguggagaagagcggagccgagccuagcagagacgagaagagcu

SEQ ID NO: 127 - RNA, 30 nt, Artificial Sequence
  TRAP binding site variant - [KAGNN]6
uaguuuaguuuaguuuaguuuaguuuaguu

SEQ ID NO: 128 - RNA, 30 nt, Artificial Sequence
  TRAP binding site variant - [KAGNN]6
uaguuuaguugaguuuaguugaguuuaguu

SEQ ID NO: 129 - RNA, 32 nt, Artificial Sequence
  TRAP binding site variant - [KAGNN]6
gaguuugaguugaguugaguuugaguugaguu

SEQ ID NO: 130 - RNA, 32 nt, Artificial Sequence
  TRAP binding site variant - [KAGNN]6
uaguuugaguuuaguugaguuuuaguugaguu

SEQ ID NO: 131 - DNA, 55 bp, Artificial Sequence
  TRAP binding site variant - [KAGNN]11
gagtttagcggagtggagaagagcggagccgagcctagcagagacgagtggagct

SEQ ID NO: 132 - DNA, 50 bp, Artificial Sequence
  TRAP binding site variant - [KAGNN]10
gagtttagcggagtggagaagagcggagccgagcctagcagagacgagaa

SEQ ID NO: 133 - DNA, 45 bp, Artificial Sequence
  TRAP binding site variant - [KAGNN]9
gagtttagcggagtggagaagagcggagccgagcctagcagagac

SEQ ID NO: 134 - DNA, 40 bp, Artificial Sequence
  TRAP binding site variant - [KAGNN]8
gagtttagcggagtggagaagagcggagccgagcctagca

SEQ ID NO: 135 - DNA, 35 bp, Artificial Sequence
  TRAP binding site variant - [KAGNN]7
gagtttagcggagtggagaagagcggagccgagcc

SEQ ID NO: 136 - DNA, 30 bp, Artificial Sequence
  TRAP binding site variant - [KAGNN]6
gagtttagcggagtggagaagagcggagcc

SEQ ID NO: 137 - DNA, 58 bp, Artificial Sequence
  TRAP binding site variant - [KAGNNN]3[KAGNN]8
gagtttagcggagtggagaaagagacggagccgagacctagcagagacgagaagagct

SEQ ID NO: 138 - DNA, 56 bp, Artificial Sequence
  TRAP binding site variant - [KAGNNN]1[KAGNN]10
gagtttagcggagtggagaagagacggagccgagcctagcagagacgagaagagct

SEQ ID NO: 139 - DNA, 55 bp, Artificial Sequence
  TRAP binding site variant - [KAGNN]11
gagtttagcggagtggagaagagcggagccgagcctagcagagacgagaagagct

SEQ ID NO: 140 - DNA, 41 bp, Artificial Sequence
  TRAP binding site variant - [KAGNNN]1[KAGNN]7
gagtttagcggagtggagaaagagcggagccgagcctagca

SEQ ID NO: 141 - DNA, 36 bp, Artificial Sequence
  TRAP binding site variant - [KAGNNN]1[KAGNN]6
gagtttagcggagtggagaaagagcggagccgagcc

SEQ ID NO: 142 - DNA, 6 bp, Artificial Sequence
  3′ tbs-Kozak junction variant
gagatg

SEQ ID NO: 143 - DNA, 7 bp, Artificial Sequence
  3′ tbs-Kozak junction variant
kagvatg

SEQ ID NO: 144 - DNA, 8 bp, Artificial Sequence
  3′ tbs-Kozak junction variant
kagvvatg

SEQ ID NO: 145 - DNA, 9 bp, Artificial Sequence
  3′ tbs-Kozak junction variant
kagrvvatg

SEQ ID NO: 146 - DNA, 10 bp, Artificial Sequence
  3′ tbs-Kozak junction variant
  misc_feature (4)..(4): n is a, c, g, or t
kagnrvvatg

SEQ ID NO: 147 - DNA, 11 bp, Artificial Sequence
  optimal 3′ tbs-Kozak junction variant
kagccgagatg

SEQ ID NO: 148 - DNA, 13 bp, Artificial Sequence
  optimal 3′ tbs-Kozak junction variant
  misc_feature (4)..(4): n is a, c, g, or t
kagnggagccatg

SEQ ID NO: 149 - DNA, 14 bp, Artificial Sequence
  optimal 3′ tbs-Kozak junction variant
  misc_feature (4)..(5): n is a, c, g, or t
kagnngagaccatg

SEQ ID NO: 150 - DNA, 12 bp, Artificial Sequence
  3′ tbs-Kozak junction variant
kaggcgagcatg

SEQ ID NO: 151 - DNA, 15 bp, Artificial Sequence
  spacer variant
atagcagagacggct

SEQ ID NO: 152 - DNA, 10 bp, Artificial Sequence
  spacer variant
atagcagaga

SEQ ID NO: 153 - DNA, 5 bp, Artificial Sequence
  spacer variant
atagc

SEQ ID NO: 154 - DNA, 26 bp, Artificial Sequence
  spacer variant
atatcagagacggctagcgtatacca

SEQ ID NO: 155 - DNA, 15 bp, Artificial Sequence
  spacer variant
atatcagagacggct

SEQ ID NO: 156 - DNA, 10 bp, Artificial Sequence
  spacer variant
agagacggct

SEQ ID NO: 157 - DNA, 5 bp, Artificial Sequence
  spacer variant
tacca

SEQ ID NO: 158 - DNA, 15 bp, Artificial Sequence
  3′ tbs-MCS-Kozak variant
gagctctagavvatg

SEQ ID NO: 159 - DNA, 16 bp, Artificial Sequence
  3′ tbs-MCS-Kozak variant
gagctcgtcgacvatg

SEQ ID NO: 160 - DNA, 20 bp, Artificial Sequence
  3′ tbs-MCS-Kozak variant
gagctcgaattcgaavvatg

SEQ ID NO: 161 - DNA, 21 bp, Artificial Sequence
  3′ tbs-MCS-Kozak variant
gagctctagacgtcgacvatg

SEQ ID NO: 162 - DNA, 22 bp, Artificial Sequence
  3′ tbs-MCS-Kozak variant
gagctctagaattcgaavvatg

SEQ ID NO: 163 - DNA, 23 bp, Artificial Sequence
  3′ tbs-MCS-Kozak variant
gagctctagatatcgatrvvatg

SEQ ID NO: 164 - DNA, 25 bp, Artificial Sequence
  3′ tbs-MCS-Kozak variant
kagactagtacttaagcttrvvatg

SEQ ID NO: 165 - DNA, 15 bp, Artificial Sequence
  3′ tbs-MCS-Kozak variant
gagctctagaccatg

SEQ ID NO: 166 - DNA, 16 bp, Artificial Sequence
  3′ tbs-MCS-Kozak variant
gagctcgtcgaccatg

SEQ ID NO: 167 - DNA, 20 bp, Artificial Sequence
  3′ tbs-MCS-Kozak variant
gagctcgaattcgaaccatg

SEQ ID NO: 168 - DNA, 21 bp, Artificial Sequence
  3′ tbs-MCS-Kozak variant
gagctctagacgtcgaccatg

SEQ ID NO: 169 - DNA, 22 bp, Artificial Sequence
  3′ tbs-MCS-Kozak variant
gagctctagaattcgaaccatg

SEQ ID NO: 170 - DNA, 23 bp, Artificial Sequence
  3′ tbs-MCS-Kozak variant
gagctctagatatcgataccatg

SEQ ID NO: 171 - DNA, 25 bp, Artificial Sequence
  3′ tbs-MCS-Kozak variant
kagactagtacttaagcttaccatg

SEQ ID NO: 172 - DNA, 74 bp, Artificial Sequence
  illustrative nucleic acid sequence 1 containing L33 Improved leader, optimal (overlapping) tbs ([KAGNN]8)-Kozak junction
ctttttcgcaacgggtttgccgccagaacacaggagtttagcggagtggagaagagcggagccgagccgagatg

SEQ ID NO: 173 - DNA, 89 bp, Artificial Sequence
  illustrative nucleic acid sequence 2 containing L33 Improved leader, optimal (overlapping) tbs ([KAGNN]11)-Kozak junction
ctttttcgcaacgggtttgccgccagaacacaggagtttagcggagtggagaagagcggagccgagcctagcagagacgagccgagatg

SEQ ID NO: 174 - DNA, 68 bp, Artificial Sequence
  illustrative nucleic acid sequence 3 containing L12 Improved leader, optimal (overlapping) tbs ([KAGNN]11)-Kozak junction
ctttttcgcaacgagtttagcggagtggagaagagcggagccgagcctagcagagacgagccgagatg

SEQ ID NO: 175 - DNA, 101 bp, Artificial Sequence; identical to SEQ ID NO: 120
  illustrative nucleic acid sequence 4 for intron-containing 5′UTRs, resulting in a spliced leader comprising L33, optimal (overlapping) tbs ([KAGNN]11)-Kozak junction

SEQ ID NO: 176 - DNA, 56 bp, Artificial Sequence
  illustrative nucleic acid sequence 5 containing improved spacer, optimal (overlapping) tbs ([KAGNN]8)-Kozak junction
atagcagagacggctgagtttagcggagtggagaagagcggagccgagccgagatg

SEQ ID NO: 177 - DNA, 98 bp, Artificial Sequence
  illustrative nucleic acid sequence 6 containing L33 Improved leader tbs ([KAGNN]11)-MCS-Kozak
ctttttcgcaacgggtttgccgccagaacacaggagtttagcggagtggagaagagcggagccgagcctagcagagacgagaagagctctagaccatg

SEQ ID NO: 178 - DNA, 80 bp, Artificial Sequence
  illustrative nucleic acid sequence 7 containing improved spacer, tbs ([KAGNN]11)-MCS-Kozak
atagcagagacggctgagtttagcggagtggagaagagcggagccgagcctagcagagacgagaagagctctagaccatg

SEQ ID NO: 179 - DNA, 12 bp, Artificial Sequence
  tbs-Kozak region
acagccaccatg

SEQ ID NO: 180 - DNA, 17 bp, Artificial Sequence
  HpaI variant tbs-Kozak region
gagttaacgccaccatg

SEQ ID NO: 181 - DNA, 13 bp, Artificial Sequence
  consensus Kozak sequence (DNA)
gccgccrccatgg

SEQ ID NO: 182 - RNA, 13 nt, Artificial Sequence
  consensus Kozak sequence (RNA)
gccgccrccaugg

SEQ ID NO: 183 - RNA, 10 nt, Artificial Sequence
  extended Kozak consensus sequence
  misc_feature (2)..(3): n is a, c, g, or u
gnnrvvaugg

SEQ ID NO: 184 - DNA, 17 bp, Artificial Sequence
  original 3′ tbs-Kozak junction sequence
  misc_feature (4)..(5): n is a, c, g, or t
kagnnacagccaccatg

SEQ ID NO: 185 - DNA, 67 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
  misc_feature (54)..(55): n is a, c, g, or t
kagnnkagnnkagnnkagnnkagnnkagnnkagnnkagnnkagnnkagnnkagnnacagccaccatg

SEQ ID NO: 186 - DNA, 56 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagatg

SEQ ID NO: 187 - DNA, 56 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagatg

SEQ ID NO: 188 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagcatg

SEQ ID NO: 189 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagaatg

SEQ ID NO: 190 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngaggatg

SEQ ID NO: 191 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagaatg

SEQ ID NO: 192 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagcatg

SEQ ID NO: 193 - DNA, 57 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntaggatg

SEQ ID NO: 194 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagccatg

SEQ ID NO: 195 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagaaatg

SEQ ID NO: 196 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagagatg

SEQ ID NO: 197 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagacatg

SEQ ID NO: 198 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagatatg

SEQ ID NO: 199 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagcaatg

SEQ ID NO: 200 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagcgatg

SEQ ID NO: 201 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagctatg

SEQ ID NO: 202 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngaggaatg

SEQ ID NO: 203 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngagggatg

SEQ ID NO: 204 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnngaggcatg

SEQ ID NO: 205 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagaaatg

SEQ ID NO: 206 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagagatg

SEQ ID NO: 207 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagacatg

SEQ ID NO: 208 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagatatg

SEQ ID NO: 209 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagcaatg

SEQ ID NO: 210 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagcgatg

SEQ ID NO: 211 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagccatg

SEQ ID NO: 212 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagctatg

SEQ ID NO: 213 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntaggaatg

SEQ ID NO: 214 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntagggatg

SEQ ID NO: 215 - DNA, 58 bp, Artificial Sequence
  overlapping tbs 3′KAGNN-Kozak variant
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
kagnnkagnnkagnnkagnnkagnnkagnnkagnnkagnnkagnnkagnntaggcatg

SEQ ID NO: 216 - RNA, 164 nt, Artificial Sequence
  U1 snRNA molecule
auacuuaccuggcaggggagauaccaugaucacgaaggugguuuucccagggcgaggcuuauccauugcacuccggaugugcugaccccugcgauuuccccaaaugugggaaacucuacugcauaauuugugguagugggggacugcguucgcgcuuuccccug

SEQ ID NO: 217 - DNA, 13 bp, Artificial Sequence
  mutated splice donor (MSD) region, deltaSL2
ggggcgcaaaaat

SEQ ID NO: 218 - DNA, 55 bp, Artificial Sequence
  tbs consensus sequence
  misc_feature (4)..(5): n is a, c, g, or t
  misc_feature (9)..(10): n is a, c, g, or t
  misc_feature (14)..(15): n is a, c, g, or t
  misc_feature (19)..(20): n is a, c, g, or t
  misc_feature (24)..(25): n is a, c, g, or t
  misc_feature (29)..(30): n is a, c, g, or t
  misc_feature (34)..(35): n is a, c, g, or t
  misc_feature (39)..(40): n is a, c, g, or t
  misc_feature (44)..(45): n is a, c, g, or t
  misc_feature (49)..(50): n is a, c, g, or t
  misc_feature (54)..(55): n is a, c, g, or t
kagnnkagnnkagnnkagnnkagnnkagnnkagnnkagnnkagnnkagnnkagnn

SEQ ID NO: 219 - DNA, 28 bp, Artificial Sequence
  wild type SL2-MSD-SL4(STD) sequence including SL2 loop region
ggcggcgactggtgagtacgccaaaaat

SEQ ID NO: 220 - DNA, 22 bp, Artificial Sequence
  wild type SL2-MSD-SL4(STD) sequence including SL4 loop region
gagatgggtgcgagagcgtcag

SEQ ID NO: 221 - DNA, 28 bp, Artificial Sequence
  MSD-1KO sequence including SL2 loop region
ggcggcgactgcagagtacgccaaaaat

SEQ ID NO: 222 - DNA, 22 bp, Artificial Sequence
  MSD-1KO sequence including SL4 loop region
gagatgggtgcgagagcgtcag

SEQ ID NO: 223 - DNA, 28 bp, Artificial Sequence
  MSD-2KO sequence including SL2 loop region
ggcggcgactgcagacaacgccaaaaat

SEQ ID NO: 224 - DNA, 22 bp, Artificial Sequence
  MSD-2KO sequence including SL4 loop region
gagatgggtgcgagagcgtcag

SEQ ID NO: 225 - DNA, 28 bp, Artificial Sequence
  MSD-3KO_1 sequence including SL2 loop region
ggcggcgactgcagacaacgccaaaaat

SEQ ID NO: 226 - DNA, 22 bp, Artificial Sequence
  MSD-3KO_1 sequence including SL4 loop region
gagatggctgcgagagcgtcag

SEQ ID NO: 227 - DNA, 28 bp, Artificial Sequence
  MSD-3KO_2 sequence including SL2 loop region
ggcggcgactgcagacaacgccaaaaat

SEQ ID NO: 228 - DNA, 22 bp, Artificial Sequence
  MSD-3KO_2 sequence including SL4 loop region
gagatgggcgcgagagcgtcag

SEQ ID NO: 229 - DNA, 28 bp, Artificial Sequence
  MSD-4KO_1 sequence including SL2 loop region
ggcggcgactgcagacaacgccaaaaat

SEQ ID NO: 230 - DNA, 22 bp, Artificial Sequence
  MSD-4KO_1 sequence including SL4 loop region
gagatggctgcgagagcatcag

SEQ ID NO: 231 - DNA, 28 bp, Artificial Sequence
  MSD-4KO_2 sequence including SL2 loop region
ggcggcgactgcagacaacgccaaaaat

SEQ ID NO: 232 - DNA, 22 bp, Artificial Sequence
  MSD-4KO_2 sequence including SL4 loop region
gagatgggcgcgagagcatcag

SEQ ID NO: 233 - DNA, 30 bp, Artificial Sequence
  MSD-2KOm5 sequence including SL2 loop region
ggaaggcaacagataaatatgccttaaaat

SEQ ID NO: 234 - DNA, 22 bp, Artificial Sequence
  MSD-2KOm5 sequence including SL4 loop region
gagatgggtgcgagagcgtcag

SEQ ID NO: 235 - DNA, 30 bp, Artificial Sequence; identical to SEQ ID NO: 233
  MSD-3KOm5_2 sequence including SL2 loop region

SEQ ID NO: 236 - DNA, 22 bp, Artificial Sequence
  MSD-3KOm5_2 sequence including SL4 loop region
gagatgggcgcgagagcgtcag

SEQ ID NO: 237 - DNA, 30 bp, Artificial Sequence; identical to SEQ ID NO: 233
  MSD-4KOm5_2 sequence including SL2 loop region

SEQ ID NO: 238 - DNA, 22 bp, Artificial Sequence
  MSD-4KOm5_2 sequence including SL4 loop region
gagatgggcgcgagagcatcag

SEQ ID NO: 239 - RNA, 11 nt, Artificial Sequence
  endogenous U1 snRNA
auacuuaccug

SEQ ID NO: 240 - RNA, 19 nt, Artificial Sequence
  wild type major splice donor site sequence, wt SL2-MSD(STD)
ggcgacuggugaguacgcc

SEQ ID NO: 241 - RNA, 19 nt, Artificial Sequence
  variant major splice donor site, MSD-2KO
ggcgacugcagacaacgcc

SEQ ID NO: 242 - RNA, 19 nt, Artificial Sequence
  variant major splice donor site, MSD-2KOm5
ggcaacagauaaauaugcc

(19) United States
(12) Patent Application Publication
(10) Pub. No.: US 2024/0052366 A1
(43) Pub. Date: Feb. 15, 2024

(54) Production of Lentiviral Vectors

(71) Applicant: Oxford BioMedica (UK) Limited, Oxford (GB)

(72) Inventors: Daniel Farley, Oxford (GB); Jordan Wright, Oxford (GB)

(21) Appl. No.: 17/799,679

(22) PCT Filed: Feb. 4, 2021
(86) PCT No.: PCT/GB2021/050247

§ 371 Date: Aug. 14, 2022

(30) Foreign Application Priority Data
Feb. 13, 2020 (GB) 2001996.4
Nov. 11, 2020 (GB) 2017820.8

(51) Int. Cl. C12N 15/86

(52) U.S. Cl. CPC C12N 15/86; C12N 2740/15043; C12N 2740/15052

(57) ABSTRACT

A nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated.

### CROSS-REFERENCE TO RELATED APPLICATIONS

This application is the national stage under 35 U.S.C. § 371 of International Application No. PCT/GB2021/050247, filed on Feb. 4, 2021, now expired, which claims the benefit of priority to United Kingdom Application No. 2001996.4, filed on Feb. 13, 2020, now expired, and to United Kingdom Application No. 2017820.8, filed on Nov. 11, 2020, now expired.

### FIELD OF THE INVENTION

The invention relates to the production of lentiviral vectors in eukaryotic cells. More specifically, the present invention relates to the inactivation of the major splice donor site and adjacent cryptic splice donor site in the vector genome. The present invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated. Methods and uses involving such a nucleotide sequence are also encompassed by the invention.

### BACKGROUND TO THE INVENTION

The development and manufacture of viral vectors towards vaccines and human gene therapy over the last several decades is well documented in scientific journals and in patents. The use of engineered viruses to deliver transgenes for therapeutic effect is wide-ranging. Contemporary gene therapy vectors based on RNA viruses such as γ-retroviruses and lentiviruses (Muhlebach, M. D. et al., 2010, Retroviruses: Molecular Biology, Genomics and Pathogenesis, 13:347-370; Antoniou, M. N., Skipper, K. A. & Anakok, O., 2013, Hum. Gene Ther., 24:363-374), and DNA viruses such as adenovirus (Capasso, C. et al., 2014, Viruses, 6:832-855) and adeno-associated virus (AAV) (Kotterman, M. A. & Schaffer, D. V., 2014, Nat. Rev. Genet., 15:445-451) have shown promise in a growing number of human disease indications. These include ex vivo modification of patient cells for hematological conditions (Morgan, R. A. & Kakarla, S., 2014, Cancer J., 20:145-150; Touzot, F. et al., 2014, Expert Opin. Biol. Ther., 14:789-798), and in vivo treatment of ophthalmic (Balaggan, K. S. & Ali, R. R., 2012, Gene Ther., 19:145-153), cardiovascular (Katz, M. G. et al., 2013, Hum. Gene Ther., 24:914-927), neurodegenerative diseases (Coune, P. G., Schneider, B. L. & Aebischer, P., 2012, Cold Spring Harb. Perspect. Med., 4:a009431) and tumor therapy (Pazarentzos, E. & Mazarakis, N. D., 2014, Adv. Exp. Med Biol., 818:255-280).

As the successes of these approaches in clinical trials begin to build towards regulatory approval and commercialisation, it is important to consider the safety aspects involved in administration of viral vectors to patients, for example in the context of vaccination and gene therapy.

There is an ongoing need in the art for viral vectors with improved safety profiles.

### SUMMARY OF THE INVENTION

The present invention is based on inactivation of both the major splice donor site and adjacent cryptic splice donor site in the packaging region of lentiviral vector genomes. The major splice donor site (MSD) present within lentiviral vector genomes is typically embedded within the packaging region containing highly structured RNA towards the 5′ region of the RNA.

Splice donor sites within retrovirus genomes have been shown to be important for viral RNA (vRNA) stability within the production cell. However, the present inventors show that the activity of the MSD within lentiviral vector genome expression cassettes can be highly promiscuous, and can very efficiently splice to strong or even weak cryptic splice acceptor sites within the internal expression cassette that are typically located >1350 bp downstream. Surprisingly, as much as 95% of the detectable cytoplasmic mRNA derived from the external promoter driving vRNA production is spliced depending on internal sequences.

For efficient vector production, it is the unspliced packageable vRNA that is the most desirable product, and this vector component is typically the limiting factor both in transient and stable transfection vector production settings. Moreover, if this aberrantly spliced mRNA encodes for the transgene of interest (for example splicing into the internal promoter-utr sequence), this mRNA will be exported and will be capable of being efficiently translated during vector production; this will occur independently of whether the internal promoter is a weak/silent (tissue specific) promoter.

In addition, the presence of the MSD in the vector backbone delivered in transduced (patient) cells has been shown by others to be utilised by the splicing machinery, when read-through transcription from upstream cellular promoters occurs (lentiviral vectors target active transcription sites), leading to potential aberrant splice-products with cellular exons. Therefore, there are several reasons why it is desirable to functionally mutate the MSD site from lentiviral vector genomes.

Others have generated early generation lentiviral vectors harbouring mutations within the MSD site but these vectors contained the inherent U3 promoter to drive transcription of the vRNA, and were therefore dependent on transactivation by tat, supplied in trans. Third generation lentiviral vectors replaced the U3 promoter with heterologous promoter elements, and do not require tat for transcription. The U3/tat-independence of 3^{rd} generation vectors is seen as an important advance in safety by regulators because tat is a transactivator of cellular genes and can play a role in oncogenesis.

In one aspect the present invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated.

It is demonstrated in the present Examples that inactivation of both the major splice donor site in the RNA genome of the lentiviral vector and the cryptic splice donor site 3′ to the major splice donor site provides an improvement over mutating either alone. As also demonstrated in the present Examples, the invention facilitates reduced transcriptional read-through into the integrated lentiviral vector in a target cell, for example by at least 2-fold.

In one aspect the nucleotide sequence according to the invention is for use in a U3 or tat-independent lentiviral vector system. In one aspect the lentiviral vector system may be a 3^{rd} generation lentiviral vector system as described herein.

The cryptic splice donor site is the first cryptic splice donor site or sequence 3′ to the major splice donor site. In one aspect the cryptic splice donor site or sequence is within 6 nucleotides of the major splice donor site. The major splice donor site and cryptic splice donor site may be mutated or deleted.

In one aspect the invention provides a nucleotide sequence encoding the RNA genome of the lentiviral vector, wherein the nucleotide sequence prior to inactivation of the splice sites comprises a sequence as set forth in any of SEQ ID NOs: 1, 3, 4, 9, 10 and/or 13. The nucleotide sequence may comprise a sequence with a mutation or deletion relative to the sequence as set forth in any of SEQ ID NOs: 1, 3, 4, 9, 10 and/or 13. In one aspect the sequence comprises SEQ ID NO:13.

In one aspect the nucleotide sequence comprises an inactivated major splice donor site which would otherwise have a cleavage site immediately upstream of nucleotide 1 of the major splice donor region (SEQ ID NO: 13).

In one aspect the nucleotide sequence comprises an inactivated major splice donor site and an inactivated cryptic splice donor site which would otherwise have a cleavage site immediately upstream of nucleotide 1, as well as between nucleotides 4 and 5 corresponding to nucleotides of the major splice donor region (SEQ ID NO: 13).

In one aspect the nucleotide sequence comprises an inactivated major splice donor site which would otherwise have a cleavage site between nucleotides corresponding to nucleotides 13 and 14 of SEQ ID NO:1.

In another aspect the nucleotide sequence of the major splice donor site prior to inactivation comprises the sequence as set forth in SEQ ID NO: 4. In one aspect the cryptic splice donor site prior to inactivation comprises the sequence as set forth in SEQ ID NO: 10.

The nucleotide sequence may comprise an inactivated cryptic splice donor site which would otherwise have a cleavage site between nucleotides corresponding to nucleotides 17 and 18 of SEQ ID NO:1.

In one aspect the nucleotide sequence according to the invention comprises a sequence as set forth in any of SEQ ID NOs: 2, 5, 6, 7, 8, 11, 12 and/or 14.

In a preferred aspect the nucleotide sequence comprises a sequence as set forth in SEQ ID NO: 14.

In a further aspect the nucleotide sequence does not comprise a sequence as set forth in SEQ ID NO:9.

Splicing activity from the major splice donor site and cryptic splice donor site of the RNA genome of the lentiviral vector may be suppressed or ablated, for example in transfected cells or in transduced cells.

In one aspect nucleotide sequence may be suitable for use in a lentiviral vector in a U3 or tat-independent system for vector production. As described herein, 3^{rd} generation lentiviral vectors are U3/tat-independent, and the nucleotide sequences according to the present invention may be used in the context of a 3^{rd} generation lentiviral vector. In one aspect of the invention tat is not provided in the lentiviral vector production system, for example tat is not provided in trans. In one aspect the cell or vector or vector production system as described herein does not comprise the tat protein. In one aspect of the invention HIV-1 U3 is not present in the lentiviral vector production system, for example HIV-1 U3 is not provided in cis to driven transcription of vector genome expression cassette.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence is for use in a tat-independent lentiviral vector.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence is produced in the absence of tat.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence has been transcribed independently of tat.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence is for use in a U3-independent lentiviral vector.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence has been transcribed independently of the U3 promoter.

In one aspect the invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated, wherein said nucleotide sequence has been transcribed by a heterologous promoter.

In one aspect, transcription of the nucleotide sequence as described herein is not dependent on the presence of U3. The nucleotide sequence may be derived from a U3-independent transcription event. The nucleotide sequence may be derived from a heterologous promoter. A nucleotide sequence as described herein may not comprise a native U3 promoter.

In one aspect, the nucleotide sequence as described herein may optionally further comprise a mutation in a cryptic splice donor site within the SL4 loop of the packaging sequence. In one aspect a GT dinucleotide in said cryptic splice donor site within the SL4 loop of the packaging sequence is mutated to GC.

In one aspect the nucleotide sequence further comprises a nucleotide of interest, which may give rise to a therapeutic effect.

In a further aspect the nucleotide sequence encoding the RNA genome of the lentiviral vector is a vector transgene expression cassette.

In another aspect of the invention the nucleotide sequence may further comprise a nucleotide sequence encoding a modified U1 snRNA, wherein said modified U1 snRNA has been modified to bind to a nucleotide sequence within the packaging region of an MSD-mutated lentiviral vector genome. The nucleotide sequence encoding the RNA genome of the lentiviral vector may be operably linked to the nucleotide sequence encoding the modified U1 snRNA. In one aspect the nucleotide sequence encoding a modified U1 snRNA is on a different nucleotide sequence, for example on a different plasmid, to the nucleotide sequence encoding the RNA genome of the lentiviral vector.

In another aspect the nucleotide sequence further comprises a tryptophan RNA-binding attenuation protein (TRAP) binding site, and also may comprise a Kozak sequence, wherein said TRAP binding site overlaps the Kozak sequence, or wherein said Kozak sequence comprises a portion of a TRAP binding site. The nucleotide sequence may also further comprise a multiple cloning site and a Kozak sequence, wherein said multiple cloning site is overlapping with or located downstream to the 3′ KAGN2-3 repeat of the TRAP binding site and upstream of the Kozak sequence. The nucleotide of interest may be operably linked to the TRAP binding site or the portion thereof.

In one aspect the present invention provides a nucleic acid sequence comprising a nucleotide of interest (transgene) and a tryptophan RNA-binding attenuation protein (TRAP) binding site, wherein the TRAP binding site overlaps with the transgene start codon ATG.

Any disclosures herein relating to a Kozak sequence/overlapping Kozak sequence are equally applicable (where appropriate) to equivalent aspects referring to the ATG of the start codon and overlap therewith.

In another aspect, the present invention provides a nucleic acid sequence comprising a nucleotide of interest and a Kozak sequence, wherein said Kozak sequence comprises a portion of a tryptophan RNA-binding attenuation protein (TRAP) binding site.

In one aspect the present invention provides a nucleic acid sequence comprising a nucleotide of interest (transgene) and a TRAP binding site, wherein the TRAP binding site comprises a portion of the transgene start codon ATG or vice versa.

The invention also provides an expression cassette comprising a nucleotide sequence encoding the RNA genome of the lentiviral vector as defined herein.

The invention also provides a viral vector production system comprising a set of nucleotide sequences, wherein the nucleotide sequences encode vector components including gag-pol, env, optionally rev, and the RNA genome of the lentiviral vector as defined herein.

In one aspect the invention also provides a cell comprising the nucleotide sequence encoding the RNA genome of the lentiviral vector, the expression cassette or the viral vector production system as defined herein.

A cell for producing lentiviral vectors may comprise:
a) nucleotide sequences encoding vector components including gag-pol and env, and optionally rev, and a nucleotide sequence encoding the RNA genome of the lentiviral vector as defined herein or the expression cassette as defined herein; or
   b) the viral vector production system as defined herein; and
(ii) optionally a nucleotide sequence encoding a modified U1 snRNA; and
(iii) optionally a nucleotide sequence encoding TRAP.

In the cell the splicing activity from the major splice donor site and/or splice donor region of the RNA genome of the lentiviral vector may be suppressed or ablated, for example during lentiviral vector production. In one aspect translation of the nucleotide of interest is repressed during lentiviral vector production.

The invention also provides a method for producing a lentiviral vector, comprising the steps of:
(i) Introducing:
   a) nucleotide sequences encoding vector components including gag-pol and env, and optionally rev, and the nucleotide sequence encoding the RNA genome of the lentiviral vector as defined herein or the expression cassette as defined herein; or
   b) the viral vector production system as defined herein; into a cell;
(ii) optionally selecting for a cell which comprises said nucleotide sequences encoding vector components and the RNA genome of the lentiviral vector;
(iii) culturing the cell under conditions suitable for the production of the lentiviral vector.

The method may additionally comprise introducing a nucleotide sequence encoding TRAP into the cell.

The method may additionally comprise introducing a nucleotide sequence encoding a modified U1 snRNA.

The invention also extends to a lentiviral vector produced by any of the methods as described herein.

In one aspect the invention provides use of the nucleotide sequence encoding the RNA genome of the lentiviral vector as defined herein, the expression cassette as defined herein, the viral vector production system as defined herein, or the cell as defined herein for producing a lentiviral vector, or for suppressing or ablating the splicing activity from the major splice donor site and/or splice donor region of the RNA genome of the lentiviral vector, for example in transfected cells or in transduced cells.

### DESCRIPTION OF THE FIGURES

FIG. 1: A schematic of a U1 snRNA molecule and an example of how to modify the targeting sequence for use in the invention. The endogenous non-coding RNA, U1 snRNA binds to the consensus splice donor site (5′-MAGGURR-3′) via the 5′-(AC)UUACCUG-3′ (grey highlighted) native splice donor targeting sequence during early steps of intron splicing. Stem loop I binds to U1A-70K protein that has been shown to be important for polyA suppression. Stem loop II binds to U1A protein, and the 5′-AUUUGUGG-3′ sequence binds to Sm proteins, which together with Stem loop IV, is important for U1 snRNA processing. In the invention, the modified U1 snRNA is modified to introduce a heterologous sequence that is complementary to a target sequence within the vector genome vRNA molecule at the site of the native splice donor targeting sequence; in this figure the example given directs the modified U1 snRNA to 15 nucleotides (256-270 relative to the first nucleotide of the vector genome molecule, 256U1) of a standard HIV-1 lentiviral vector genome (located in the SL1 loop if the packaging signal).

FIG. 2: Implications of aberrant splicing from the major splice donor site (MSD) within HIV-1 based lentiviral vectors. A. A schematic to show the typical configuration of a third generation (Self-inactivating (SIN)) lentiviral vector expression cassette, containing a functional major splice donor embedded within stem loop (SL2) of the packaging signal, and the types of mRNA generated during lentiviral vector production. The types of mRNA generated from a ‘standard’ lentiviral vector (LV) DNA cassette and a lentiviral vector DNA cassette with (a) functional mutation(s) in the MSD region (‘MSD-KO LV DNA cassette’) that suppress or ablate the promiscuous activity from the MSD are shown. For both cassettes, the full-length (Unspliced) vector RNA (vRNA) results from the co-expression of rev, which binds to the rev response element (RRE), and is generally believed to repress splicing from the MSD to splice acceptor 7 (sa7) included with RRE sequences. For a standard lentiviral vector DNA cassette, in the absence of rev, it is generally believed that splicing-out of all introns occurs efficiently (Spliced). However, ‘aberrant’ splice products can be made during lentiviral vector production wherein the MSD highly efficiently splices to splice acceptor sites or cryptic splice acceptor sites (“Aberrant’ spliced’), typically ‘over-looking’ the RRE-containing intron such that rev has minimal impact on this activity of the MSD. Lentiviral vector production can also be performed with co-expression of modified U1 snRNAs redirected to the packaging region of MSD-mutated lentiviral vector DNA cassettes. (Key: Pro, promoter; region from 5′R to gag contains the packaging element {ψ}; msd, major splice donor; cppt, central polypurine tract; Int, intron; sd/sa, splice donor/acceptor; GOI, gene of interest; grey arrow indicate position of forward {f} and reverse {r} primers to assess the proportion of Unspliced vRNA produced during 3^{rd} generation lentiviral vector production. Post-transcriptional regulatory element {PRE} not shown for clarity). B. Standard 3^{rd} generation lentiviral vector production was performed +/−rev in HEK293T cells and total RNA extracted from post-production cells. Total RNA was subjected to qPCR (SYBR green) using two primer sets (position marked in A): f+rT amplified total transcripts generated from the lentiviral vector expression cassette, and f+rUS amplified Unspliced transcripts; therefore the proportion of Unspliced-to-Total vRNA transcripts were calculated and plotted. The data indicates that the proportion of Unspliced vRNA relative to total during standard 3^{rd} generation lentiviral vector production is modest and varies according to the internal transgene cassette (in this case containing different promoters and the GFP gene); moreover, this proportion is only minimally increased by the action of rev.

FIG. 3: HIV-1 lentiviral vector genomes containing three different promoter-GFP expression cassettes (EF1a, EFS and CMV) were modified to functionally mutate the MSD resulting in the ‘MSD-2KO’ lentiviral vector genomes or back-bones (see FIG. 10A for description of mutations). Vectors were produced in HEK293T cells under standard protocol and titrated. The data shows that the functional mutation of the MSD (‘MSD-2KO’) results in up to 100-fold reduction in lentiviral vector titres.

FIG. 4: A A schematic to show the configuration of standard or MSD-mutated lentiviral vector expression cassettes encoding an EF1a-GFP internal expression cassette, and the types of mRNA generated during lentiviral vector production. (Key: Pro, promoter; region from 5′R to gag contains the packaging element {ψ}; msd, major splice donor; cppt, central polypurine tract; Int, intron; sd/sa, splice donor/acceptor; GOI, gene of interest; grey arrow indicate position of forward {f} and reverse {r} primers to assess the proportion of Unspliced vRNA produced during 3^{rd} generation lentiviral vector production. Post-transcriptional regulatory element {PRE} not shown for clarity). B i The standard lentiviral vectors or MSD-2KO lentiviral vectors were produced in HEK293T cells +/−tat, or 179U1, or 305U1, and titrated. ii Total cytoplasmic mRNA was extracted from post-production cells and analysed by RT-PCR/gel electrophoreses using primers (f+rG) that could detect the main ‘aberrant’ splice product from the SL2 splicing region SL2 splicing region to the EF1a splice acceptor. The data show that modified U1 snRNAs redirected to the 5′ packaging region of MSD-2KO lentiviral vector genome (vRNA) were able to increase titres of both standard and MSD-2KO lentiviral vectors in a manner similar to tat. The MSD-2KO mutation abolished detection of the ‘aberrant’ splice product, which is from the SL2 splicing region to the EF1a splice acceptor (see FIG. 4A). Importantly, the increase in titres by the modified U1 snRNAs was accompanied by maintenance of virtually undetectable ‘aberrant’ spliced product, in contrast to the use of tat.

FIG. 5: Standard lentiviral vectors or MSD-mutated lentiviral vectors encoding a GFP internal cassette driven by EF1a, EFS or CMV promoters were produced in HEK293T cells +/−256U1, and titrated. Enhanced lentiviral vector titres by the use of modified U1 snRNAs redirected to the 5′ packaging region are independent of promoter employed within the transgene cassette. The data shows that, in large part, the attenuating phenotype of the MSD-2KO mutation is rescued by the co-expression of modified U1 snRNA, and therefore this surprisingly boosts titre of MSD-mutated lentiviral vector genomes disproportionately compared to standard lentiviral vector genomes.

FIG. 6: Enhancement of MSD-mutated lentiviral vector titres by the use of modified U1 snRNAs redirected to the 5′ packaging region is not linked to suppression of potential activity of the 5′polyA signal within the 5′LTR. Previous reports show that mutation of the MSD can activate the polyA signal within the 5′R sequence of the 5′LTR of HIV-1 provirus and ‘mini-reporter’ cassettes, leading to premature termination of transcription; the binding of endogenous U1 snRNA and even redirected U1 snRNAs could block this polyA activity. A A GFP-polyA-GLuciferase reporter cassette was designed to assess the impact of two polyA signal mutants (pAM1=AAUAAA>AACAAA; pAKO=deletion of AAUAAA) and the wild type polyA signal (wt pA=AAUAAA) on transcriptional read-through the HIV-1 polyA site. Read-through the HIV-1 polyA signal was measurable by luciferase activity, which was normalized by GFP expression. B To test if modified U1 snRNAs were acting in a similar manner, a functional mutation (pAm1) in the 5′polyA signal was introduced into MSD-mutated lentiviral vector genomes harbouring EF1a-GFP or CMV-GFP expression cassettes. Standard and MSD-2KO lentiviral vector genomes harbouring EF1a-GFP or CMV-GFP expression cassettes were also used. Lentiviral vectors were produced in HEK293T cells +/−305U1, and titrated. The data indicate that functional ablation of the 5′polyA signal only led to a very modest increase in lentiviral vector titres, and therefore the observed increase in lentiviral vector titres afforded by the modified U1 snRNA—especially that of the MSD-2KO/polyA-mutated lentiviral vector genome—could not be attributed to suppression of 5′polyA activity.

FIG. 7: Several mutations were introduced into 305U1 and 256U1 modified U1 snRNAs, that are known to ablate U1-70K protein binding to SL1, U1A protein binding to SL2 or Sm protein binding to/near SL4 of the vector genome. Standard or MSD-mutated lentiviral vectors encoding an EF1a-GFP internal cassette were produced in the presence of these mutated modified U1 snRNAs and titrated, and titre values normalized to standard lentiviral vectors produced without modified U1 snRNAs. The data show that the enhancement in MSD-2KO lentiviral vector titres by modified U1 snRNAs is not dependent on U1-70K protein or U1A protein binding but is dependent on the Sm protein binding site. Thus, enhancement of MSD-2KO lentiviral vector titres by the use of modified U1 snRNAs redirected to the 5′ packaging region is not linked to any known function of U1 snRNA.

FIG. 8: Enhancement of MSD-mutated lentiviral vector titres by the use of modified U1 snRNAs containing targeting sequence of varying lengths. MSD-2KO lentiviral vectors containing the EF1a-GFP cassette were produced in HEK293T cells in the presence of modified U1 snRNAs targeting the ‘305’ region, wherein each modified U1 snRNA comprised a re-targeting sequence of different length in complementarity. The titre increase was observed when using modified U1 snRNAs containing complementarity lengths of 7-to-15 nucleotides, with maximal effect observed at 10 nucleotides or more.

FIG. 9: Maximal titre recovery/boost of an MSD-mutated lentiviral vector is observed when targeting modified U1 snRNAs to the packaging region of the vector genome RNA. An MSD-2KO lentiviral vector containing the EF1-GFP cassette was produced in the presence of modified U1 snRNAs with targeting sequence to sites along the length of the 5′end of the vector genome vRNA molecule comprising 15 nucleotide lengths of complementarity (or 9 nucleotides where indicated). Modified U1 snRNAs are named according to the first nucleotide of the targeting sequence site along the length of the 5′end of the vector genome vRNA molecule. The data bars for each modified U1 snRNA are aligned underneath the approximate labelled position of each known functional sequence within the 5′end of the vector genome vRNA (not to scale).

FIG. 10: A description of functional major splice donor mutations, their impact on lentiviral vector titres, and recovery by modified U1 snRNA. A The sequence of the stem loop 2 (SL2) region of ‘wild type’ HIV-1 (NL4-3; the ‘Standard’ sequence within current lentiviral vector genomes) is shown at the top. The sequence comprises the major splice donor site (MSD: consensus=CTGGT) and a cryptic splice donor site (that is utilized when the MSD site is mutated on its own (crSD: consensus=TGAGT). The nucleotides at the position of splicing when the splice donor site is used are identified in bold and by arrows. Four functional MSD mutations that ablate both the MSD and the crSD site splicing activities are described: MSD-2KO, which mutates the two ‘GT’ motifs from the MSD and the crSD sites (and is used widely in most Examples); MSD-2KOv2, which also comprises mutations that ablate both the MSD and crSD sites; MSD-2KOm5, which introduces an entirely new stem-loop structure lacking any splice donor sites; and ΔSL2, which deletes the SL2 sequence entirely. The substitutions introduced to the SL2 sequence in the MSD-2KO,MSD-2KOv2 and MSD-2KOm5 mutations are shown in lowercase italics. B The four lentiviral vector genome variants comprising functional MSD mutations (described in FIG. 10A) were cloned with EFS-GFP internal cassettes, and MSD-2KO or MSD-2KOm5 variants additionally cloned with EF1a-, CMV- or huPGK-GFP internal cassettes. Standard and MSD-mutated LVs were produced in HEK293T cells +1-256U1, and titrated. The data indicates that the degree of attenuation of lentiviral vector titre can vary according to the specific mutation, and that the MSD-2KOm5 variant generally produced a less attenuated phenotype. The modified U1 snRNA was capable of increasing lentiviral vector titres for the four lentiviral vector genome variants comprising functional MSD mutations when co-expressed during production. Titre increases were greatest when the 256U1 was expressed with MSD-mutated LV genomes harbouring the MSD-2KOm5 sequence.

FIG. 11: The modified U1 snRNA expression cassette can be located to the lentiviral vector genome plasmid backbone for ease of use in transient transfection protocols. Many of the Examples use a separate modified U1 snRNA expressing plasmid in co-transfection with lentiviral vector component plasmids in production of lentiviral vectors. To identify ‘permissive’ sites on the lentiviral vector genome plasmid backbone in order to be able to provide the modified U1 snRNA cassette in cis during transient transfection, three variants were cloned. A A schematic of the lentiviral vector genome variants providing the modified U1 snRNA cassette in cis during transient transfection. Version 1 (‘[cis] ver1’) and version 3 (‘[cis] ver3’) placed the modified U1 snRNA cassette between the resistance marker and the origin of replication such that the modified U1 snRNA cassette was inverted relative to the lentiviral vector genome cassette (the resistance marker orientation differed between ver1 and ver3), and version 2 (‘[cis] ver2’) placed the modified U1 snRNA cassette upstream and in the same orientation of the lentiviral vector genome cassette. (Key: Pro, promoter; region from 5′R to gag contains the packaging element {ψ}; msd, major splice donor {here shown as MSD-2KO}; RRE, rev response element; cppt, central polypurine tract; Transgenic, heterologous sequence comprising therapeutic payload; U1-Pro, U1 promoter; Term[3′box], U1 transcriptional terminator). B The three ‘cis’ versions of the MSD-2KO lentiviral vector genome plasmid containing an EF1a-GFP cassette were used to produce lentiviral vectors in HEK293T cells in parallel to the ‘trans’ approach, where the same MSD-2KO lentiviral vector genome (without inserted modified U1 snRNA cassette in the backbone) was produced +/−modified U1 snRNA supplied by co-transfection with a separate plasmid. The data shows that similarly to co-transfection of a separate modified U1 snRNA encoding plasmid, MSD-2KO lentiviral vector titre could be increased by use of the ‘cis’ lentiviral vector genomes.

FIG. 12: Aberrantly spliced mRNA expressing transgene during lentiviral vector production is abolished in MSD-2KO lentiviral vectors, reducing the amount of transgene mRNA required to be targeted by TRAP when utilising the TRiP system. A A schematic of a ‘TRiP’ lentiviral vector genome encoding an EF1a-GFP transgene cassette, wherein the TRAP binding site (tbs) is positioned within the 5′UTR of the cassette (supply of TRAP during vector production reduces transgene expression levels). During production of MSD-2KO lentiviral vectors, the full length, unspliced packagable vRNA and transgene mRNA are main forms of RNA produced from the lentiviral vector cassette (i) (when the transgene promoter is active during production). However, the promiscuous activity of the MSD in standard lentiviral vector genomes leads to additional ‘aberrant’ splice products that may encode the transgene (ii); this could occur independently of the internal transgene promoter i.e. a tissue specific promoter. (Key: Pro, promoter; region from 5′R to gag contains the packaging element {ψ}; msd, major splice donor; cppt, central polypurine tract; Int, intron; sd/sa, splice donor/acceptor; GOI, gene of interest; grey arrow indicate position of forward {f} and reverse {r} primers to assess the proportion of Unspliced vRNA produced during 3^{rd} generation lentiviral vector production. Post-transcriptional regulatory element {PRE} not shown for clarity). B Standard or MSD-2KO lentiviral vector genome plasmid containing an EF1a-GFP cassette were used to produce lentiviral vectors in HEK293T cells, and GFP expression scores generated (% GFP×MFI). Relative to the total amount of GFP produced in cultures during standard lentiviral vector production, the MSD-2KO had the substantial effect of reducing the amount of GFP produced even in the absence of TRAP. Accordingly, the repressive effects of TRAP were augmented by use of the MSD-2KO lentiviral vector genome, leading to much lower levels of GFP in cultures.

FIG. 13: Successful isolation of HEK293T cells stably expressing modified U1 snRNA that enables increase of standard or MSD-2KO lentiviral vectors demonstrates that the modified U1 snRNA cassettes can be introduced into lentiviral vector packaging and producer cell lines. Standard or MSD-2KO lentiviral vector genomes containing EFS-GFP cassettes were produced in HEK293T or HEK293T.305U1 (9nt variant) cells +/−additional 305U1 plasmid. The data indicate that stable cassettes expressing modified U1 snRNA can be introduced into cells without toxicity.

FIG. 14. Identification of optimal Kozak sequences that overlap with the 3′end of the tbs within transgene 5′UTR in order to position the tbs closer to the ATG initiation codon.

A. A schematic indicating the position and sequence of Kozak sequences in engineered variants conforming to the core consensus ‘RVVATG’ and the broader consensus ‘GNNRVVATG’ positioned in such a manner that the Kozak overlaps with the 3′end of the tbs so that KAGNN repeat(s) are maintained. This allows the tbs to be positioned closer to the ATG initiation codon to identify tbs-Kozak junction variants that enable improved transgene repression levels (+TRAP) by ‘hiding’ the ATG initiation codon within the TRAP-tbs complex. The maintenance of the consensus Kozak sequence enables transgene non-repressed levels (No TRAP) to be retained at high levels (i.e. modelling expression in vector-transduced cells).

B. The reporters were tested for non-repressed or repressed levels of GFP expression by co-transfection of the reporter plasmids with either pBlueScript (No TRAP) or pEF1α-TRAP (TRAP), respectively into HEK293T cells. Transfected cells were analysed by flow cytometry two days post-transfection, and GFP Expression scores (% GFP×median fluorescence intensity) generated and log-10 transformed. All the tbs-Kozak junction variant reporters maintained the same non-repressed GFP levels compared to the original configuration. Variants ‘0’, ‘2’ and ‘3’ displayed improved repressed levels compared to the original configuration (Standard deviation bars, n=3).

FIG. 15. Improvement in transgene repression in AAV vector genome plasmids by employing overlapping tbs-Kozak variants. Two ‘tbs-Kozak’ variants (0 and 3) were cloned into either EFS or huPGK promoter GFP reporter cassettes, additionally containing either the L33 or L12 Improved leader sequences. Non-overlapping tbs/Kozak variants were also cloned into EFS/huPGK-L33 cassettes; these differed only in the tbs-Kozak region (Original=[tbs]-ACAGCCACCATG; HpaI variant=[tbs-GAGTT]AACGCCACCATG). The reporters were tested for non-repressed or repressed levels of GFP expression by co-transfection of the reporter plasmids with either pBlueScript (No TRAP) or pEF1α-TRAP (TRAP), respectively. Transfected HEK293T cells (suspension, serum-free) were analysed by flow cytometry two days post-transfection, and GFP Expression scores (% GFP×median fluorescence intensity) generated and log-10 transformed. The data demonstrates that overlapping the tbs with the Kozak sequence allows improved repression of transgene expression by TRAP compared to the non-overlapping tbs/Kozak variants. (Standard deviation bars, n=3).

FIG. 16. Improvement in TRAP-mediated transgene repression in the context of the full length EF1a promoter.

A. Three ‘tbs-Kozak’ variants (0, 2 and 3) were cloned into an EF1a promoter GFP reporter cassette. After splicing the leader sequence comprises the L33 sequence (exon1) and a short 12 nt sequence from exon2 immediately upstream of the tbs.

B. The reporters were tested for non-repressed or repressed levels of GFP expression by co-transfection of the reporter plasmids with either pBlueScript (No TRAP) or pEF1α-TRAP (TRAP), respectively. Transfected HEK293T cells (suspension, serum-free) were analysed by flow cytometry two days post-transfection, and GFP Expression scores (% GFP×median fluorescence intensity) generated and log-10 transformed.

C. The GFP transgene cassettes were cloned into an HIV-1 lentiviral vector genome, and tested for non-repressed or repressed levels of GFP expression as described in B. The data demonstrates that overlapping the tbs with the Kozak sequence allows improved repression of transgene expression by TRAP. (Standard deviation bars, n=3).

FIG. 17. Testing TRAP-mediated transgene repression of overlapping tbs-Kozak variants in suspension (serum-free) HEK293T cells. The overlapping tbs-Kozak variants in Table IV were cloned into a pEF1α-GFP reporter plasmid and transfected into HEK293T cells +/−pTRAP, and flow cytometry performed 2 days post-transfection. A. GFP Expression scores (% GFP positive×MFI) were generated +/−TRAP and fold repression values generated and plotted. The variants are indicated along the x-axis, grouped according to the relative overlap of the 3′ tbs KAGNN repeat and the core Kozak sequence (‘overlap groups’—KAGatg, KAGNatg), KAGNNatg); the KAGNN is denoted by the black bracket and the core Kozak nucleotides by the grey line. Statistical analysis was performed comparing the following overlap groups (equal variance within overlap groups was confirmed by F-Test): fold-repression was statistically greater for KAGatg over KAGNatg (*p=0.0293); for KAGNatg over KAGNNatg (**p=0.00000482); and for KAGNNatg over the non-overlapping tbs (***p=0.000259), using two-tail T-Test. B. The non-repressed GFP Expression scores were plotted highest to lowest (left to right), and the two KAGatg overlap group variants tbskzkV0.G and tbskzkV0.T (showing greatest repression of all the variants in A) highlighted to show that the ‘G’ variant is preferred over the ‘T’ variant due to the former having the better ‘ON’ (non-repressed) levels.

FIG. 18. Improved repression of intron-containing promoters using an optimal overlapping tbs-Kozak variant.

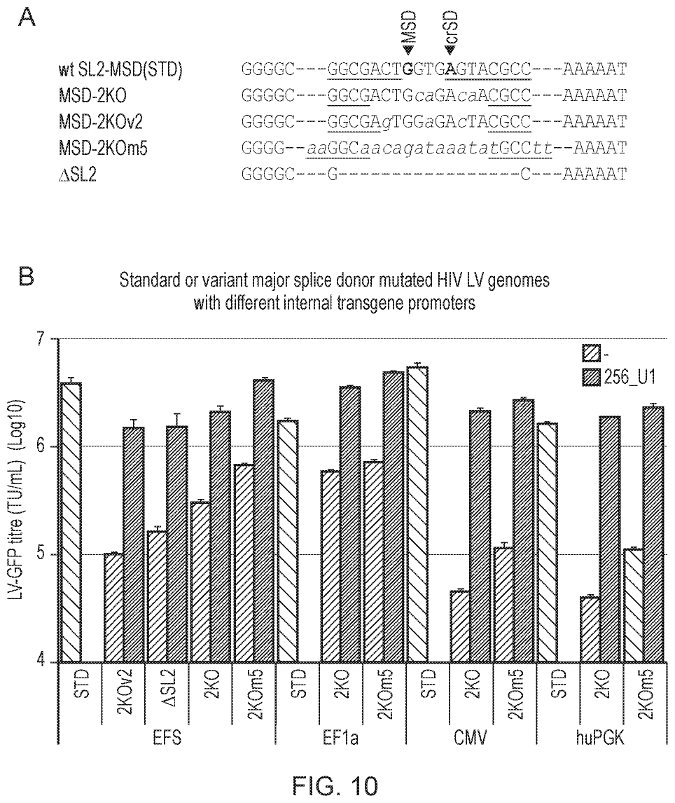

A. A schematic of expression cassettes used to exemplify the use of an overlapping tbs-Kozak variant compared to a non-overlapping tbs-Kozak variant. The widely used EF1a promoter sequence contains its own intron (see FIG. 10 and Example 5), as does the widely used CAG promoter. The CAG promoter is a very strong artificial promoter containing the CMV enhancer, the core promoter and exon1/intron sequence from the Chicken β-actin gene and the splice acceptor/exonic sequence from the Rabbit β-globin gene. In this work, the ‘EF1a-INT’ sequence from the EF1a promoter (containing exon 1 [L33]), all of the EF1a intron and splice acceptor, and 12 nucleotides from EF1a exon 2, was cloned into the CAG promoter, replacing the CAG exon/intron sequences. The ‘EF1a-INT’ sequence was also cloned into a CMV promoter construct. B. The constructs were evaluated for GFP expression and repression by TRAP in suspension (serum-free) HEK293T cells to model transgene expression during viral vector production. GFP Expression scores (% GFP×MFI) were generated and plotted, as well as fold-repression scores denoted in the presence of TRAP.

FIG. 19. Overview of improvements to 5′UTR sequences downstream of the tbs.

A. A schematic to show the DNA expression cassette of the 5′UTR encoding region of a TRAP-tbs repressible transgene cassette wherein a multicloning site (MCS) is inserted between the tbs and the initiation codon of the transgene (TRAP denoted by doughnut shape). The invention describes preferred, overlapping restriction enzymes sites that begin at/on the terminal KAGNN repeat of the tbs and contains up to five cloning sites upstream of the transgene initiation codon.

B. A schematic to show how the Kozak sequence of the transgene can be positioned such that it mostly or partially overlaps with the 3′ KAGNN repeat of the tbs; doing so can effectively ‘hide’ the main initiation codon within the TRAP-tbs complex making it even less accessible to translation machinery, leading to even lower ‘repressed’ levels of transgene expression.

C. A table to summarise preferred overlapping tbs and Kozak consensus sequences. The 3′ KAGNN repeat of the tbs is shown boxed and the core Kozak sequence is shown in bold.

FIG. 20: A. DNA sequences used to further exemplify mutation of splice donor sites in the HIV-1 packaging region of lentiviral vectors. The SL2 loop region (containing the MSD and crSD1) of the packaging sequence is boxed, as well as the SL4 loop (containing two further minor cryptic splice donor sites; crSD2 and crSD3). Key—Capitalised sequence is wild type (or aligns with it) as per ‘wt SL2-MSD-SL4 (STD)’, also denoting splice donor nucleotides in bold black. The ‘GT’ dinucleotide (considered critical for functional splice donor sites) is in bold grey where present. Dashes do not represent gaps in sequence but are used to space aligned sequences for greater clarity; forward slashes represent sequence not displayed between the SL2 and SL4 loops (and are all ‘missing’ sequence is from wild type HIV-1). Underlined sequence denotes nucleotides in the stem of SLs. Small case italicised sequence are modified sequences. 1/2/3/4K0′ denotes number of splice donors mutated in each variant. B. A schematic showing the predicted annealing of endogenous U1 snRNA to the wild type major splice donor site in standard lentiviral vector genomic RNA, as well as to the ‘MSD-2KO’ and ‘MSD-2KOm5’ variants. The ‘MSD-2KOm5’ variant is designed to anneal to endogenous U1 snRNA with greater stability (more complementarity) than ‘MSD-2KO’ (or indeed wild type), whilst still being functionally mutated in splice donor sites. In addition, ‘MSD-2KOm5’ contains flanking sequence to allow it to form a stem loop to minimise impact on packaging secondary structure (see A). C. Lentiviral vector genomes encoding the EF1a-GFP transgene cassette and a packaging region harbouring a standard (STD) MSD region (wild type sequence), or mutations either in the MSD only (MSD-1K0) or MSD/crSd1 (MSD-2KO) were constructed. LVs were produced in suspension (serum-free) HEK293T cells by transient transfection with or without 256U1 modified snRNA supplied in trans. PolyA-selected total mRNA from post-production cells was extracted and subjected to RT-PCR/agarose gel analysis, to identify global effects in aberrant splicing from the MSD region using a primer upstream of the MSD and downstream of the EF1a splice (See FIG. 23 for primer positions). The gel image indicates the position/type of aberrant splice products from the SL2/SL4 region to the EF1a splice acceptor (confirmed by sequencing of splice junctions [data not shown]). D. Lentiviral vectors titres of both MSD-1KO and MSD-2KO LVs were reduced compared to a standard LV but these were rescued by use of the modified U1 snRNA targeted to the packaging region of the mutated LVs (Y axis on log 10 scale).

FIG. 21: Further mutations to eliminate aberrant splicing from the crSD2 site in SL4 of the packaging sequence were added to LV genomes harbouring either MSD-2KO or MSD-2KOm5 mutations in SL2 (see FIG. 20). LVs were produced in suspension (serum-free) HEK293T cells by transient transfection with or without 256U1 modified snRNA supplied in trans. A. PolyA-selected total mRNA from post-production cells was extracted and subjected to RT-PCR/agarose gel analysis, to identify global effects in aberrant splicing from the MSD region in SL2 as well as from SL4, using a primer upstream of the MSD and downstream of the EF1a splice acceptor (See FIG. 23 for primer positions). The gel image indicates the position/type of aberrant splice products from the SL2/SL4 region to the EF1a splice acceptor (confirmed by sequencing of splice junctions [data not shown]). B. Lentiviral vectors titres of all MSD-1/2/3/4KO genomes were reduced compared to a standard LV but these were rescued by use of the modified U1 snRNA targeted to the packaging region of the mutated LVs (Y axis on log 10 scale).

FIG. 22: Integrated MSD-mutated LVs are subjected to lower rates of transcriptional read-through events compared to standard LVs. Lentiviral vectors insert semi-randomly into target cell DNA with a preference for transcriptionally active genes. This typically means that the integrated vector will site inside a cellular gene transcription unit. If the integrated LV resides downstream of a cellular promoter then some transcriptional read-through (‘read-in’) may occur into the LV unit. Current standard 3^{rd} generation LVs contain an intact MSD, and in theory, any read-in to the 5′LTR and packaging region may allow recruitment of endogenous U1 snRNA to the RNA. In such a situation, it is conceivable that the recruited U1 snRNA will suppress polyadenylation at the 5′ polyA site, leading to further elongation into the LV unit. Furthermore, splicing from the MSD may occur into downstream splice acceptors in the LV unit or even to cellular transcripts by trans-splicing. Use of MSD-mutated LVs are anticipated to be unable to recruit endogenous U1 snRNAs that lead to a splicing-competent precursor, and so may be less able to suppress polyadenylation at the 5′ polyA site. Thus, less transcriptional read-in is expected for this new type of vector. The positions of forward (f) and reverse primers (r) used for RT-qPCR of extracted total RNA from transduced cells to assess read-in transcription from upstream of the integrated LV cassettes are shown by grey arrows.

FIG. 23: Evidence that integrated MSD-mutated LVs are subjected to lower rates of transcriptional read-through events compared to standard LVs. The standard LV vector and MSD-mutant variant vectors generated in Example S2 were used to transduce either HEK293T cells or the primary cell 92BR at matched MOI. Only MSD-mutated vector preps produced in the presence of 256U1 were used as these gave comparable titres to the standard LV genome preps (see FIG. 21B). Transduced cells were passaged for 10 days to allow removal of non-integrated cDNA, and reduce signal of any vector RNA that may have been expressed from non-integrated vector cDNA. Host cell RNA was extracted and DNAse-treated prior to RT-qPCR analysis to detect read-in transcripts (see FIG. 22 for primer position). Detected HIV Psi RNA copies were normalised to detection of GAPDH signals (loading) and DNA copy-number of integrated vector, which was carried out separately. Detected HIV Psi DNA copies for standard LV made without 256U1 was set at 1, and all other data points set relative to this. Statistical comparison of all MSD-mutated variant vectors with standard vectors was carried out; t-Test (critical two-tail assuming equal variance [HEK293T] or unequal variance [92 BR]) after evaluation of mean variances by F-Test (critical one-tail) was carried out, revealing significant differences between the two groups (* p=0.00012 and ** p=0.000073).

### DETAILED DESCRIPTION OF THE INVENTION

RNA Splicing

As described herein, the present invention provides a nucleotide sequence encoding the RNA genome of a lentiviral vector, wherein the major splice donor site in the RNA genome of the lentiviral vector is inactivated, and wherein the cryptic splice donor site 3′ to the major splice donor site is inactivated.

RNA splicing is catalysed by a large RNA-protein complex called the spliceosome, which is comprised of five small nuclear ribonucleoproteins (snRNPs). The borders between introns and exons are marked by specific nucleotide sequences within a pre-mRNA, which delineate where splicing will occur. Such boundaries are referred to as “splice sites.” The term “splice site” refers to polynucleotides that are capable of being recognized by the splicing machinery of a eukaryotic cell as suitable for being cut and/or ligated to another splice site.

Splice sites allow for the excision of introns present in a pre-mRNA transcript. Typically, the 5′ splice boundary is referred to as the “splice donor site” or the “5′ splice site,” and the 3′ splice boundary is referred to as the “splice acceptor site” or the “3′ splice site.” Splice sites include, for example, naturally occurring splice sites, engineered or synthetic splice sites, canonical or consensus splice sites, and/or non-canonical splice sites, for example, cryptic splice sites.

Splice acceptor sites generally consist of three separate sequence elements: the branch point or branch site, a polypyrimidine tract and the acceptor consensus sequence. The branch point consensus sequence in eukaryotes is YNYTRAC (where Y is a pyrimidine, N is any nucleotide, and R is a purine). The 3′ acceptor splice site consensus sequence is YAG (where Y is a pyrimidine) (see, e.g., Griffiths et al., eds., Modern Genetic Analysis, 2nd edition, W.H. Freeman and Company, New York (2002)). The 3′ splice acceptor site typically is located at the 3′ end of an intron.

The terms “canonical splice site” or “consensus splice site” may be used interchangeably and refer to splice sites that are conserved across species.

Consensus sequences for the 5′ donor splice site and the 3′ acceptor splice site used in eukaryotic RNA splicing are well known in the art. These consensus sequences include nearly invariant dinucleotides at each end of the intron: GT at the 5′ end of the intron, and AG at the 3′ end of an intron.

The canonical splice donor site consensus sequence may be (for DNA) AG/GTRAGT (where A is adenosine, T is thymine, G is guanine, C is cytosine, R is a purine and “/” indicates the cleavage site). This conforms to the more general splice donor consensus sequence MAGGURR described herein. It is well known in the art that a splice donor may deviate from this consensus, especially in viral genomes where other constraints bear on the same sequence, such as secondary structure for example within a vRNA packaging region. Non-canonical splice sites are also well known in the art, albeit they occur rarely compared to the canonical splice donor consensus sequence.

By “major splice donor site” is meant the first (dominant) splice donor site in the viral vector genome, encoded and embedded within the native viral RNA packaging sequence typically located in the 5′ region of the viral vector nucleotide sequence.

In one aspect the nucleotide sequence does not contain an active major splice donor site, that is splicing does not occur from the major splice donor site in said nucleotide sequence, and splicing activity from the major splice donor site is ablated.

The major splice donor site is located in the 5′ packaging region of a lentiviral genome.

In the case of the HIV-1 virus, the major splice donor consensus sequence is (for DNA) TG/GTRAGT (where A is adenosine, T is thymine, G is guanine, C is cytosine, R is a purine and “/” indicates the cleavage site).

In one aspect of the invention, the splice donor region, i.e. the region of the vector genome which comprises the major splice donor site prior to mutation may have the following sequence:

(SEQ ID NO: 1)
GGGGCGGCGACTGGTGAGTACGCCAAAAAT

In one aspect of the invention the mutated splice donor region may comprise the sequence:

(SEQ ID NO: 2-MSD-2KO)
GGGGCGGCGACTGCAGACAACGCCAAAAAT

In one aspect of the invention the mutated splice donor region may comprise the sequence:

(SEQ ID NO: 11-MSD-2KOv2)
GGGGCGGCGAGTGGAGACTACGCCAAAAAT

In one aspect of the invention the mutated splice donor region may comprise the sequence:

(SEQ ID NO: 12-MSD-2KOm5)
GGGGAAGGCAACAGATAAATATGCCTTAAAAT

In one aspect of the invention prior to modification the splice donor region may comprise the sequence:

(SEQ ID NO: 9)
GGCGACTGGTGAGTACGCC

This sequence is also referred to herein as the “stem loop 2” region (SL2). This sequence may form a stem loop structure in the splice donor region of the vector genome. In one aspect of the invention this sequence (SL2) may have been deleted from the nucleotide sequence according to the invention as described herein.

As such, the invention encompasses a nucleotide sequence that does not comprise SL2. The invention encompasses a nucleotide sequence that does not comprise a sequence according to SEQ ID NO:9.

In one aspect of the invention the major splice donor site may have the following consensus sequence, wherein R is a purine and “/” is the cleavage site:

(SEQ ID NO: 3)
TG/GTRAGT

In one aspect, R may be guanine (G).

In one aspect of the invention, the major splice donor and cryptic splice donor region may have the following core sequence, wherein “/” are the cleavage sites at the major splice donor and cryptic splice donor sites:

(SEQ ID NO: 13)
/GTGA/GTA.

In one aspect of the invention the MSD-mutated vector genome may have at least two mutations in the major splice donor and cryptic splice donor ‘region’ (SEQ ID NO:13), wherein the first and second ‘GT’ nucleotides are the immediately 3′ of the major splice donor and cryptic splice donor nucleotides respectively

In one aspect of the invention the major splice donor consensus sequence is CTGGT (SEQ ID NO:4). The major splice donor site may contain the sequence CTGGT.

In one aspect the nucleotide sequence, prior to inactivation of the splice sites, comprises the sequence as set forth in any of SEQ ID NOs: 1, 3, 4, 9, 10 and/or 13.

In one aspect the nucleotide sequence comprises an inactivated major splice donor site which would otherwise have a cleavage site between nucleotides corresponding to nucleotides 13 and 14 of SEQ ID NO:1.

According to the invention as described herein, the nucleotide sequence also contains an inactive cryptic splice donor site. In one aspect the nucleotide sequence does not contain an active cryptic splice donor site adjacent to (3′ of) the major splice donor site, that is to say that splicing does not occur from the adjacent cryptic splice donor site, and splicing from the cryptic splice donor site is ablated.

The term “cryptic splice donor site” refers to a nucleic acid sequence which does not normally function as a splice donor site or is utilised less efficiently as a splice donor site due to the adjacent sequence context (e.g. the presence of a nearby ‘preferred’ splice donor), but can be activated to become a more efficient functioning splice donor site by mutation of the adjacent sequence (e.g. mutation of the nearby ‘preferred’ splice donor).

In one aspect the cryptic splice donor site is the first cryptic splice donor site 3′ of the major splice donor.

In one aspect the cryptic splice donor site is within 6 nucleotides of the major splice donor site on the 3′ side of the major splice donor site. Preferably the cryptic splice donor site is within 4 or 5, preferably 4, nucleotides of the major splice donor cleavage site.

In one aspect of the invention the cryptic splice donor site has the consensus sequence TGAGT (SEQ ID NO:10).

In one aspect the nucleotide sequence comprises an inactivated cryptic splice donor site which would otherwise have a cleavage site between nucleotides corresponding to nucleotides 17 and 18 of SEQ ID NO:1.

In one aspect of the invention the major splice donor site and/or adjacent cryptic splice donor site contain a “GT” motif. In one aspect of the invention both the major splice donor site and adjacent cryptic splice donor site contain a “GT” motif which is mutated. The mutated GT motifs may inactivate splice activity from both the major splice donor site and adjacent cryptic splice donor site. An example of such a mutation is referred to herein as “MSD-2KO”.

In one aspect the splice donor region may comprise the following sequence:

(SEQ ID NO: 5)
CAGACA

For example, in one aspect the mutated splice donor region may comprise the following sequence:

(SEQ ID NO: 6)
GGCGACTGCAGACAACGCC

A further example of an inactivating mutation is referred to herein as “MSD-2KOv2”.

In one aspect the mutated splice donor region may comprise the following sequence:

(SEQ ID NO: 7)
GTGGAGACT

For example, in one aspect the mutated splice donor region may comprise the following sequence:

(SEQ ID NO: 8)
GGCGAGTGGAGACTACGCC

For example, in one aspect the mutated splice donor region may comprise the following sequence:

(SEQ ID NO: 14)
AAGGCAACAGATAAATATGCCTT

In one aspect the stem loop 2 region as described above may be deleted from the splice donor region, resulting in inactivation of both the major splice donor site and the adjacent cryptic splice donor site. Such a deletion is referred to herein as “ΔSL2”.

A variety of different types of mutations can be introduced into the nucleic acid sequence in order to inactivate the major and adjacent cryptic splice donor sites.

In one aspect the mutation is a functional mutation to ablate or suppress splicing activity in the splice region. The nucleotide sequence as described herein may contain a mutation or deletion in any of the nucleotides in any of SEQ ID NOs: 1, 3, 4, 9, 10 and/or 13. Suitable mutations will be known to one skilled in the art, and are described herein.

For example, a point mutation can be introduced into the nucleic acid sequence. The term “point mutation,” as used herein, refers to any change to a single nucleotide. Point mutations include, for example, deletions, transitions, and transversions; these can be classified as nonsense mutations, missense mutations, or silent mutations when present within protein coding sequence. A “nonsense” mutation produces a stop codon. A “missense” mutation produces a codon that encodes a different amino acid. A “silent” mutation produces a codon that encodes either the same amino acid or a different amino acid that does not alter the function of the protein. One or more point mutations can be introduced into the nucleic acid sequence comprising the cryptic splice donor site. For example, the nucleic acid sequence comprising the cryptic splice site can be mutated by introducing two or more point mutations therein.

At least two point mutations can be introduced in several locations within the nucleic acid sequence comprising the major splice donor and cryptic splice donor sites to achieve attenuation of splicing from the splice donor region. In one aspect the mutations may be within the four nucleotides at the splice donor cleavage site; in the canonical splice donor consensus sequence this is A¹G²/G³T⁴, wherein “/” is the cleavage site. It is well known in the art that a splice donor cleavage site may deviate from this consensus, especially in viral genomes where other constraints bear on the same sequence, such as secondary structure for example within a vRNA packaging region. It is well known that the G³T⁴ dinucleotide is generally the least variable sequence within the canonical splice donor consensus sequence, and mutations to the G³ and or T⁴ will most likely achieve the greatest attenuating effect. For example, for the major splice donor site in HIV-1 viral vector genomes this can be T¹G²/G³T⁴, wherein “/” is the cleavage site. For example, for the cryptic splice donor site in HIV-1 viral vector genomes this can be G¹A²/G³T⁴, wherein “/” is the cleavage site. Additionally, the point mutation(s) can be introduced adjacent to a splice donor site. For example, the point mutation can be introduced upstream or downstream of a splice donor site. In embodiments where the nucleic acid sequence comprising a major and/or cryptic splice donor site is mutated by introducing multiple point mutations therein, the point mutations can be introduced upstream and/or downstream of the cryptic splice donor site.

As described herein, and as shown in the Examples, the nucleotide sequence encoding the RNA genome of the lentiviral vector according to the invention may optionally further comprise a mutation in a cryptic splice donor site within the SL4 loop of the packaging sequence. In one aspect a GT dinucleotide in said cryptic splice donor site within the SL4 loop of the packaging sequence is mutated to GC.

Construction of Splice Site Mutants

Splice site mutants of the present invention may be constructed using a variety of techniques. For example, mutations may be introduced at particular loci by synthesising oligonucleotides containing a mutant sequence, flanked by restriction sites enabling ligation to fragments of the native sequence. Following ligation, the resulting reconstructed sequence comprises a derivative having the desired nucleotide insertion, substitution, or deletion.

Other known techniques allowing alterations of DNA sequence include recombination approaches such as Gibson assembly, Golden-gate cloning and In-fusion.

Alternatively, oligonucleotide-directed site-specific (or segment specific) mutagenesis procedures may be employed to provide an altered sequence according to the substitution, deletion, or insertion required. Deletion or truncation derivatives of splice site mutants may also be constructed by utilising convenient restriction endonuclease sites adjacent to the desired deletion.

Subsequent to restriction, overhangs may be filled in, and the DNA religated.

Exemplary methods of making the alterations set forth above are disclosed by Sambrook et al. (Molecular cloning: A Laboratory Manual, 2d Ed., Cold Spring Harbor Laboratory Press, 1989).

Splice site mutants may also be constructed utilising techniques of PCR mutagenesis, chemical mutagenesis, chemical mutagenesis (Drinkwater and Klinedinst, 1986) by forced nucleotide misincorporation (e.g., Liao and Wise, 1990), or by use of randomly mutagenised oligonucleotides (Horwitz et al., 1989).

The present invention also provides a method for producing a lentiviral vector nucleotide sequence, comprising the steps of:
(i) providing a nucleotide sequence encoding the RNA genome of a lentiviral vector as described herein; and
(ii) mutating the major splice donor site and cryptic splice donor site as described herein in said nucleotide sequence.

Vector/Expression Cassette

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into and expressed by a target cell. The vector may facilitate the integration of the nucleotide sequence encoding a viral vector component to maintain the nucleotide sequence encoding the viral vector component and its expression within the target cell.

The vector may be or may include an expression cassette (also termed an expression construct). Expression cassettes as described herein comprise regions of nucleic acid containing sequences capable of being transcribed. Thus, sequences encoding mRNA, tRNA and rRNA are included within this definition.

The vector may contain one or more selectable marker genes (e.g. a neomycin resistance gene) and/or traceable marker gene(s) (e.g. a gene encoding green fluorescent protein (GFP)). Vectors may be used, for example, to infect and/or transduce a target cell. The vector may further comprise a nucleotide sequence enabling the vector to replicate in the host cell in question, such as a conditionally replicating oncolytic vector.

The term “cassette”—which is synonymous with terms such as “conjugate”, “construct” and “hybrid”—includes a polynucleotide sequence directly or indirectly attached to a promoter. The expression cassettes for use in the invention comprise a promoter for the expression of the nucleotide sequence encoding a viral vector component and optionally a regulator of the nucleotide sequence encoding the viral vector component. Preferably the cassette comprises at least a polynucleotide sequence operably linked to a promoter.

The choice of expression cassette, e.g. plasmid, cosmid, virus or phage vector, will often depend on the host cell into which it is to be introduced. The expression cassette can be a DNA plasmid (supercoiled, nicked or linearised), minicircle DNA (linear or supercoiled), plasmid DNA containing just the regions of interest by removal of the plasmid backbone by restriction enzyme digestion and purification, DNA generated using an enzymatic DNA amplification platform e.g. doggybone DNA (dbDNA™) where the final DNA used is in a closed ligated form or where it has been prepared (e.g. restriction enzyme digestion) to have open cut ends.

Lentiviral Vector Production Systems and Cells

A lentiviral vector production system comprises a set of nucleotide sequences encoding the components required for production of the lentiviral vector. Accordingly, a vector production system comprises a set of nucleotide sequences which encode the viral vector components necessary to generate lentiviral vector particles.

“Viral vector production system” or “vector production system” or “production system” is to be understood as a system comprising the necessary components for lentiviral vector production.

In one embodiment, the viral vector production system comprises nucleotide sequences encoding Gag and Gag/Pol proteins, and Env protein and the vector genome sequence. The production system may optionally comprise a nucleotide sequence encoding the Rev protein, or functional substitute thereof.

In one embodiment, the viral vector production system comprises modular nucleic acid constructs (modular constructs). A modular construct is a DNA expression construct comprising two or more nucleic acids used in the production of lentiviral vectors. A modular construct can be a DNA plasmid comprising two or more nucleic acids used in the production of lentiviral vectors. The plasmid may be a bacterial plasmid. The nucleic acids can encode for example, gag-pol, rev, env, vector genome. In addition, modular constructs designed for generation of packaging and producer cell lines may additionally need to encode transcriptional regulatory proteins (e.g. TetR, CymR) and/or translational repression proteins (e.g. TRAP) and selectable markers (e.g. Zeocin™, hygromycin, blasticidin, puromycin, neomycin resistance genes). Suitable modular constructs for use in the present invention are described in EP 3502260, which is hereby incorporated by reference in its entirety.

As the modular constructs for use in accordance with the present invention contain nucleic acid sequences encoding two or more of the retroviral components on one construct, the safety profile of these modular constructs has been considered and additional safety features directly engineered into the constructs. These features include the use of insulators for multiple open reading frames of retroviral vector components and/or the specific orientation and arrangement of the retroviral genes in the modular constructs. It is believed that by using these features the direct read-through to generate replication-competent viral particles will be prevented.

The nucleic acid sequences encoding the viral vector components may be in reverse and/or alternating transcriptional orientations in the modular construct. Thus, the nucleic acid sequences encoding the viral vector components are not presented in the same 5′ to 3′ orientation, such that the viral vector components cannot be produced from the same mRNA molecule. The reverse orientation may mean that at least two coding sequences for different vector components are presented in the ‘head-to-head’ and ‘tail-to-tail’ transcriptional orientations. This may be achieved by providing the coding sequence for one vector component, e.g. env, on one strand and the coding sequence for another vector component, e.g. rev, on the opposing strand of the modular construct. Preferably, when coding sequences for more than two vector components are present in the modular construct, at least two of the coding sequences are present in the reverse transcriptional orientation. Accordingly, when coding sequences for more than two vector components are present in the modular construct, each component may be orientated such that it is present in the opposite 5′ to 3′ orientation to all of the adjacent coding sequence(s) for other vector components to which it is adjacent, i.e. alternating 5′ to 3′ (or transcriptional) orientations for each coding sequence may be employed.

The modular construct for use according to the present invention may comprise nucleic acid sequences encoding two or more of the following vector components: gag-pol, rev, env, vector genome. The modular construct may comprise nucleic acid sequences encoding any combination of the vector components. In one embodiment, the modular construct may comprise nucleic acid sequences encoding:
i) the RNA genome of the retroviral vector and rev, or a functional substitute thereof;
ii) the RNA genome of the retroviral vector and gag-pol;
iii) the RNA genome of the retroviral vector and env;
iv) gag-pol and rev, or a functional substitute thereof;
v) gag-pol and env;
vi) env and rev, or a functional substitute thereof;
vii) the RNA genome of the retroviral vector, rev, or a functional substitute thereof, and gag-pol;
viii) the RNA genome of the retroviral vector, rev, or a functional substitute thereof, and env;
ix) the RNA genome of the retroviral vector, gag-pol and env; or
x) gag-pol, rev, or a functional substitute thereof, and env,
   wherein the nucleic acid sequences are in reverse and/or alternating orientations.

In one embodiment, a cell for producing retroviral vectors may comprise nucleic acid sequences encoding any one of the combinations i) to x) above, wherein the nucleic acid sequences are located at the same genetic locus and are in reverse and/or alternating orientations. The same genetic locus may refer to a single extrachromosomal locus in the cell, e.g. a single plasmid, or a single locus (i.e. a single insertion site) in the genome of the cell. The cell may be a stable or transient cell for producing retroviral vectors, e.g. lentiviral vectors. In one aspect the cell does not comprise tat.

The DNA expression construct can be a DNA plasmid (supercoiled, nicked or linearised), minicircle DNA (linear or supercoiled), plasmid DNA containing just the regions of interest by removal of the plasmid backbone by restriction enzyme digestion and purification, DNA generated using an enzymatic DNA amplification platform e.g. doggybone DNA (dbDNA™) where the final DNA used is in a closed ligated form or where it has been prepared (e.g. restriction enzyme digestion) to have open cut ends.

In one embodiment, the lentiviral vector is derived from HIV-1, HIV-2, SIV, FIV, BIV, EIAV, CAEV or Visna lentivirus.

A “viral vector production cell”, “vector production cell”, or “production cell” is to be understood as a cell that is capable of producing a lentiviral vector or lentiviral vector particle. Lentiviral vector production cells may be “producer cells” or “packaging cells”. One or more DNA constructs of the viral vector system may be either stably integrated or episomally maintained within the viral vector production cell. Alternatively, all the DNA components of the viral vector system may be transiently transfected into the viral vector production cell. In yet another alternative, a production cell stably expressing some of the components may be transiently transfected with the remaining components required for vector production.

As used herein, the term “packaging cell” refers to a cell which contains the elements necessary for production of lentiviral vector particles but which lacks the vector genome. Optionally, such packaging cells contain one or more expression cassettes which are capable of expressing viral structural proteins (such as gag, gag/pol and env).

Producer cells/packaging cells can be of any suitable cell type. Producer cells are generally mammalian cells but can be, for example, insect cells.

As used herein, the term “producer/production cell” or “vector producing/production cell” refers to a cell which contains all the elements necessary for production of lentiviral vector particles. The producer cell may be either a stable producer cell line or derived transiently or may be a stable packaging cell wherein the retroviral genome is transiently expressed.

In the methods of the invention, the vector components may include gag, env, rev and/or the RNA genome of the lentiviral vector when the viral vector is a lentiviral vector. The nucleotide sequences encoding vector components may be introduced into the cell either simultaneously or sequentially in any order.

The vector production cells may be cells cultured in vitro such as a tissue culture cell line. In some embodiments of the methods and uses of the invention, suitable production cells or cells for producing a lentiviral vector are those cells which are capable of producing viral vectors or viral vector particles when cultured under appropriate conditions. Thus, the cells typically comprise nucleotide sequences encoding vector components, which may include gag, env, rev and the RNA genome of the lentiviral vector. Suitable cell lines include, but are not limited to, mammalian cells such as murine fibroblast derived cell lines or human cell lines. They are generally mammalian, including human cells, for example HEK293T, HEK293, CAP, CAP-T or CHO cells, but can be, for example, insect cells such as SF9 cells. Preferably, the vector production cells are derived from a human cell line. Accordingly, such suitable production cells may be employed in any of the methods or uses of the present invention.

Methods for introducing nucleotide sequences into cells are well known in the art and have been described previously. Thus, the introduction into a cell of nucleotide sequences encoding vector components including gag, env, rev and the RNA genome of the lentiviral vector, using conventional techniques in molecular and cell biology is within the capabilities of a person skilled in the art.

Stable production cells may be packaging or producer cells. To generate producer cells from packaging cells the vector genome DNA construct may be introduced stably or transiently. Packaging/producer cells can be generated by transducing a suitable cell line with a retroviral vector which expresses one of the components of the vector, i.e. a genome, the gag-pol components and an envelope as described in WO 2004/022761.

Alternatively, the nucleotide sequence can be transfected into cells and then integration into the production cell genome occurs infrequently and randomly. The transfection methods may be performed using methods well known in the art. For example, a stable transfection process may employ constructs which have been engineered to aid concatemerisation. In another example, the transfection process may be performed using calcium phosphate or commercially available formulations such as Lipofectamine™ 2000CD (Invitrogen, CA), FuGENE® HD or polyethylenimine (PEI). Alternatively nucleotide sequences may be introduced into the production cell via electroporation. The skilled person will be aware of methods to encourage integration of the nucleotide sequences into production cells. For example, linearising a nucleic acid construct can help if it is naturally circular. Less random integration methodologies may involve the nucleic acid construct comprising of areas of shared homology with the endogenous chromosomes of the mammalian host cell to guide integration to a selected site within the endogenous genome. Furthermore, if recombination sites are present on the construct then these can be used for targeted recombination. For example, the nucleic acid construct may contain a loxP site which allows for targeted integration when combined with Cre recombinase (i.e. using the Cre/lox system derived from P1 bacteriophage). Alternatively or additionally, the recombination site is an att site (e.g. from λ phage), wherein the att site permits site-directed integration in the presence of a lambda integrase. This would allow the lentiviral genes to be targeted to a locus within the host cellular genome which allows for high and/or stable expression.

Other methods of targeted integration are well known in the art. For example, methods of inducing targeted cleavage of genomic DNA can be used to encourage targeted recombination at a selected chromosomal locus. These methods often involve the use of methods or systems to induce a double strand break (DSB) e.g. a nick in the endogenous genome to induce repair of the break by physiological mechanisms such as non-homologous end joining (NHEJ). Cleavage can occur through the use of specific nucleases such as engineered zinc finger nucleases (ZFN), transcription-activator like effector nucleases (TALENs), using CRISPR/Cas9 systems with an engineered crRNA/tracr RNA (‘single guide RNA’) to guide specific cleavage, and/or using nucleases based on the Argonaute system (e.g., from T. thermophilus).

Packaging/producer cell lines can be generated by integration of nucleotide sequences using methods of just lentiviral transduction or just nucleic acid transfection, or a combination of both can be used.

Methods for generating retroviral vectors from production cells and in particular the processing of retroviral vectors are described in WO 2009/153563.

In one embodiment, the production cell may comprise the RNA-binding protein (e.g. tryptophan RNA-binding attenuation protein, TRAP) and/or the Tet Repressor (TetR) protein or alternative regulatory proteins (e.g. CymR).

Production of lentiviral vector from production cells can be via transfection methods, from production from stable cell lines which can include induction steps (e.g. doxycycline induction) or via a combination of both. The transfection methods may be performed using methods well known in the art, and examples have been described previously.

Production cells, either packaging or producer cell lines or those transiently transfected with the lentiviral vector encoding components are cultured to increase cell and virus numbers and/or virus titres. Culturing a cell is performed to enable it to metabolize, and/or grow and/or divide and/or produce viral vectors of interest according to the invention. This can be accomplished by methods well known to persons skilled in the art, and includes but is not limited to providing nutrients for the cell, for instance in the appropriate culture media. The methods may comprise growth adhering to surfaces, growth in suspension, or combinations thereof. Culturing can be done for instance in tissue culture flasks, tissue culture multiwell plates, dishes, roller bottles, wave bags or in bioreactors, using batch, fed-batch, continuous systems and the like. In order to achieve large scale production of viral vector through cell culture it is preferred in the art to have cells capable of growing in suspension. Suitable conditions for culturing cells are known (see e.g. Tissue Culture, Academic Press, Kruse and Paterson, editors (1973), and R.I. Freshney, Culture of animal cells: A manual of basic technique, fourth edition (Wiley-Liss Inc., 2000, ISBN 0-471-34889-9).

Preferably cells are initially ‘bulked up’ in tissue culture flasks or bioreactors and subsequently grown in multi-layered culture vessels or large bioreactors (greater than 50 L) to generate the vector producing cells for use in the present invention.

Preferably cells are grown in a suspension mode to generate the vector producing cells for use in the present invention.

Lentiviral Vectors

Lentiviruses are part of a larger group of retroviruses. A detailed list of lentiviruses may be found in Coffin et al (1997) “Retroviruses” Cold Spring Harbour Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of human auto-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), feline immunodeficiency virus (FIV), Maedi visna virus (MVV) and bovine immunodeficiency virus (BIV). In one embodiment, the lentiviral vector is derived from HIV-1, HIV-2, SIV, FIV, BIV, EIAV, CAEV or Visna lentivirus.

The lentivirus family differs from retroviruses in that lentiviruses have the capability to infect both dividing and non-dividing cells (Lewis et al (1992) EMBO J 11(8):3053-3058 and Lewis and Emerman (1994) J Virol 68 (1):510-516). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects or transduces target cells and expresses NOI.

The lentiviral vector may be used to replicate the NOI in a compatible target cell in vitro. Thus, described herein is a method of making proteins in vitro by introducing a vector of the invention into a compatible target cell in vitro and growing the target cell under conditions which result in expression of the NOI. Protein and NOI may be recovered from the target cell by methods well known in the art. Suitable target cells include mammalian cell lines and other eukaryotic cell lines.

In some aspects the vectors may have “insulators”—genetic sequences that block the interaction between promoters and enhancers, and act as a barrier reducing read-through from an adjacent gene.

In one embodiment the insulator is present between one or more of the lentiviral nucleic acid sequences to prevent promoter interference and read-thorough from adjacent genes. If the insulators are present in the vector between one or more of the lentiviral nucleic acid sequences, then each of these insulated genes may be arranged as individual expression units.

The basic structure of retroviral and lentiviral genomes share many common features such as a 5′ LTR and a 3′ LTR, between or within which are located a packaging signal to enable the genome to be packaged, a primer binding site, integration sites to enable integration into a target cell genome and gag/pol and env genes encoding the packaging components—these are polypeptides required for the assembly of viral particles. Lentiviruses have additional features, such as the rev gene and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a typical lentiviral vector as described herein, at least part of one or more protein coding regions essential for replication may be removed from the virus; for example, gag/pol and env may be absent or not functional. This makes the viral vector replication-defective.

The lentiviral vector may be derived from either a primate lentivirus (e.g. HIV-1) or a non-primate lentivirus (e.g. EIAV).

In general terms, a typical retroviral vector production system involves the separation of the viral genome from the essential viral packaging functions. These viral vector components are normally provided to the production cells on separate DNA expression cassettes (alternatively known as plasmids, expression plasmids, DNA constructs or expression constructs).

The vector genome comprises the NOI. Vector genomes typically require a packaging signal (ip), the internal expression cassette harbouring the NOI, (optionally) a post-transcriptional element (PRE), typically a central polypurine tract (cppt), the 3′-ppu and a self-inactivating (SIN) LTR. The R-U5 regions are required for correct polyadenylation of both the vector genome RNA and NOI mRNA, as well as the process of reverse transcription. The vector genome may optionally include an open reading frame, as described in WO 2003/064665, which allows for vector production in the absence of rev.

The packaging functions include the gag/pol and env genes. These are required for the production of vector particles by the production cell. Providing these functions in trans to the genome facilitates the production of replication-defective viral vectors.

Production systems for gamma-retroviral vectors are typically 3-component systems requiring genome, gag/pol and env expression constructs. Production systems for HIV-1-based lentiviral vectors may additionally require the accessory gene rev to be provided and for the vector genome to include the rev-responsive element (RRE). EIAV-based lentiviral vectors do not require rev to be provided in trans if an open-reading frame (ORF) is present within the genome (see WO 2003/064665).

Usually both the “external” promoter (which drives the vector genome cassette) and “internal” promoter (which drives the NOI cassette) encoded within the vector genome cassette are strong eukaryotic or virus promoters, as are those driving the other vector system components. Examples of such promoters include CMV, EF1a, PGK, CAG, TK, SV40 and Ubiquitin promoters. Strong ‘synthetic’ promoters, such as those generated by DNA libraries (e.g. JeT promoter) may also be used to drive transcription. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human al-antitrypsin (hAAT) promoter, phosphoenolpyruvate carboxykinase (PEPCK), liver fatty acid binding protein promoter, Flt-1 promoter, INF-13 promoter, Mb promoter, SP-B promoter, SYN1 promoter, WASP promoter, SV40/hAlb promoter, SV40/CD43, SV40/CD45, NSE/RU5′ promoter, ICAM-2 promoter, GPIIb promoter, GFAP promoter, Fibronectin promoter, Endoglin promoter, Elastase-1 promoter, Desmin promoter, CD68 promoter, CD14 promoter and B29 promoter may be used to drive transcription.

Production of retroviral vectors involves either the transient co-transfection of the production cells with these DNA components or use of stable production cell lines wherein all the components are stably integrated within the production cell genome (e.g. Stewart H J, Fong-Wong L, Strickland I, Chipchase D, Kelleher M, Stevenson L, Thoree V, McCarthy J, Ralph G S, Mitrophanous K A and Radcliffe P A. (2011). Hum Gene Ther. March; 22 (3):357-69). An alternative approach is to use a stable packaging cell (into which the packaging components are stably integrated) and then transiently transfect in the vector genome plasmid as required (e.g. Stewart, H. J., M. A. Leroux-Carlucci, C. J. Sion, K. A. Mitrophanous and P. A. Radcliffe (2009). Gene Ther. June; 16 (6):805-14). It is also feasible that alternative, not complete, packaging cell lines could be generated (just one or two packaging components are stably integrated into the cell lines) and to generate vector the missing components are transiently transfected. The production cell may also express regulatory proteins such as a member of the tet repressor (TetR) protein group of transcription regulators (e.g. T-Rex, Tet-On, and Tet-Off), a member of the cumate inducible switch system group of transcription regulators (e.g. cumate repressor (CymR) protein), or an RNA-binding protein (e.g. TRAP—tryptophan-activated RNA-binding protein).

In one embodiment of the present invention, the viral vector is derived from EIAV. EIAV has the simplest genomic structure of the lentiviruses and is particularly preferred for use in the present invention. In addition to the gag/pol and env genes, EIAV encodes three other genes: tat, rev, and S2. Tat acts as a transcriptional activator of the viral LTR (Derse and Newbold (1993) Virology 194(2):530-536 and Maury et al (1994) Virology 200(2):632-642) and rev regulates and coordinates the expression of viral genes through rev-response elements (RRE) (Martarano et al. (1994) J Virol 68(5):3102-3111). The mechanisms of action of these two proteins are thought to be broadly similar to the analogous mechanisms in the primate viruses (Martarano et al. (1994) J Virol 68(5):3102-3111). The function of S2 is unknown. In addition, an EIAV protein, Ttm, has been identified that is encoded by the first exon of tat spliced to the env coding sequence at the start of the transmembrane protein. In an alternative embodiment of the present invention the viral vector is derived from HIV: HIV differs from EIAV in that it does not encode S2 but unlike EIAV it encodes vif, vpr, vpu and nef.

The term “recombinant retroviral or lentiviral vector” (RRV) refers to a vector with sufficient retroviral genetic information to allow packaging of an RNA genome, in the presence of packaging components, into a viral particle capable of transducing a target cell. Transduction of the target cell may include reverse transcription and integration into the target cell genome. The RRV carries non-viral coding sequences which are to be delivered by the vector to the target cell. A RRV is incapable of independent replication to produce infectious retroviral particles within the target cell. Usually the RRV lacks a functional gag/pol and/or env gene, and/or other genes essential for replication.

Preferably the RRV vector of the present invention has a minimal viral genome.

As used herein, the term “minimal viral genome” means that the viral vector has been manipulated so as to remove the non-essential elements whilst retaining the elements essential to provide the required functionality to infect, transduce and deliver a NOI to a target cell. Further details of this strategy can be found in WO 1998/17815 and WO 99/32646. A minimal EIAV vector lacks tat, rev and S2 genes and neither are these genes provided in trans in the production system. A minimal HIV vector lacks vif, vpr, vpu, tat and nef.

The expression plasmid used to produce the vector genome within a production cell may include transcriptional regulatory control sequences operably linked to the retroviral genome to direct transcription of the genome in a production cell/packaging cell. All 3rd generation lentiviral vectors are deleted in the 5′ U3 enhancer-promoter region, and transcription of the vector genome RNA is driven by heterologous promoter such as another viral promoter, for example the CMV promoter, as discussed below. This feature enables vector production independently of tat. Some lentiviral vector genomes require additional sequences for efficient virus production. For example, particularly in the case of HIV, RRE sequences may be included. However the requirement for RRE on the (separate) GagPol cassette (and dependence on rev which is provided in trans) may be reduced or eliminated by codon optimisation of the GagPol ORF. Further details of this strategy can be found in WO 2001/79518.

Alternative sequences which perform the same function as the rev/RRE system are also known. For example, a functional analogue of the rev/RRE system is found in the Mason Pfizer monkey virus. This is known as the constitutive transport element (CTE) and comprises an RRE-type sequence in the genome which is believed to interact with a factor in the infected cell. The cellular factor can be thought of as a rev analogue. Thus, CTE may be used as an alternative to the rev/RRE system. Any other functional equivalents of the Rev protein which are known or become available may be relevant to the invention. For example, it is also known that the Rex protein of HTLV-I can functionally replace the Rev protein of HIV-1. Rev and RRE may be absent or non-functional in the vector for use in the methods of the present invention; in the alternative rev and RRE, or functionally equivalent system, may be present.

As used herein, the term “functional substitute” means a protein or sequence having an alternative sequence which performs the same function as another protein or sequence. The term “functional substitute” is used interchangeably with “functional equivalent” and “functional analogue” herein with the same meaning.

SIN Vectors

The lentiviral vectors as described herein may be used in a self-inactivating (SIN) configuration in which the viral enhancer and promoter sequences have been deleted. SIN vectors can be generated and transduce non-dividing target cells in vivo, ex vivo or in vitro with an efficacy similar to that of non-SIN vectors. The transcriptional inactivation of the long terminal repeat (LTR) in the SIN provirus should prevent mobilisation of vRNA, and is a feature that further diminishes the likelihood of formation of replication-competent virus. This should also enable the regulated expression of genes from internal promoters by eliminating any cis-acting effects of the LTR.

By way of example, self-inactivating retroviral vector systems have been constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus. However, any promoter(s) internal to the LTRs in such vectors will still be transcriptionally active. This strategy has been employed to eliminate effects of the enhancers and promoters in the viral LTRs on transcription from internally placed genes. Such effects include increased transcription or suppression of transcription. This strategy can also be used to eliminate downstream transcription from the 3′ LTR into genomic DNA. This is of particular concern in human gene therapy where it is important to prevent the adventitious activation of any endogenous oncogene. Yu et al., (1986) PNAS 83: 3194-98; Marty et al., (1990) Biochimie 72: 885-7; Naviaux et al., (1996) J. Virol. 70: 5701-5; Iwakuma et al., (1999) Virol. 261: 120-32; Deglon et al., (2000) Human Gene Therapy 11: 179-90. SIN lentiviral vectors are described in U.S. Pat. Nos. 6,924,123 and 7,056,699.

Replication-Defective Lentiviral Vectors

In the genome of a replication-defective lentiviral vector the sequences of gag/pol and/or env may be mutated and/or not functional.

In a typical lentiviral vector as described herein, at least part of one or more coding regions for proteins essential for virus replication may be removed from the vector. This makes the viral vector replication-defective. Portions of the viral genome may also be replaced by a NOI in order to generate a vector comprising an NOI which is capable of transducing a non-dividing target cell and/or integrating its genome into the target cell genome.

In one embodiment the lentiviral vectors are non-integrating vectors as described in WO 2006/010834 and WO 2007/071994.

In a further embodiment the vectors have the ability to deliver a sequence which is devoid of or lacking viral RNA. In a further embodiment a heterologous binding domain (heterologous to gag) located on the RNA to be delivered and a cognate binding domain on Gag or GagPol can be used to ensure packaging of the RNA to be delivered. Both of these vectors are described in WO 2007/072056.

NOI and Polynucleotides

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to those of skill in the art. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing PCR under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

Common Retroviral Vector Elements

Promoters and Enhancers

Expression of a NOI and polynucleotide may be controlled using control sequences for example transcription regulation elements or translation repression elements, which include promoters, enhancers and other expression regulation signals (e.g. tet repressor (TetR) system) or the Transgene Repression In vector Production cell system (TRiP) or other regulators of NOIs described herein.

Prokaryotic promoters and promoters functional in eukaryotic cells may be used. Tissue-specific or stimuli-specific promoters may be used. Chimeric promoters may also be used comprising sequence elements from two or more different promoters.

Suitable promoting sequences are strong promoters including those derived from the genomes of viruses, such as polyoma virus, adenovirus, fowlpox virus, bovine papilloma virus, avian sarcoma virus, cytomegalovirus (CMV), retrovirus and Simian Virus 40 (SV40), or from heterologous mammalian promoters, such as the actin promoter, EF1a, CAG, TK, SV40, ubiquitin, PGK or ribosomal protein promoter. Alternatively, tissue-specific promoters such as rhodopsin (Rho), rhodopsin kinase (RhoK), cone-rod homeobox containing gene (CRX), neural retina-specific leucine zipper protein (NRL), Vitelliform Macular Dystrophy 2 (VMD2), Tyrosine hydroxylase, neuronal-specific neuronal-specific enolase (NSE) promoter, astrocyte-specific glial fibrillary acidic protein (GFAP) promoter, human al-antitrypsin (hAAT) promoter, phosphoenolpyruvate carboxykinase (PEPCK), liver fatty acid binding protein promoter, Flt-1 promoter, INF-8 promoter, Mb promoter, SP-B promoter, SYN1 promoter, WASP promoter, SV40/hAlb promoter, SV40/CD43, SV40/CD45, NSE/RU5′ promoter, ICAM-2 promoter, GPIIb promoter, GFAP promoter, Fibronectin promoter, Endoglin promoter, Elastase-1 promoter, Desmin promoter, CD68 promoter, CD14 promoter and B29 promoter may be used to drive transcription.

Transcription of a NOI may be increased further by inserting an enhancer sequence into the vector. Enhancers are relatively orientation- and position-independent; however, one may employ an enhancer from a eukaryotic cell virus, such as the SV40 enhancer and the CMV early promoter enhancer. The enhancer may be spliced into the vector at a position 5′ or 3′ to the promoter, but is preferably located at a site 5′ from the promoter.

The promoter can additionally include features to ensure or to increase expression in a suitable target cell. For example, the features can be conserved regions e.g. a Pribnow Box or a TATA box. The promoter may contain other sequences to affect (such as to maintain, enhance or decrease) the levels of expression of a nucleotide sequence. Suitable other sequences include the Sh1-intron or an ADH intron. Other sequences include inducible elements, such as temperature, chemical, light or stress inducible elements. Also, suitable elements to enhance transcription or translation may be present.

Regulators of NOIs

A complicating factor in the generation of retroviral packaging/producer cell lines and retroviral vector production is that constitutive expression of certain retroviral vector components and NOIs are cytotoxic leading to death of cells expressing these components and therefore inability to produce vector. Therefore, the expression of these components (e.g. gag-pol and envelope proteins such as VSV-G) can be regulated. The expression of other non-cytotoxic vector components, e.g. rev, can also be regulated to minimise the metabolic burden on the cell. The modular constructs and/or cells as described herein may comprise cytotoxic and/or non-cytotoxic vector components associated with at least one regulatory element. As used herein, the term “regulatory element” refers to any element capable of affecting, either increasing or decreasing, the expression of an associated gene or protein. A regulatory element includes a gene switch system, transcription regulation element and translation repression element.

A number of prokaryotic regulator systems have been adapted to generate gene switches in mammalian cells. Many retroviral packaging and producer cell lines have been controlled using gene switch systems (e.g. tetracycline and cumate inducible switch systems) thus enabling expression of one or more of the retroviral vector components to be switched on at the time of vector production. Gene switch systems include those of the (TetR) protein group of transcription regulators (e.g. T-Rex, Tet-On, and Tet-Off), those of the cumate inducible switch system group of transcription regulators (e.g. CymR protein) and those involving an RNA-binding protein (e.g. TRAP).

One such tetracycline-inducible system is the tetracycline repressor (TetR) system based on the T-REx™ system. By way of example, in such a system tetracycline operators (TetO2) are placed in a position such that the first nucleotide is 10 bp from the 3′ end of the last nucleotide of the TATATAA element of the human cytomegalovirus major immediate early promoter (hCMVp) then TetR alone is capable of acting as a repressor (Yao F, Svensjo T, Winkler T, Lu M, Eriksson C, Eriksson E. Tetracycline repressor, tetR, rather than the tetR-mammalian cell transcription factor fusion derivatives, regulates inducible gene expression in mammalian cells. 1998. Hum Gene Ther, 9: 1939-1950). In such a system the expression of the NOI can be controlled by a CMV promoter into which two copies of the TetO2 sequence have been inserted in tandem. TetR homodimers, in the absence of an inducing agent (tetracycline or its analogue doxycycline [dox]), bind to the TetO2 sequences and physically block transcription from the upstream CMV promoter. When present, the inducing agent binds to the TetR homodimers, causing allosteric changes such that it can no longer bind to the TetO2 sequences, resulting in gene expression. The TetR gene may be codon optimised as this may improve translation efficiency resulting in tighter control of TetO2 controlled gene expression.

The TRiP system is described in WO 2015/092440 and provides another way of repressing expression of the NOI in the production cells during vector production. The TRAP-binding sequence (e.g. TRAP-tbs) interaction forms the basis for a transgene protein repression system for the production of retroviral vectors, when a constitutive and/or strong promoter, including a tissue-specific promoter, driving the transgene is desirable and particularly when expression of the transgene protein in production cells leads to reduction in vector titres and/or elicits an immune response in vivo due to viral vector delivery of transgene-derived protein (Maunder et al, Nat Commun. (2017) March 27; 8).

Briefly, the TRAP-tbs interaction forms a translational block, repressing translation of the transgene protein (Maunder et al, Nat Commun. (2017) March 27; 8). The translational block is only effective in production cells and as such does not impede the DNA- or RNA-based vector systems. The TRiP system is able to repress translation when the transgene protein is expressed from a constitutive and/or strong promoter, including a tissue-specific promoter from single- or multi cistronic mRNA. It has been demonstrated that unregulated expression of transgene protein can reduce vector titres and affect vector product quality. Repression of transgene protein for both transient and stable PaCL/PCL vector production systems is beneficial for production cells to prevent a reduction in vector titres: where toxicity or molecular burden issues may lead to cellular stress; where transgene protein elicits an immune response in vivo due to viral vector delivery of transgene-derived protein; where the use of gene-editing transgenes may result in on/off target affects; where the transgene protein may affect vector and/or envelope glycoprotein exclusion.

Envelope and Pseudotyping

In one preferred aspect, the lentiviral vector as described herein has been pseudotyped. In this regard, pseudotyping can confer one or more advantages. For example, the env gene product of the HIV based vectors would restrict these vectors to infecting only cells that express a protein called CD4. But if the env gene in these vectors has been substituted with env sequences from other enveloped viruses, then they may have a broader infectious spectrum (Verma and Somia (1997) Nature 389(6648):239-242). By way of example, workers have pseudotyped an HIV based vector with the glycoprotein from VSV (Verma and Somia (1997) Nature 389(6648):239-242).

In another alternative, the Env protein may be a modified Env protein such as a mutant or engineered Env protein. Modifications may be made or selected to introduce targeting ability or to reduce toxicity or for another purpose (Valsesia-Wittman et al 1996 J Virol 70: 2056-64; Nilson et al (1996) Gene Ther 3(4):280-286; and Fielding et al (1998) Blood 91(5):1802-1809 and references cited therein).

The vector may be pseudotyped with any molecule of choice.

As used herein, “env” shall mean an endogenous lentiviral envelope or a heterologous envelope, as described herein.

VSV-G

The envelope glycoprotein (G) of Vesicular stomatitis virus (VSV), a rhabdovirus, is an envelope protein that has been shown to be capable of pseudotyping certain enveloped viruses and viral vector virions.

Its ability to pseudotype MoMLV-based retroviral vectors in the absence of any retroviral envelope proteins was first shown by Emi et al. (1991) Journal of Virology 65:1202-1207. WO 1994/294440 teaches that retroviral vectors may be successfully pseudotyped with VSV-G. These pseudotyped VSV-G vectors may be used to transduce a wide range of mammalian cells. More recently, Abe et al. (1998) J Virol 72(8) 6356-6361 teach that non-infectious retroviral particles can be made infectious by the addition of VSV-G.

Burns et al. (1993) Proc. Natl. Acad. Sci. USA 90:8033-7 successfully pseudotyped the retrovirus MLV with VSV-G and this resulted in a vector having an altered host range compared to MLV in its native form. VSV-G pseudotyped vectors have been shown to infect not only mammalian cells, but also cell lines derived from fish, reptiles and insects (Burns et al. (1993) ibid). They have also been shown to be more efficient than traditional amphotropic envelopes for a variety of cell lines (Yee et al., (1994) Proc. Natl. Acad. Sci. USA 91:9564-9568, Emi et al. (1991) Journal of Virology 65:1202-1207). VSV-G protein can be used to pseudotype certain retroviruses because its cytoplasmic tail is capable of interacting with the retroviral cores.

The provision of a non-retroviral pseudotyping envelope such as VSV-G protein gives the advantage that vector particles can be concentrated to a high titre without loss of infectivity (Akkina et al. (1996) J. Virol. 70:2581-5). Retrovirus envelope proteins are apparently unable to withstand the shearing forces during ultracentrifugation, probably because they consist of two non-covalently linked subunits. The interaction between the subunits may be disrupted by the centrifugation. In comparison the VSV glycoprotein is composed of a single unit. VSV-G protein pseudotyping can therefore offer potential advantages for both efficient target cell infection/transduction and during manufacturing processes.

WO 2000/52188 describes the generation of pseudotyped retroviral vectors, from stable producer cell lines, having vesicular stomatitis virus-G protein (VSV-G) as the membrane-associated viral envelope protein, and provides a gene sequence for the VSV-G protein.

Ross River Virus

The Ross River viral envelope has been used to pseudotype a non-primate lentiviral vector (FIV) and following systemic administration predominantly transduced the liver (Kang et al., 2002, J. Virol., 76:9378-9388). Efficiency was reported to be 20-fold greater than obtained with VSV-G pseudotyped vector, and caused less cytotoxicity as measured by serum levels of liver enzymes suggestive of hepatotoxicity.

Baculovirus GP64

The baculovirus GP64 protein has been shown to be an alternative to VSV-G for viral vectors used in the large-scale production of high-titre virus required for clinical and commercial applications (Kumar M, Bradow B P, Zimmerberg J (2003) Hum Gene Ther. 14(1):67-77). Compared with VSV-G-pseudotyped vectors, GP64-pseudotyped vectors have a similar broad tropism and similar native titres. Because, GP64 expression does not kill cells, HEK293T-based cell lines constitutively expressing GP64 can be generated.

Alternative Envelopes

Other envelopes which give reasonable titre when used to pseudotype EIAV include Mokola, Rabies, Ebola and LCMV (lymphocytic choriomeningitis virus). Intravenous infusion into mice of lentivirus pseudotyped with 4070A led to maximal gene expression in the liver.

Packaging Sequence

As utilized within the context of the present invention the term “packaging signal”, which is referred to interchangeably as “packaging sequence” or “psi”, is used in reference to the non-coding, cis-acting sequence required for encapsidation of retroviral RNA strands during viral particle formation. In HIV-1, this sequence has been mapped to loci extending from upstream of the major splice donor site (SD) to at least the gag start codon (some or all of the 5′ sequence of gag to nucleotide 688 may be included). In EIAV the packaging signal comprises the R region into the 5′ coding region of Gag.

As used herein, the term “extended packaging signal” or “extended packaging sequence” refers to the use of sequences around the psi sequence with further extension into the gag gene. The inclusion of these additional packaging sequences may increase the efficiency of insertion of vector RNA into viral particles.

Feline immunodeficiency virus (FIV) RNA encapsidation determinants have been shown to be discrete and non-continuous, comprising one region at the 5′ end of the genomic mRNA (R-U5) and another region that mapped within the proximal 311 nt of gag (Kaye et al., J Virol. October; 69(10):6588-92 (1995).

Internal Ribosome Entry Site (IRES)

Insertion of IRES elements allows expression of multiple coding regions from a single promoter (Adam et al (as above); Koo et al (1992) Virology 186:669-675; Chen et al 1993 J. Virol 67:2142-2148). IRES elements were first found in the non-translated 5′ ends of picornaviruses where they promote cap-independent translation of viral proteins (Jang et al (1990) Enzyme 44: 292-309). When located between open reading frames in an RNA, IRES elements allow efficient translation of the downstream open reading frame by promoting entry of the ribosome at the IRES element followed by downstream initiation of translation.

A review on IRES is presented by Mountford and Smith (TIG May 1995 vol 11, No 5:179-184). A number of different IRES sequences are known including those from encephalomyocarditis virus (EMCV) (Ghattas, I. R., et al., Mol. Cell. Biol., 11:5848-5859 (1991); BiP protein [Macejak and Sarnow, Nature 353:91 (1991)]; the Antennapedia gene of Drosophila (exons d and e) [Oh, et al., Genes & Development, 6:1643-1653 (1992)] as well as those in polio virus (PV) [Pelletier and Sonenberg, Nature 334: 320-325 (1988); see also Mountford and Smith, TIG 11, 179-184 (1985)].

IRES elements from PV, EMCV and swine vesicular disease virus have previously been used in retroviral vectors (Coffin et al, as above).

The term “IRES” includes any sequence or combination of sequences which work as or improve the function of an IRES. The IRES(s) may be of viral origin (such as EMCV IRES, PV IRES, or FMDV 2A-like sequences) or cellular origin (such as FGF2 IRES, NRF IRES, Notch 2 IRES or EIF4 IRES).

In order for the IRES to be capable of initiating translation of each polynucleotide it should be located between or prior to the polynucleotides in the modular construct.

The nucleotide sequences utilised for development of stable cell lines require the addition of selectable markers for selection of cells where stable integration has occurred. These selectable markers can be expressed as a single transcription unit within the nucleotide sequence or it may be preferable to use IRES elements to initiate translation of the selectable marker in a polycistronic message (Adam et al 1991 J. Virol. 65, 4985).

Genetic Orientation and Insulators

It is well known that nucleic acids are directional and this ultimately affects mechanisms such as transcription and replication in the cell. Thus genes can have relative orientations with respect to one another when part of the same nucleic acid construct.

In certain embodiments of the present invention, at least two nucleic acid sequences present at the same locus in the cell or construct can be in a reverse and/or alternating orientations. In other words, in certain embodiments of the invention at this particular locus, the pair of sequential genes will not have the same orientation. This can help prevent both transcriptional and translational read-through when the region is expressed within the same physical location of the host cell.

Having the alternating orientations benefits retroviral vector production when the nucleic acids required for vector production are based at the same genetic locus within the cell. This in turn can also improve the safety of the resulting constructs in preventing the generation of replication-competent retroviral vectors.

When nucleic acid sequences are in reverse and/or alternating orientations the use of insulators can prevent inappropriate expression or silencing of a NOI from its genetic surroundings.

The term “insulator” refers to a class of DNA sequence elements that when bound to insulator-binding proteins possess an ability to protect genes from surrounding regulator signals. There are two types of insulators: an enhancer blocking function and a chromatin barrier function. When an insulator is situated between a promoter and an enhancer, the enhancer-blocking function of the insulator shields the promoter from the transcription-enhancing influence of the enhancer (Geyer and Corces 1992; Kellum and Schedl 1992). The chromatin barrier insulators function by preventing the advance of nearby condensed chromatin which would lead to a transcriptionally active chromatin region turning into a transcriptionally inactive chromatin region and resulting in silencing of gene expression. Insulators which inhibit the spread of heterochromatin, and thus gene silencing, recruit enzymes involved in histone modifications to prevent this process (Yang J, Corces V G. 2011; 110:43-76; Huang, Li et al. 2007; Dhillon, Raab et al. 2009). An insulator can have one or both of these functions and the chicken β-globin insulator (cHS4) is one such example. This insulator is the most extensively studied vertebrate insulator, is highly rich in G+C and has both enhancer-blocking and heterochromatic barrier functions (Chung J H, Whitely M, Felsenfeld G. Cell. 1993; 74:505-514). Other such insulators with enhancer blocking functions are not limited to but include the following: human β-globin insulator 5 (HS5), human β-globin insulator 1 (HS1), and chicken β-globin insulator (cHS3) (Farrell CM1, West A G, Felsenfeld G., Mol Cell Biol. 2002 June; 22(11):3820-31; J Ellis et al. EMBO J. 1996 Feb. 1; 15(3): 562-568). In addition to reducing unwanted distal interactions the insulators also help to prevent promoter interference (i.e. where the promoter from one transcription unit impairs expression of an adjacent transcription unit) between adjacent retroviral nucleic acid sequences. If the insulators are used between each of the retroviral vector nucleic acid sequences, then the reduction of direct read-through will help prevent the formation of replication-competent retroviral vector particles.

The insulator may be present between each of the retroviral nucleic acid sequences. In one embodiment, the use of insulators prevents promoter-enhancer interactions from one NOI expression cassette interacting with another NOI expression cassette in a nucleotide sequence encoding vector components.

An insulator may be present between the vector genome and gag-pol sequences. This therefore limits the likelihood of the production of a replication-competent retroviral vector and ‘wild-type’ like RNA transcripts, improving the safety profile of the construct. The use of insulator elements to improve the expression of stably integrated multigene vectors is cited in Moriarity et al, Nucleic Acids Res. 2013 April; 41(8):e92.

Vector Titre

The skilled person will understand that there are a number of different methods of determining the titre of lentiviral vectors. Titre is often described as transducing units/mL (TU/mL). Titre may be increased by increasing the number of vector particles and by increasing the specific activity of a vector preparation.

Therapeutic Use

The lentiviral vector as described herein or a cell or tissue transduced with the lentiviral vector as described herein may be used in medicine.

In addition, the lentiviral vector as described herein, a production cell of the invention or a cell or tissue transduced with the lentiviral vector as described herein may be used for the preparation of a medicament to deliver a nucleotide of interest to a target site in need of the same. Such uses of the lentiviral vector or transduced cell of the invention may be for therapeutic or diagnostic purposes, as described previously.

Accordingly, there is provided a cell transduced by the lentiviral vector as described herein.

A “cell transduced by a viral vector particle” is to be understood as a cell, in particular a target cell, into which the nucleic acid carried by the viral vector particle has been transferred.

In one embodiment of the invention, the nucleotide of interest is translated in a target cell which lacks TRAP.

“Target cell” is to be understood as a cell in which it is desired to express the NOI. The NOI may be introduced into the target cell using a viral vector of the present invention. Delivery to the target cell may be performed in vivo, ex vivo or in vitro.

In a preferred embodiment, the nucleotide of interest gives rise to a therapeutic effect.

The NOI may have a therapeutic or diagnostic application. Suitable NOIs include, but are not limited to sequences encoding enzymes, co-factors, cytokines, chemokines, hormones, antibodies, anti-oxidant molecules, engineered immunoglobulin-like molecules, single chain antibodies, fusion proteins, immune co-stimulatory molecules, immunomodulatory molecules, chimeric antigen receptors a transdomain negative mutant of a target protein, toxins, conditional toxins, antigens, transcription factors, structural proteins, reporter proteins, subcellular localization signals, tumour suppressor proteins, growth factors, membrane proteins, receptors, vasoactive proteins and peptides, anti-viral proteins and ribozymes, and derivatives thereof (such as derivatives with an associated reporter group). The NOIs may also encode micro-RNA. Without wishing to be bound by theory, it is believed that the processing of micro-RNA will be inhibited by TRAP.

In one embodiment, the NOI may be useful in the treatment of a neurodegenerative disorder.

In another embodiment, the NOI may be useful in the treatment of Parkinson's disease.

In another embodiment, the NOI may encode an enzyme or enzymes involved in dopamine synthesis. For example, the enzyme may be one or more of the following: tyrosine hydroxylase, GTP-cyclohydrolase I and/or aromatic amino acid dopa decarboxylase. The sequences of all three genes are available (GenBank® Accession Nos. X05290, U19523 and M76180, respectively).

In another embodiment, the NOI may encode the vesicular monoamine transporter 2 (VMAT2). In an alternative embodiment the viral genome may comprise a NOI encoding aromatic amino acid dopa decarboxylase and a NOI encoding VMAT2. Such a genome may be used in the treatment of Parkinson's disease, in particular in conjunction with peripheral administration of L-DOPA.

In another embodiment the NOI may encode a therapeutic protein or combination of therapeutic proteins.

In another embodiment, the NOI may encode a protein or proteins selected from the group consisting of glial cell derived neurotophic factor (GDNF), brain derived neurotrophic factor (BDNF), ciliary neurotrophic factor (CNTF), neurotrophin-3 (NT-3), acidic fibroblast growth factor (aFGF), basic fibroblast growth factor (bFGF), interleukin-1 beta (IL-1β), tumor necrosis factor alpha (TNF-α), insulin growth factor-2, VEGF-A, VEGF-B, VEGF-C/VEGF-2, VEGF-D, VEGF-E, PDGF-A, PDGF-B, hetero- and homo-dimers of PDFG-A and PDFG-B.

In another embodiment, the NOI may encode an anti-angiogenic protein or anti-angiogenic proteins selected from the group consisting of angiostatin, endostatin, platelet factor 4, pigment epithelium derived factor (PEDF), placental growth factor, restin, interferon-α, interferon-inducible protein, gro-beta and tubedown-1, interleukin (IL)-1, IL-12, retinoic acid, anti-VEGF antibodies or fragments/variants thereof such as aflibercept, thrombospondin, VEGF receptor proteins such as those described in U.S. Pat. Nos. 5,952,199 and 6,100,071, and anti-VEGF receptor antibodies.

In another embodiment, the NOI may encode anti-inflammatory proteins, antibodies or fragment/variants of proteins or antibodies selected from the group consisting of NF-kB inhibitors, IL1beta inhibitors, TGFbeta inhibitors, IL-6 inhibitors, IL-23 inhibitors, IL-18 inhibitors, Tumour necrosis factor alpha and Tumour necrosis factor beta, Lymphotoxin alpha and Lymphotoxin beta, LIGHT inhibitors, alpha synuclein inhibitors, Tau inhibitors, beta amyloid inhibitors, IL-17 inhibitors,

In another embodiment the NOI may encode cystic fibrosis transmembrane conductance regulator (CFTR).

In another embodiment the NOI may encode a protein normally expressed in an ocular cell.

In another embodiment, the NOI may encode a protein normally expressed in a photoreceptor cell and/or retinal pigment epithelium cell.

In another embodiment, the NOI may encode a protein selected from the group comprising RPE65, arylhydrocarbon-interacting receptor protein like 1 (AIPL1), CRB1, lecithin retinal acetyltransferace (LRAT), photoreceptor-specific homeo box (CRX), retinal guanylate cyclise (GUCY2D), RPGR interacting protein 1 (RPGRIP1), LCA2, LCA3, LCA5, dystrophin, PRPH2, CNTF, ABCR/ABCA4, EMP1, TIMP3, MERTK, ELOVL4, MYO7A, USH2A, VMD2, RLBP1, COX-2, FPR, harmonin, Rab escort protein 1, CNGB2, CNGA3, CEP 290, RPGR, RS1, RP1, PRELP, glutathione pathway enzymes and opticin.

In other embodiments, the NOI may encode the human clotting Factor VIII or Factor IX.

In other embodiments, the NOI may encode protein or proteins involved in metabolism selected from the group comprising phenylalanine hydroxylase (PAH), Methylmalonyl CoA mutase, Propionyl CoA carboxylase, Isovaleryl CoA dehydrogenase, Branched chain ketoacid dehydrogenase complex, Glutaryl CoA dehydrogenase, Acetyl CoA carboxylase, propionyl CoA carboxylase, 3 methyl crotonyl CoA carboxylase, pyruvate carboxylase, carbamoyl-phophate synthase ammonia, ornithine transcarbamylase, glucosylceramidase beta, alpha galactosidase A, glucosylceramidase beta, cystinosin, glucosamine (N-acetyl)-6-sulfatase, N-acetyl-alpha-glucosaminidase, N-sulfoglucosamine sulfohydrolase, Galactosamine-6 sulfatase, arylsulfatase A, cytochrome B-245 beta, ABCD1, ornithine carbamoyltransferase, argininosuccinate synthase, argininosuccinate lysase, arginase 1, alanine glycoxhylate amino transferase, ATP-binding cassette, sub-family B members.

In other embodiments, the NOI may encode a chimeric antigen receptor (CAR) or a T cell receptor (TCR). In one embodiment, the CAR is an anti-5T4 CAR. In other embodiments, the NOI may encode B-cell maturation antigen (BCMA), CD19, CD22, CD20, CD138, CD30, CD33, CD123, CD70, prostate specific membrane antigen (PSMA), Lewis Y antigen (LeY), Tyrosine-protein kinase transmembrane receptor (ROR1), Mucin 1, cell surface associated (Muc1), Epithelial cell adhesion molecule (EpCAM), endothelial growth factor receptor (EGFR), insulin, protein tyrosine phosphatase, non-receptor type 22, interleukin 2 receptor, alpha, interferon induced with helicase C domain 1, human epidermal growth factor receptor (HER2), glypican 3 (GPC3), disialoganglioside (GD2), mesiothelin, vesicular endothelial growth factor receptor 2 (VEGFR2).

In other embodiments, the NOI may encode a chimeric antigen receptor (CAR) against NKG2D ligands selected from the group comprising ULBP1, 2 and 3, H60, Rae-1a, b, g, d, MICA, MICB.

In further embodiments the NOI may encode SGSH, SUMF1, GAA, the common gamma chain (CD132), adenosine deaminase, WAS protein, globins, alpha galactosidase A, 6-aminolevulinate (ALA) synthase, 6-aminolevulinate dehydratase (ALAD), Hydroxymethylbilane (HMB) synthase, Uroporphyrinogen (URO) synthase, Uroporphyrinogen (URO) decarboxylase, Coproporphyrinogen (COPRO) oxidase, Protoporphyrinogen (PROTO) oxidase, Ferrochelatase, α-L-iduronidase, Iduronate sulfatase, Heparan sulfamidase, N-acetylglucosaminidase, Heparan-α-glucosaminide N-acetyltransferase, 3 N-acetylglucosamine 6-sulfatase, Galactose-6-sulfate sulfatase, β-galactosidase, N-acetylgalactosamine-4-sulfatase, β-glucuronidase and Hyaluronidase.

In addition to the NOI the vector may also comprise or encode a siRNA, shRNA, or regulated shRNA. (Dickins et al. (2005) Nature Genetics 37: 1289-1295, Silva et al. (2005) Nature Genetics 37:1281-1288).

Indications

The vectors, including retroviral and AAV vectors, according to the present invention may be used to deliver one or more NOI(s) useful in the treatment of the disorders listed in WO 1998/05635, WO 1998/07859, WO 1998/09985. The nucleotide of interest may be DNA or RNA. Examples of such diseases are given below:
A disorder which responds to cytokine and cell proliferation/differentiation activity; immunosuppressant or immunostimulant activity (e.g. for treating immune deficiency, including infection with human immunodeficiency virus, regulation of lymphocyte growth; treating cancer and many autoimmune diseases, and to prevent transplant rejection or induce tumour immunity); regulation of haematopoiesis (e.g. treatment of myeloid or lymphoid diseases); promoting growth of bone, cartilage, tendon, ligament and nerve tissue (e.g. for healing wounds, treatment of burns, ulcers and periodontal disease and neurodegeneration); inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); anti-inflammatory activity (for treating, for example, septic shock or Crohn's disease); macrophage inhibitory and/or T cell inhibitory activity and thus, anti-inflammatory activity; anti-immune activity (i.e. inhibitory effects against a cellular and/or humoral immune response, including a response not associated with inflammation); inhibition of the ability of macrophages and T cells to adhere to extracellular matrix components and fibronectin, as well as up-regulated fas receptor expression in T cells.
Malignancy disorders, including cancer, leukaemia, benign and malignant tumour growth, invasion and spread, angiogenesis, metastases, ascites and malignant pleural effusion.
Autoimmune diseases including arthritis, including rheumatoid arthritis, hypersensitivity, allergic reactions, asthma, systemic lupus erythematosus, collagen diseases and other diseases.
Vascular diseases including arteriosclerosis, atherosclerotic heart disease, reperfusion injury, cardiac arrest, myocardial infarction, vascular inflammatory disorders, respiratory distress syndrome, cardiovascular effects, peripheral vascular disease, migraine and aspirin-dependent anti-thrombosis, stroke, cerebral ischaemia, ischaemic heart disease or other diseases.
Diseases of the gastrointestinal tract including peptic ulcer, ulcerative colitis, Crohn's disease and other diseases.
Hepatic diseases including hepatic fibrosis, liver cirrhosis.
Inherited metabolic disorders including phenylketonuria PKU, Wilson disease, organic acidemias, urea cycle disorders, cholestasis, and other diseases.
Renal and urologic diseases including thyroiditis or other glandular diseases, glomerulonephritis or other diseases.
Ear, nose and throat disorders including otitis or other oto-rhino-laryngological diseases, dermatitis or other dermal diseases.
Dental and oral disorders including periodontal diseases, periodontitis, gingivitis or other dental/oral diseases.
Testicular diseases including orchitis or epididimo-orchitis, infertility, orchidal trauma or other testicular diseases.
Gynaecological diseases including placental dysfunction, placental insufficiency, habitual abortion, eclampsia, pre-eclampsia, endometriosis and other gynaecological diseases.
Ophthalmologic disorders such as Leber Congenital Amaurosis (LCA) including LCA10, posterior uveitis, intermediate uveitis, anterior uveitis, conjunctivitis, chorioretinitis, uveoretinitis, optic neuritis, glaucoma, including open angle glaucoma and juvenile congenital glaucoma, intraocular inflammation, e.g. retinitis or cystoid macular oedema, sympathetic ophthalmia, scleritis, retinitis pigmentosa, macular degeneration including age related macular degeneration (AMD) and juvenile macular degeneration including Best Disease, Best vitelliform macular degeneration, Stargardt's Disease, Usher's syndrome, Doyne's honeycomb retinal dystrophy, Sorby's Macular Dystrophy, Juvenile retinoschisis, Cone-Rod Dystrophy, Corneal Dystrophy, Fuch's Dystrophy, Leber's congenital amaurosis, Leber's hereditary optic neuropathy (LHON), Adie syndrome, Oguchi disease, degenerative fondus disease, ocular trauma, ocular inflammation caused by infection, proliferative vitreo-retinopathies, acute ischaemic optic neuropathy, excessive scarring, e.g. following glaucoma filtration operation, reaction against ocular implants, corneal transplant graft rejection, and other ophthalmic diseases, such as diabetic macular oedema, retinal vein occlusion, RLBP1-associated retinal dystrophy, choroideremia and achromatopsia.
Neurological and neurodegenerative disorders including Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, strokes, post-polio syndrome, psychiatric disorders, myelitis, encephalitis, subacute sclerosing pan-encephalitis, encephalomyelitis, acute neuropathy, subacute neuropathy, chronic neuropathy, Fabry disease, Gaucher disease, Cystinosis, Pompe disease, metachromatic leukodystrophy, Wiscott Aldrich Syndrome, adrenoleukodystrophy, beta-thalassemia, sickle cell disease, Guillaim-Barre syndrome, Sydenham chorea, myasthenia gravis, pseudo-tumour cerebri, Down's Syndrome, Huntington's disease, CNS compression or CNS trauma or infections of the CNS, muscular atrophies and dystrophies, diseases, conditions or disorders of the central and peripheral nervous systems, motor neuron disease including amyotropic lateral sclerosis, spinal muscular atropy, spinal cord and avulsion injury.
Other diseases and conditions such as cystic fibrosis, mucopolysaccharidosis including Sanfilipo syndrome A, Sanfilipo syndrome B, Sanfilipo syndrome C, Sanfilipo syndrome D, Hunter syndrome, Hurler-Scheie syndrome, Morquio syndrome, ADA-SCID, X-linked SCID, X-linked chronic granulomatous disease, porphyria, haemophilia A, haemophilia B, post-traumatic inflammation, haemorrhage, coagulation and acute phase response, cachexia, anorexia, acute infection, septic shock, infectious diseases, diabetes mellitus, complications or side effects of surgery, bone marrow transplantation or other transplantation complications and/or side effects, complications and side effects of gene therapy, e.g. due to infection with a viral carrier, or AIDS, to suppress or inhibit a humoral and/or cellular immune response, for the prevention and/or treatment of graft rejection in cases of transplantation of natural or artificial cells, tissue and organs such as cornea, bone marrow, organs, lenses, pacemakers, natural or artificial skin tissue.

siRNA, micro-RNA and shRNA

In certain other embodiments, the NOI comprises a micro-RNA. Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

In addition to the NOI the vector may also comprise or encode a siRNA, shRNA, or regulated shRNA (Dickins et al. (2005) Nature Genetics 37: 1289-1295, Silva et al. (2005) Nature Genetics 37:1281-1288).

Post-transcriptional gene silencing (PTGS) mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nature Medicine 11:429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA >30 bp has been found to activate the interferon response leading to shut-down of protein synthesis and non-specific mRNA degradation (Stark et al., Annu Rev Biochem 67:227-64 (1998)). However this response can be bypassed by using 21 nt siRNA duplexes (Elbashir et al., EMBO J. December 3; 20(23):6877-88 (2001), Hutvagner et al., Science. August 3, 293(5531):834-8. Eupub July 12 (2001)) allowing gene function to be analysed in cultured mammalian cells.

Pharmaceutical Compositions

The present disclosure provides a pharmaceutical composition comprising the lentiviral vector as described herein or a cell or tissue transduced with the viral vector as described herein, in combination with a pharmaceutically acceptable carrier, diluent or excipient.

The present disclosure provides a pharmaceutical composition for treating an individual by gene therapy, wherein the composition comprises a therapeutically effective amount of a lentiviral vector. The pharmaceutical composition may be for human or animal usage.

The composition may comprise a pharmaceutically acceptable carrier, diluent, excipient or adjuvant. The choice of pharmaceutical carrier, excipient or diluent can be made with regard to the intended route of administration and standard pharmaceutical practice. The pharmaceutical compositions may comprise, or be in addition to, the carrier, excipient or diluent any suitable binder(s), lubricant(s), suspending agent(s), coating agent(s), solubilising agent(s) and other carrier agents that may aid or increase vector entry into the target site (such as for example a lipid delivery system).

Where appropriate, the composition can be administered by any one or more of inhalation; in the form of a suppository or pessary; topically in the form of a lotion, solution, cream, ointment or dusting powder; by use of a skin patch; orally in the form of tablets containing excipients such as starch or lactose, or in capsules or ovules either alone or in admixture with excipients, or in the form of elixirs, solutions or suspensions containing flavouring or colouring agents; or they can be injected parenterally, for example intracavernosally, intravenously, intramuscularly, intracranially, intraoccularly intraperitoneally, or subcutaneously. For parenteral administration, the compositions may be best used in the form of a sterile aqueous solution which may contain other substances, for example enough salts or monosaccharides to make the solution isotonic with blood. For buccal or sublingual administration, the compositions may be administered in the form of tablets or lozenges which can be formulated in a conventional manner.

The lentiviral vector as described herein may also be used to transduce target cells or target tissue ex vivo prior to transfer of said target cell or tissue into a patient in need of the same. An example of such cell may be autologous T cells and an example of such tissue may be a donor cornea.

Variants, Derivatives, Analogues, Homologues and Fragments

In addition to the specific proteins and nucleotides mentioned herein, the present invention also encompasses the use of variants, derivatives, analogues, homologues and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” means an entity having a certain homology with the wild type amino acid sequence and the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 97 or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95%, 97%, 98% or 99% identical to the subject sequence. Although homology can also be considered in terms of similarity, in the context of the present invention it is preferred to express homology in terms of sequence identity.

Homology comparisons can be conducted by eye, or more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Research 12:387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol Lett (1999) 174(2):247-50; FEMS Microbiol Lett (1999) 177(1):187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate percentage homology, preferably percentage sequence identity. The software usually does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the break. The DNA is then expressed in accordance with the invention to make the encoded protein. These methods are only illustrative of the numerous standard techniques known in the art for manipulation of DNA sequences and other known techniques may also be used.

All variants, fragments or homologues of the regulatory protein suitable for use in the cells and/or modular constructs of the invention will retain the ability to bind the cognate binding site of the NOI such that translation of the NOI is repressed or prevented in a viral vector production cell.

All variants fragments or homologues of the binding site will retain the ability to bind the cognate RNA-binding protein, such that translation of the NOI is repressed or prevented in a viral vector production cell.

Codon Optimisation

The polynucleotides used in the present invention (including the NOI and/or components of the vector production system) may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including retroviruses, use a large number of rare codons and changing these to correspond to commonly used mammalian codons, increases expression of a gene of interest, e.g. a NOI or packaging components in mammalian production cells, can be achieved. Codon usage tables are known in the art for mammalian cells, as well as for a variety of other organisms.

Codon optimisation of viral vector components has a number of other advantages. By virtue of alterations in their sequences, the nucleotide sequences encoding the packaging components of the viral particles required for assembly of viral particles in the producer cells/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. In lentiviral vectors codon optimisation also overcomes the Rev/RRE requirement for export, rendering optimised sequences Rev-independent. Codon optimisation also reduces homologous recombination between different constructs within the vector system (for example between the regions of overlap in the gag-pol and env open reading frames). The overall effect of codon optimisation is therefore a notable increase in viral titre and improved safety.

In one embodiment only codons relating to INS are codon optimised. However, in a much more preferred and practical embodiment, the sequences are codon optimised in their entirety, with some exceptions, for example the sequence encompassing the frameshift site of gag-pol (see below).

The gag-pol gene of lentiviral vectors comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimised. Retaining this fragment will enable more efficient expression of the Gag-Pol proteins. For EIAV the beginning of the overlap has been taken to be nt 1262 (where nucleotide 1 is the A of the gag ATG) and the end of the overlap to be nt 1461. In order to ensure that the frameshift site and the gag-pol overlap are preserved, the wild type sequence has been retained from nt 1156 to 1465.

Derivations from optimal codon usage may be made, for example, in order to accommodate convenient restriction sites, and conservative amino acid changes may be introduced into the Gag-Pol proteins.

In one embodiment, codon optimisation is based on lightly expressed mammalian genes. The third and sometimes the second and third base may be changed.

Due to the degenerate nature of the genetic code, it will be appreciated that numerous gag-pol sequences can be achieved by a skilled worker. Also there are many retroviral variants described which can be used as a starting point for generating a codon-optimised gag-pol sequence.

Lentiviral genomes can be quite variable. For example there are many quasi-species of HIV-1 which are still functional. This is also the case for EIAV. These variants may be used to enhance particular parts of the transduction process. Examples of HIV-1 variants may be found at the HIV Databases operated by Los Alamos National Security, LLC at http://hiv-web.lanl.gov. Details of EIAV clones may be found at the National Center for Biotechnology Information (NCBI) database located at http://www.ncbi.nlm.nih.gov.

The strategy for codon-optimised gag-pol sequences can be used in relation to any retrovirus. This would apply to all lentiviruses, including EIAV, FIV, BIV, CAEV, VMR, SIV, HIV-1 and HIV-2. In addition this method could be used to increase expression of genes from HTLV-1, HTLV-2, HFV, HSRV and human endogenous retroviruses (HERV), MLV and other retroviruses.

Codon optimisation can render gag-pol expression Rev-independent. In order to enable the use of anti-rev or RRE factors in the lentiviral vector, however, it would be necessary to render the viral vector generation system totally Rev/RRE-independent. Thus, the genome also needs to be modified. This is achieved by optimising vector genome components. Advantageously, these modifications also lead to the production of a safer system absent of all additional proteins both in the producer and in the transduced cell.

Combination with Modified U1 to Improve Vector Titre

MSD-mutated lentiviral vectors are preferable to current standard lentiviral vectors for use as gene therapy vectors due to their reduced capacity to partake in aberrant splicing events both during LV production and in target cells. However, until the present invention, the production of MSD-mutated vectors has either relied upon supply of the HIV-1 tat protein (1^{st} and 2^{nd} generation U3-dependent lentiviral vectors) or has been of lower efficiency due to the unstabilising effect of mutating the MSD on vector RNA levels (in 3^{rd} generation vectors). Due to safety reasons there is no desire or justification for ‘reintroducing’ tat back into contemporary 3^{rd} generation, U3-independent LV systems, and consequently there is currently no solution to the reduction in production titres of MSD-mutated vectors intended for clinical use.

The present inventors show that MSD-mutated, 3^{rd} generation (i.e. U3/tat-independent) LVs can be produced to high titre by co-expression of a modified U1 snRNA directed to bind to the 5′packaging region of the vector genome RNA during production. It is surprisingly shown that these modified U1 snRNAs can enhance the production titres of MSD-mutated LVs in a manner that is independent of the presence of the 5′polyA signal within the 5′R region, indicating a novel mechanism over others' use of modified U1 snRNAs to suppress polyadenylation (so called U1-interference, [Ui]). It is surprisingly shown that targeting the modified U1 snRNAs to critical sequences of the packaging region produce the greatest enhancement in MSD-mutated LV titres. The present inventors also disclose novel sequence mutation within the major splice donor region such that reduction in titres of MSD-mutated LV is less pronounced, and that enhancement in titres of such MSD-mutated LV variants by modified U1 snRNAs is greatest.

The present inventors have surprisingly found that the output titres of lentiviral vectors can be enhanced by co-expressing non-coding RNAs based on U1 snRNAs, which have been modified so that they no longer target the endogenous sequence (a splice donor site) but now target a sequence within the vRNA molecule. As demonstrated in the present Examples, the inventors show that the relative enhancement in output titres of lentiviral vectors harbouring attenuating mutations within the major splice donor region (containing the major splice donor and cryptic splice donor sites) by said modified U1 snRNAs are greater than standard lentiviral vectors containing a non-mutated major splice donor region.

As demonstrated in the present Examples, vector genomes harbouring a broad range of mutation types within the major splice donor region (point mutations, region deletion, and sequence replacement) that lead to reduced titres may be used in combination with a modified U1 snRNA. The approach may comprise co-expression of modified U1 snRNAs together with the other vector components during vector production. The modified U1 snRNAs are designed such that binding to the consensus splice donor site has been ablated by replacing it with a heterologous sequence that is complementary to a target sequence within the vector genome vRNA. The invention describes various modes of application and optimal characteristics of the modified U1 snRNAs, including target sequence and complementarity length, design and modes of expression.

In one aspect of the invention as described herein, the vector may be used in combination with a modified U1 snRNA. This is discussed further below.

The presence/abundance of modified U1 snRNA molecule can be quantified within vector production cell extracts or vector virions by extraction of total RNA followed by RT-PCR or RT-qPCR (quantitative) using DNA primers. Importantly, the forward primer is designed such that is has complementarity to the targeting sequence of the modified U1 snRNA molecule so that only the modified U1 snRNA is amplified during qPCR and not endogenous U1 snRNA.

In one aspect the present invention provides a vector virion comprising a modified U1 according to the present invention as described herein.

Splicing and polyadenylation are key processes for mRNA maturation, particularly in higher eukaryotes where most protein-coding transcripts contain multiple introns. The elements within a pre-mRNA that are required for splicing include the 5′ splice donor signal, the sequence surrounding the branch point and the 3′ splice acceptor signal. Interacting with these three elements is the spliceosome, which is formed by five small nuclear RNAs (snRNAs), including U1 snRNA, and associated nuclear proteins (snRNP). U1 snRNA is expressed by a polymerase II promoter and is present in most eukaryotic cells (Lund et al., 1984, J. Biol. Chem., 259:2013-2021). Human U1 snRNA (small nuclear RNA) is 164 nt long with a well-defined structure consisting of four stem-loops (West, S., 2012, Biochemical Society Transactions, 40:846-849). U1 snRNA contains a short sequence at its 5′-end that is broadly complementary to the 5′ splice donor sites at exon-intron junctions. U1 snRNA participates in splice-site selection and spliceosome assembly by base pairing to the 5′ splice donor site. A known function for U1 snRNA outside splicing is in the regulation of 3′-end mRNA processing: it suppresses premature polyadenylation (polyA) at early polyA signals (particularly within introns).

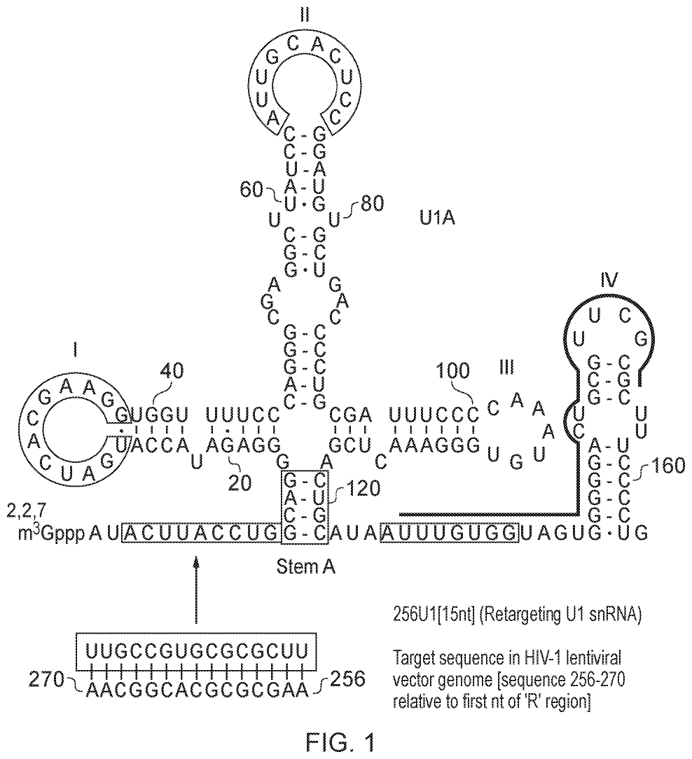

Human U1 snRNA (small nuclear RNA) is 164 nt long with a well-defined structure consisting of four stem-loops (see FIG. 1). The endogenous non-coding RNA, U1 snRNA, binds to the consensus 5′ splice donor site (e.g. 5′-MAGGURR-3′, wherein M is A or C and R is A or G) via the native splice donor annealing sequence (e.g. 5′-ACUUACCUG-3′) during early steps of intron splicing. Stem loop I binds to U1A-70K protein that has been shown to be important for polyA suppression. Stem loop II binds to U1A protein, and the 5′-AUUUGUGG-3′ sequence binds to Sm proteins, which together with Stem loop IV, is important for U1 snRNA processing. As defined herein the modified U1 snRNA for use according to the present invention is modified to introduce a heterologous sequence that is complementary to a target sequence within the vector genome vRNA molecule at the site of the native splice donor targeting/annealing sequence (see FIG. 1).

As used herein, the terms “modified U1 snRNA”, “re-directed U1 snRNA”, “re-targeted U1 snRNA”, “re-purposed U1 snRNA” and “mutant U1 snRNA”, mean a U1 snRNA that has been modified so that it is no longer complementary to the consensus 5′ splice donor site sequence (e.g. 5′-MAGGURR-3′) that it uses to initiate the splicing process of a target gene. Thus, a modified U1 snRNA is a U1 snRNA which has been modified so that it is no longer complementary to the splice donor site sequence (e.g. 5′-MAGGURR-3′). Instead, the modified U1 snRNA is designed so that it targets or is complementary to a nucleotide sequence having a unique RNA sequence within the packaging region of a MSD-mutated lentiviral vector genome molecule (target site), i.e. a sequence that is unrelated to splicing of the vRNA. The nucleotide sequence within the packaging region of a MSD-mutated lentiviral vector genome molecule can be preselected. Thus, the modified U1 snRNA is a U1 snRNA which has been modified so that its 5′ end is complementary to a nucleotide sequence within the packaging region of a MSD-mutated lentiviral vector genome molecule. As a result, and not wishing to be bound by theory, the modified U1 snRNA is thought to bind to the target site sequence based on complementarity of the target site sequence with the short sequence at the 5′ end of the modified U1 snRNA, thus stabilising the vRNA leading to increased output vector titres of the MSD-mutated lentiviral vector.

As used herein, the terms “native splice donor annealing sequence” and “native splice donor targeting sequence” mean the short sequence at the 5′-end of the endogenous U1 snRNA that is broadly complementary to the consensus 5′ splice donor site of introns. The native splice donor annealing sequence may be 5′-ACUUACCUG-3′.

As used herein, the term “consensus 5′ splice donor site” means the consensus RNA sequence at the 5′ end of introns used in splice-site selection, e.g. having the sequence 5′-MAGGURR-3′.

As used herein, the terms “nucleotide sequence within the packaging region of a MSD-mutated lentiviral vector genome sequence”, “target sequence” and “target site” mean a site having a particular RNA sequence within the packaging region of a MSD-mutated lentiviral vector genome molecule which has been preselected as the target site for binding/annealing the modified U1 snRNA.

As used herein, the terms “packaging region of a MSD-mutated lentiviral vector genome molecule” and “packaging region of an MSD-mutated lentiviral vector genome sequence” means the region at the 5′ end of an MSD-mutated lentiviral vector genome from the beginning of the 5′ U5 domain to the terminus of the sequence derived from gag gene. Thus, the packaging region of a MSD-mutated lentiviral vector genome molecule includes the 5′ U5 domain, PBS element, stem loop (SL) 1 element, SL2 element, SL3ip element, SL4 element and the sequence derived from the gag gene. It is common in the art to provide the complete gag gene in trans to the genome during lentiviral vector production to enable the production of replication-defective viral vector particle. The nucleotide sequence of the gag gene provided in trans need not be encoded by wild type nucleotides but may be codon-optimised; importantly the chief attribute of the gag gene provided in trans is that it encodes and directs expression of the gag and gagpol proteins. Accordingly, it will be understood by the person skilled in the art that, if the complete gag gene is to be provided in trans during lentiviral vector production, the term “packaging region of a lentiviral vector genome molecule” may mean the region at the 5′ end of the MSD-mutated lentiviral vector genome molecule from the beginning of the 5′ U5 domain through to the ‘core’ packaging signal at the SL3 ii element, and the native gag nucleotide sequence from the ATG codon (present within SL4) to the end of the remaining gag nucleotide sequence present on the vector genome.

As used herein, the term “sequence derived from gag gene” means, any native sequence of the gag gene derived from the ATG codon to nucleotide 688 (Kharytonchyk, S. et. al., 2018, J. Mol. Biol., 430:2066-79) that may be present, e.g. remain, in the vector genome.

As used herein, the terms “to introduce within the first 11 nucleotides of the U1 snRNA, which encompasses the native splice donor annealing sequence, a heterologous sequence”, “to introduce within the nine nucleotides at positions 3-to-11 said heterologous sequence” and “to introduce within the first 11 nucleotides at the 5′ end of the U1 snRNA a heterologous sequence” include to replace the first 11 nucleotides, or the nine nucleotides at positons 3-to-11, of the U1 snRNA all or in part with said heterologous sequence or to modify the first 11 nucleotides, or the nine nucleotides at positons 3-to-11, of the U1 snRNA to have the same sequence as said heterologous sequence.

As used herein, the terms “to introduce within the native splice donor annealing sequence a heterologous sequence” and “to introduce within the native splice donor annealing sequence at the 5′ end of the U1 snRNA a heterologous sequence” include to replace the native splice donor annealing sequence all or in part with said heterologous sequence or to modify the native splice donor annealing sequence to have the same sequence as said heterologous sequence.

As used herein, the term “enhances lentiviral vector titres” includes “increases lentiviral vector titres”, “recovers lentiviral vector titres” and “improves lentiviral vector titres”.

Accordingly, in one embodiment, the modified U1 snRNA has been modified to bind to a nucleotide sequence within the packaging region of an MSD-mutated lentiviral vector genome sequence.

In some embodiments, the modified U1 snRNA is modified at the 5′ end relative to the endogenous U1 snRNA to introduce a heterologous sequence that is complementary to a nucleotide sequence within the packaging region of an MSD-mutated lentiviral vector genome.

In some embodiments, the modified U1 snRNA is modified at the 5′ end relative to the endogenous U1 snRNA to introduce within the native splice donor annealing sequence a heterologous sequence that is complementary to a nucleotide sequence within the the packaging region of an MSD-mutated lentiviral vector genome.

The modified U1 snRNA may be modified at the 5′ end relative to the endogenous U1 snRNA to replace a sequence encompassing the native splice donor annealing sequence with a heterologous sequence that is complementary to said nucleotide sequence.

The modified U1 snRNA may be a modified U1 snRNA variant. The U1 snRNA variant which is modified in accordance with the invention may be a naturally occurring U1 snRNA variant, a U1 snRNA variant containing a mutation within the stem loop I region ablating U1-70K protein binding, or a U1 snRNA variant containing a mutation in the stem loop II region ablating U1A protein binding. The U1 snRNA variant containing a mutation within the stem loop I region ablating U1-70K protein binding may be U1_m1 or U1_m2, preferably U1A_m1 or U1A_m2.

In some embodiments, the modified U1 snRNA as described herein comprises a nucleotide sequence having at least 70% identity (suitable at least 75%, at least 80%, 85%, 90%, 91%, 92%, 93%, 94%, 95%, 96%, 97%, 98%, 99% identity) with the main U1 snRNA sequence [clover leaf] (nt 410-562) of the U1_256 sequence as described herein. In some embodiments, the modified U1 snRNA of the invention comprises the main U1 snRNA sequence [clover leaf] (nt 410-562) of the U1_256 sequence as described herein. The main U1 snRNA sequence [clover leaf] (nt 410-562) of the U1_256 sequence (SEQ ID NO:15) is as follows:

(SEQ ID NO: 66)
GCAGGGGAGATACCATGATCACGAAGGTGGTTTTCCCAGGGCGAGG
CTTATCCATTGCACTCCGGATGTGCTGACCCCTGCGATTTCCCCAA
ATGTGGGAAACTCGACTGCATAATTTGTGGTAGTGGGGGACTGCGT
TCGCGCTTTCCCCTG.

In some preferred embodiments, the first 11 nucleotides of the U1 snRNA, which encompasses the native splice donor annealing sequence, may be all or in part replaced with a heterologous sequence that is complementary to a nucleotide sequence within the packaging region of an MSD-mutated lentiviral vector genome. Suitably, 1-11 (suitably 2-11, 3-11, 1, 2, 3, 4, 5, 6, 7, 8, 9, 10 or 11), nucleic acids of the first 11 nucleotides of the U1 snRNA are replaced with a heterologous sequence that is complementary to a nucleotide sequence within the packaging region of an MSD-mutated lentiviral vector genome.

In some embodiments, the native splice donor annealing sequence, may be all or in part replaced with a heterologous sequence that is complementary to a nucleotide sequence within the packaging region of an MSD-mutated lentiviral vector genome. Suitably, 1-11 (suitably 2-11, 3-11, 5-11, 1, 2, 3, 4, 5, 6, 7, 8, 9, 10 or 11), nucleic acids of the native splice donor annealing sequence are replaced with a heterologous sequence that is complementary to a nucleotide sequence within the packaging region of an MSD-mutated lentiviral vector genome. In a preferred embodiment, the entire native splice donor annealing sequence is replaced with a heterologous sequence that is complementary to a nucleotide sequence within the packaging region of an MSD-mutated lentiviral vector genome, i.e. the native splice donor annealing sequence (e.g. 5′-ACUUACCUG-3′) is fully replaced with a heterologous sequence as described herein.

In some embodiments, a heterologous sequence that is complementary to a nucleotide sequence within the packaging region of an MSD-mutated lentiviral vector genome comprises at least 7 nucleotides of complementarity to said nucleotide sequence. In some embodiments, a heterologous sequence that is complementary to a nucleotide sequence within the packaging region of an MSD-mutated lentiviral vector genome comprises at least 9 nucleotides of complementarity to said nucleotide sequence. Preferably, a heterologous sequence for use in the present invention comprises 15 nucleotides of complementarity to said nucleotide sequence.

Suitably, a heterologous sequence for use in the present invention may comprise 7-25 (suitably 7-20, 7-15, 9-15, 7, 8, 9, 10, 11, 12, 13, 14, 15, 16, 17, 18, 19, 20, 21, 22, 23, 24 or 25) nucleotides.

Suitably, a heterologous sequence for use in the present invention may comprise 25 nucleotides.

In some embodiments, the nucleotide sequence within the packaging region of an MSD-mutated lentiviral vector genome is located within the 5′ U5 domain, PBS element, SL1 element, SL2 element, SL3ip element, SL4 element and/or the sequence derived from gag gene. Suitably, the nucleotide sequence within the packaging region of an MSD-mutated lentiviral vector genome is located within the SL1, SL2 and/or SL3ip element(s). In some preferred embodiments, the nucleotide sequence within the packaging region of an MSD-mutated lentiviral vector genome is located within the SL1 and/or SL2 element(s). In some particularly preferred embodiments, the nucleotide sequence within the packaging region of an MSD-mutated lentiviral vector genome is located within the SL1 element.

In some embodiments, a nucleotide sequence within the packaging region of an MSD-mutated lentiviral vector genome comprises at least 7 nucleotides. In some embodiments, a nucleotide sequence within the packaging region of an MSD-mutated lentiviral vector genome comprises at least 9 nucleotides. Suitably, a nucleotide sequence within the packaging region of an MSD-mutated lentiviral vector genome comprises 7-25 (suitably 7-20, 7-15, 9-15, 7, 8, 9, 10, 11, 12, 13, 14, or 15) nucleotides.

Preferably, a nucleotide sequence within the packaging region of an MSD-mutated lentiviral vector genome comprises 15 nucleotides.

The binding of a modified U1 snRNA as described herein to the nucleotide sequence within the packaging region of an MSD-mutated lentiviral vector genome may enhance lentiviral vector titre during lentiviral vector production relative to lentiviral vector production in the absence of a modified U1 snRNA as described herein. Thus, production of a lentiviral vector in the presence of a modified U1 snRNA as described herein enhances lentiviral vector titre relative to lentiviral vector production in the absence of a modified U1 snRNA as described herein. A suitable assay for the measurement of lentiviral vector titre is as described herein. Suitably, the lentiviral vector production involves co-expression of said modified U1 snRNA with vector components including gag, env, rev and the RNA genome of the lentiviral vector. The RNA genome of the lentiviral vector may be an MSD-2KO RNA genome. In some embodiments, the enhancement of lentiviral vector titre occurs in the presence or absence of a functional 5′LTR polyA site. In some embodiments, the enhancement of lentiviral vector titres mediated by a modified U1 snRNA of the invention is independent of polyA site suppression in the 5′LTR of the vector genome.

In some embodiments, the binding of a modified U1 snRNA as described herein to the nucleotide sequence within the packaging region of an MSD-mutated lentiviral vector genome may increase lentiviral vector titre during lentiviral vector production by at least 30% relative to lentiviral vector production in the absence of a modified U1 snRNA as described herein. Suitably, the binding of a modified U1 snRNA as described herein to the nucleotide sequence within the packaging region of an MSD-mutated lentiviral vector genome may increase MSD-mutated lentiviral vector titre during production by at least 35% (suitably at least 40%, 45%, 50%, 60%, 70%, 100%, 150%, 200%, 250%, 300%, 350%, 400%, 450%, 500%, 550%, 600%, 650%, 700%, 750%, 800%, 850%, 900%, 950%, 1,000%, 2,000%, 5,000%, or 10,000%) relative to MSD-mutated lentiviral vector production in the absence of a modified U1 snRNA as described herein.

The modified U1 snRNAs as described herein may be designed by (a) selecting a target site in the packaging region of an MSD-mutated lentiviral vector genome for binding the modified U1 snRNA (the preselected nucleotide site); and (b) introducing within the native splice donor annealing sequence (e.g. 5′-ACUUACCUG-3′) at the 5′ end of the U1 snRNA a heterologous sequence that is complementary to the preselected nucleotide site selected in step (a).

The introduction of a heterologous sequence that is complementary to the target site within, or in place of, the native splice donor annealing sequence (e.g. 5′-ACUUACCUG-3′) at the 5′ end of the endogenous U1 snRNA using conventional techniques in molecular biology is within the capabilities of a person of ordinary skill in the art. Generally speaking, suitable routine methods include directed mutagenesis or replacement via homologous recombination.

The modification of the native splice donor annealing sequence (e.g. 5′-ACUUACCUG-3′) at the 5′ end of the endogenous U1 snRNA to have the same sequence as a heterologous sequence that is complementary to the target site using conventional techniques in molecular biology is within the capabilities of a person of ordinary skill in the art. For example, suitable methods include directed mutagenesis or random mutagenesis followed by selection for mutations which provide a modified U1 snRNA as described herein.

The modified U1 snRNAs as described herein can be manufactured according to methods generally known in the art. For example, the modified U1 snRNAs can be manufactured by chemical synthesis or recombinant DNA/RNA technology.

In one aspect the nucleotide sequence encoding a modified U1 snRNA may be on a different nucleotide sequence, for example on a different plasmid.

The introduction of a nucleotide sequence encoding a modified U1 snRNA as described herein into a cell using conventional molecular and cell biology techniques is within the capabilities of a person of ordinary skill in the art.

Combination with Improved TRAP Binding Site and Kozak Sequence

As discussed above, the TRIP system is described in WO 2015/092440 and provides a way of repressing expression of the NOI in the production cells during vector production. The TRAP-binding sequence (e.g. TRAP-tbs) interaction forms the basis for a transgene protein repression system for the production of retroviral vectors, when a constitutive and/or strong promoter, including a tissue-specific promoter, driving the transgene is desirable and particularly when expression of the transgene protein in production cells leads to reduction in vector titres and/or elicits an immune response in vivo due to viral vector delivery of transgene-derived protein (Maunder et al, Nat Commun. (2017) March 27; 8).

In one embodiment, the nucleotide sequence further comprises a tryptophan RNA-binding attenuation protein (TRAP) binding site or a portion thereof.

In one aspect the present invention provides a nucleic acid sequence comprising a nucleotide of interest (transgene) and a tryptophan RNA-binding attenuation protein (TRAP) binding site, wherein the TRAP binding site overlaps with the transgene start codon ATG.

Any disclosures herein relating to a Kozak sequence/overlapping Kozak sequence are equally applicable (where appropriate) to equivalent aspects referring to the ATG of the start codon and overlap therewith.

In another aspect, the present invention provides a nucleic acid sequence comprising a nucleotide of interest and a Kozak sequence, wherein said Kozak sequence comprises a portion of a tryptophan RNA-binding attenuation protein (TRAP) binding site.

In one aspect the present invention provides a nucleic acid sequence comprising a nucleotide of interest (transgene) and a TRAP binding site, wherein the TRAP binding site comprises a portion of the transgene start codon ATG or vice versa.

In one embodiment, the nucleotide sequence further comprises a tbs or a portion thereof as described herein, a multiple cloning site (MCS) and a Kozak sequence as described herein, wherein said MCS is located downstream of the tbs or portion thereof and upstream of the Kozak sequence. Suitably, the tbs or portion thereof and the Kozak sequence do not overlap.

In some embodiments of the present invention, the nucleotide of interest is operably linked to the tbs or the portion thereof. In some embodiments, the nucleotide of interest is translated in a target cell which lacks TRAP.

The tbs or the portion thereof may be capable of interacting with TRAP such that translation of the nucleotide of interest is repressed or prevented in a viral vector production cell.

Thus, provided is a method of repressing translation of a NOI in a viral vector production cell, the method comprising introducing into the viral vector production cell the nucleotide sequence of the invention and a nucleic acid sequence encoding a TRAP, wherein the TRAP binds to the TRAP binding site, or the portion thereof, thereby repressing translation of the NOI.

Table 1 shows sequences which may be used in the present invention, wherein K may be T or G, “R” is to be understood as specifying a purine (i.e. A or G) at that position in the sequence, “V” is to be understood as specifying any nucleotide from G, A, or C and “N” is to be understood as specifying any nucleotide at that position in the sequence. For example, this could be G, A, T, C or U. The TRAP binding site (tbs) sequence or 3′ tbs sequence is shown in italics, the multiple cloning site (MCS) is shown underlined, and the Kozak sequence is shown in bold.

TABLE 1

| SEQ | | |
|---|---|---|
| ID | | |
| NO | Description | Sequence |
| 69 | 3′ tbs-Kozak junction variant | GAGAATG |
| 70 | 3′ tbs-Kozak junction variant | GAGCATG |
| 71 | 3′ tbs-Kozak junction variant | GAGGATG |
| 72 | 3′ tbs-Kozak junction variant | TAGAATG |
| 73 | 3′ tbs-Kozak junction variant | TAGCATG |
| 74 | 3′ tbs-Kozak junction variant | TAGGATG |
| 75 | 3′ tbs-Kozak junction variant | GAGAAATG |
| 76 | 3′ tbs-Kozak junction variant | GAGACATG |
| 77 | 3′ tbs-Kozak junction variant | GAGAGATG |
| 78 | 3′ tbs-Kozak junction variant | GAGCAATG |
| 79 | 3′ tbs-Kozak junction variant | GAGCCATG |
| 80 | 3′ tbs-Kozak junction variant | GAGCGATG |
| 81 | 3′ tbs-Kozak junction variant | GAGGAATG |
| 82 | 3′ tbs-Kozak junction variant | GAGGCATG |
| 83 | 3′ tbs-Kozak junction variant | GAGGGATG |
| 84 | 3′ tbs-Kozak junction variant | TAGAAATG |
| 85 | 3′ tbs-Kozak junction variant | TAGACATG |
| 86 | 3′ tbs-Kozak junction variant | TAGAGATG |
| 87 | 3′ tbs-Kozak junction variant | TAGCAATG |
| 88 | 3′ tbs-Kozak junction variant | TAGCCATG |
| 89 | 3′ tbs-Kozak junction variant | TAGCGATG |
| 90 | 3′ tbs-Kozak junction variant | TAGGAATG |
| 91 | 3′ tbs-Kozak junction variant | TAGGCATG |
| 92 | 3′ tbs-Kozak junction variant | TAGGGATG |
| 93 | L33 Improved leader derived | CTTTTTCGCAACGGGTTTGCCGCCAGAACACAG |
| | from a splicing event | GTGTCGTGAAAA |
| 94 | TRAP from Bacillus subtilis | MNQKHSSDFVVIKAVEDGVNVIGLTRGTDTKFHHS |
| | | EKLDKGEVIIAQFTEHTSAIKVRGEALIQTAYGEMK |
| | | SEKK |
| 95 | TRAP from Aminomonas | MKEGEEAKTSVLSDYVVVKALENGVTVIGLTRGQE |
| | paucivorans | TKFAHTEKLDDGEVWIAQFTEHTSAIKVRGASEIHT |
| | | KHGMLFSGRGRNEKG |
| 96 | TRAP from | MNPMTDRSDITGDYVVVKALENGVTIIGLTRGGVT |
| | Desulfotomaculum | KFHHTEKLDKGEIMIAQFTEHTSAIKIRGRAELLTKH |
| | hydrothermale | GKIRTEVDS |
| 97 | TRAP from B. | MYTNSDFVVIKALEDGVNVIGLTRGADTRFHHSEK |
| | stearothermophilus | LDKGEVLIAQFTEHTSAIKVRGKAYIQTRHGVIESE |
| | | GKK |
| 98 | TRAP from B. | MYTNSDFVVIKALEDGVNVIGLTRGADTRFHHSEK |
| | stearothermophilus S72N | LDKGEVLIAQFTEHTSAIKVRGKAYIQTRHGVIENE |
| | | GKK |
| 99 | TRAP from B. halodurans | MNVGDNSNFFVIKAKENGVNVFGMTRGTDTRFHH |
| | | SEKLDKGEVMIAQFTEHTSAVKIRGKAIIQTSYGTL |
| | | DTEKDE |
| 100 | TRAP from | MVCDNFAFSSAINAEYIVVKALENGVTIMGLTRGKD |
| | Carboxydothermus | TKFHHTEKLDKGEVMVAQFTEHTSAIKIRGKAEIYT |
| | hydrogenoformans | KHGVIKNE |
| 101 | L33 Improved leader | CTTTTTCGCAACGGGTTTGCCGCCAGAACACAG |
| 102 | L12 Improved leader | CTTTTTCGCAAC |
| 103 | Extended Kozak consensus | GNNRVVATG |
| | sequence | |
| 104 | Core Kozak consensus | RVVATG |
| | sequence | |
| 105 | EMCV M loop | CGTGGTTTTCCTTTGAAAAACACGATGATACC |
| 106 | Optimal (overlapping) tbs- | GAGTTTAGCGGAGTGGAGAAGAGCGGAGCCGA |
| | Kozak | GCCTAGCAGAGACGAGCCGAGATG |
| 107 | Optimal (overlapping) tbs- | GAGTTTAGCGGAGTGGAGAAGAGCGGAGCCGA |
| | MCS-Kozak | GCCTAGCAGAGACGAGAAGAGCTCTAGACCATG |
| 108 | 3′ tbs-Kozak junction variant | TAGATG |
| 109 | 3′ tbs-Kozak junction variant | GAGATATG |
| 110 | 3′ tbs-Kozak junction variant | GAGCTATG |
| 111 | 3′ tbs-Kozak junction variant | TAGATATG |
| 112 | 3′ tbs-Kozak junction variant | TAGCTATG |
| 113 | 3′ tbs-Kozak consensus | KAGNNG |
| | sequence | |
| 114 | 3′ tbs-Kozak consensus | KAGATG |
| | sequence | |
| 115 | 3′ tbs-Kozak consensus | KAGNNTG |
| | sequence | |
| 116 | 3′ tbs-Kozak consensus | KAGNATG |
| | sequence | |
| 117 | Chicken β-Actin/Rabbit β- | CGGCGGGCGGGAACGTTGCCTTCGCCCCGTGC |
| | globin chimeric 5′UTR-intron | CCCGCTCCGCGCCGCCTCGCGCCGCCCGCCCC |
| | with tbs-kzkV0.G variant; | GGCTCTGACTGACCGCGTTACTCCCACAGGTG |
| | exonic sequence in bold | AGCGGGCGGGACGGCCCTTCTCCCTCCGGGCT |
| | (spliced together to become | GTAATTAGCGCTTGGTTTAATGACGGCTCGTTTC |
| | 5′UTR leader), tbskzkV0.G | TTTTCTGTGGCTGCGTGAAAGCCTTAAAGGGCT |
| | in italics | CCGGGAGGGCCTTTGTGCGGGGGGGAGCGGCT |
| | | CGGGGGGTGCGTGCGTGTGTGTGTGCGTGGGG |
| | | AGCGCCGCGTGCGGCCCGCGCTGCCCGGCGG |
| | | CTGTGAGCGCTGCGGGCGCGGCGCGGGGCTTT |
| | | GTGCGCTCCGCGTGTGCGCGAGGGGAGCGCG |
| | | GGCCGGGGGGGGTGCCCCGCGGTGCGGGGGG |
| | | GCTGCGAGGGGAACAAAGGCTGCGTGCGGGGT |
| | | GTGTGCGTGGGGGGGTGAGCAGGGGGTGTGG |
| | | GCGCGGCGGTCGGGCTGTAACCCCCCCCTGGC |
| | | ACCCCCCTCCCCGAGTTGCTGAGCACGGCCCG |
| | | GCTTCGGGTGCGGGGCTCCGTGCGGGGCGTGG |
| | | CGCGGGGCTCGCCGTGCCGGGCGGGGGGTGG |
| | | CGGCAGGTGGGGGTGCCGGGCGGGGCGGGGC |
| | | CGCCTCGGGCCGGGGAGGGCTCGGGGGAGGG |
| | | GCGCGGCGGCCCCGGAGCGCCGGCGGCTGTC |
| | | GAGGCGCGGCGAGCCGCAGCCATTGCCTTTTAT |
| | | GGTAATCGTGCGAGAGGGCGCAGGGACTTCCTT |
| | | TGTCCCAAATCTGGCGGAGCCGAAATCTGGGAG |
| | | GCGCCGCCGCACCCCCTCTAGCGGGCGCGGGC |
| | | GAAGCGGTGCGGCGCCGGCAGGAAGGAAATGG |
| | | GCGGGGAGGGCCTTCGTGCGTCGCCGCGCCGC |
| | | CGTCCCCTTCTCCATCTCCAGCCTCGGGGCTGC |
| | | CGCAGGGGGACGGCTGCCTTCGGGGGGGACG |
| | | GGGCAGGGGGGGGTTCGGCTTCTGGCGTGTGA |
| | | CCGGCGGCTTTAGAGCCTCTGCTAACCATGTTC |
| | | ATGCCTTCTTCTTTTTCCTACAGCTCCTGGGCAA |
| | | AGAGTTTAGCGGAGTGGAGAAGAGCGGAGCC |
| | | GAGCCTAGCAGAGACGAGCCGAGATG |
| 118 | EF1a 5′UTR-intron with | CTTTTTCGCAACGGGTTTGCCGCCAGAACACAG |
| | tbskzkV0.G variant; exonic | GTAAGTGCCGTGTGTGGTTCCCGCGGGCCTGG |
| | sequence in bold (spliced | CCTCTTTACGGGTTATGGCCCTTGCGTGCCTTG |
| | together to become 5′UTR | AATTACTTCCACCTGGCTGCAGTACGTGATTCTT |
| | leader), tbskzkV0.G in italics | GATCCCGAGCTTCGGGTTGGAAGTGGGTGGGA |
| | | GAGTTCGTGGCCTTGCGCTTAAGGAGCCCCTTC |
| | | GCCTCGTGCTTGAGTTGTGGCCTGGCCTGGGC |
| | | GCTGGGGCCGCCGCGTGCGAATCTGGTGGCAC |
| | | CTTCGCGCCTGTCTCGCTGCTTTCGATAAGTCTC |
| | | TAGCCATTTAAAATTTTTGATGACCTGCTGCGAC |
| | | GCTTTTTTTCTGGCAAGATAGTCTTGTAAATGCG |
| | | GGCCAAGATCAGCACACTGGTATTTCGGTTTTTG |
| | | GGGCCGCGGGCGGCGACGGGGCCCGTGCGTC |
| | | CCAGCGCACATGTTCGGCGAGGCGGGGCCTGC |
| | | GAGCGCGGCCACCGAGAATCGGACGGGGGTAG |
| | | TCTCAAGCTGCCCGGCCTGCTCTGGTGCCTGGC |
| | | CTCGCGCCGCCGTGTATCGCCCCGCCCTGGGC |
| | | GGCAAGGCTGGCCCGGTCGGCACCAGTTGCGT |
| | | GAGCGGAAAGATGGCCGCTTCCCGGCCCTGCT |
| | | GCAGGGAGCACAAAATGGAGGACGCGGCGCTC |
| | | GGGAGAGCGGGCGGGTGAGTCACCCACACAAA |
| | | GGAAAAGGGCCTTTCCGTCCTCAGCCGTCGCTT |
| | | CATGTGACTCCACGGAGTACCGGGCGCCGTCC |
| | | AGGCACCTCGATTAGTTCTCCAGCTTTTGGAGTA |
| | | CGTCGTCTTTAGGTTGGGGGGAGGGGTTTTATG |
| | | CGATGGAGTTTCCCCACACTGAGTGGGTGGAGA |
| | | CTGAAGTTAGGCCAGCTTGGCACTTGATGTAATT |
| | | CTCCTTGGAATTTGCCCTTTTTGAGTTTGGATCT |
| | | TGGTTCATTCTCAAGCCTCAGACAGTGGTTCAAA |
| | | GTTTTTTTCTTCCATTTCAGGTGTCGTGAAAAGA |
| | | GTTTAGCGGAGTGGAGAAGAGCGGAGCCGAG |
| | | CCTAGCAGAGACGAGCCGAGATG |
| 119 | Spliced sequence | CGGCGGGCGGGAACGTTGCCTTCGCCCCGTGC |
| | corresponding to SEQ ID | CCCGCTCCGCGCCGCCTCGCGCCGCCCGCCCC |
| | NO: 117; 5′UTR leader | GGCTCTGACTGACCGCGTTACTCCCACAGCTCC |
| | sequence in bold, | TGGGCAAAGAGTTTAGCGGAGTGGAGAAGAGC |
| | tbskzkV0.G in italics | GGAGCCGAGCCTAGCAGAGACGAGCCGAGAT |
| | | G |
| 120 | Spliced sequence | CTTTTTCGCAACGGGTTTGCCGCCAGAACACAG |
| | corresponding to SEQ ID | GTGTCGTGAAAAGAGTTTAGCGGAGTGGAGAA |
| | NO: 118; 5′UTR leader | GAGCGGAGCCGAGCCTAGCAGAGACGAGCCG |
| | sequence in bold, | AGATG |
| | tbskzkV0.G in italics | |
| 121 | Chicken β-Actin/Rabbit β- | CGGCGGGCGGGAACGTTGCCTTCGCCCCGTGC |
| | globin chimeric 5′UTR-intron | CCCGCTCCGCGCCGCCTCGCGCCGCCCGCCCC |
| | (exonic sequence in bold | GGCTCTGACTGACCGCGTTACTCCCACAGGTG |
| | (spliced together to become | AGCGGGGGGACGGCCCTTCTCCCTCCGGGCT |
| | 5′UTR leader)) | GTAATTAGCGCTTGGTTTAATGACGGCTCGTTTC |
| | | TTTTCTGTGGCTGCGTGAAAGCCTTAAAGGGCT |
| | | CCGGGAGGGCCTTTGTGCGGGGGGGAGCGGCT |
| | | CGGGGGGTGCGTGCGTGTGTGTGTGCGTGGGG |
| | | AGCGCCGCGTGCGGCCCGCGCTGCCCGGCGG |
| | | CTGTGAGCGCTGCGGGCGCGGCGCGGGGCTTT |
| | | GTGCGCTCCGCGTGTGCGCGAGGGGAGCGCG |
| | | GGCCGGGGGCGGTGCCCCGCGGTGCGGGGGG |
| | | GCTGCGAGGGGAACAAAGGCTGCGTGCGGGGT |
| | | GTGTGCGTGGGGGGGTGAGCAGGGGGTGTGG |
| | | GCGCGGCGGTCGGGCTGTAACCCCCCCCTGGC |
| | | ACCCCCCTCCCCGAGTTGCTGAGCACGGCCCG |
| | | GCTTCGGGTGCGGGGCTCCGTGCGGGGCGTGG |
| | | CGCGGGGCTCGCCGTGCCGGGGGGGGGGTGG |
| | | CGGCAGGTGGGGGTGCCGGGGGGGGCGGGGC |
| | | CGCCTCGGGCCGGGGAGGGCTCGGGGGAGGG |
| | | GCGCGGCGGCCCCGGAGCGCCGGCGGCTGTC |
| | | GAGGCGCGGCGAGCCGCAGCCATTGCCTTTTAT |
| | | GGTAATCGTGCGAGAGGGCGCAGGGACTTCCTT |
| | | TGTCCCAAATCTGGCGGAGCCGAAATCTGGGAG |
| | | GCGCCGCCGCACCCCCTCTAGCGGGCGCGGGC |
| | | GAAGCGGTGCGGCGCCGGCAGGAAGGAAATGG |
| | | GCGGGGAGGGCCTTCGTGCGTCGCCGCGCCGC |
| | | CGTCCCCTTCTCCATCTCCAGCCTCGGGGCTGC |
| | | CGCAGGGGGACGGCTGCCTTCGGGGGGGACG |
| | | GGGCAGGGGGGGGTTCGGCTTCTGGCGTGTGA |
| | | CCGGCGGCTTTAGAGCCTCTGCTAACCATGTTC |
| | | ATGCCTTCTTCTTTTTCCTACAGCTCCTGGGCAA |
| | | A |
| 122 | EF1a 5′UTR-intron (exonic | CTTTTTCGCAACGGGTTTGCCGCCAGAACACAG |
| | sequence in bold (spliced | GTAAGTGCCGTGTGTGGTTCCCGCGGGCCTGG |
| | together to become 5′UTR | CCTCTTTACGGGTTATGGCCCTTGCGTGCCTTG |
| | leader)) | AATTACTTCCACCTGGCTGCAGTACGTGATTCTT |
| | | GATCCCGAGCTTCGGGTTGGAAGTGGGTGGGA |
| | | GAGTTCGTGGCCTTGCGCTTAAGGAGCCCCTTC |
| | | GCCTCGTGCTTGAGTTGTGGCCTGGCCTGGGC |
| | | GCTGGGGCCGCCGCGTGCGAATCTGGTGGCAC |
| | | CTTCGCGCCTGTCTCGCTGCTTTCGATAAGTCTC |
| | | TAGCCATTTAAAATTTTTGATGACCTGCTGCGAC |
| | | GCTTTTTTTCTGGCAAGATAGTCTTGTAAATGCG |
| | | GGCCAAGATCAGCACACTGGTATTTCGGTTTTTG |
| | | GGGCCGCGGGCGGCGACGGGGCCCGTGCGTC |
| | | CCAGCGCACATGTTCGGCGAGGCGGGGCCTGC |
| | | GAGCGCGGCCACCGAGAATCGGACGGGGGTAG |
| | | TCTCAAGCTGCCCGGCCTGCTCTGGTGCCTGGC |
| | | CTCGCGCCGCCGTGTATCGCCCCGCCCTGGGC |
| | | GGCAAGGCTGGCCCGGTCGGCACCAGTTGCGT |
| | | GAGCGGAAAGATGGCCGCTTCCCGGCCCTGCT |
| | | GCAGGGAGCACAAAATGGAGGACGCGGCGCTC |
| | | GGGAGAGCGGGGGGTGAGTCACCCACACAAA |
| | | GGAAAAGGGCCTTTCCGTCCTCAGCCGTCGCTT |
| | | CATGTGACTCCACGGAGTACCGGGCGCCGTCC |
| | | AGGCACCTCGATTAGTTCTCCAGCTTTTGGAGTA |
| | | CGTCGTCTTTAGGTTGGGGGGAGGGGTTTTATG |
| | | CGATGGAGTTTCCCCACACTGAGTGGGTGGAGA |
| | | CTGAAGTTAGGCCAGCTTGGCACTTGATGTAATT |
| | | CTCCTTGGAATTTGCCCTTTTTGAGTTTGGATCT |
| | | TGGTTCATTCTCAAGCCTCAGACAGTGGTTCAAA |
| | | GTTTTTTTCTTCCATTTCAGGTGTCGTGAAAA |
| 123 | Chicken β-Actin/Rabbit β- | CGGCGGGCGGGAACGTTGCCTTCGCCCCGTGC |
| | globin chimeric 5′UTR- | CCCGCTCCGCGCCGCCTCGCGCCGCCCGCCCC |
| | intron-tbs consensus (exonic | GGCTCTGACTGACCGCGTTACTCCCACAGGTG |
| | sequence in bold (spliced | AGCGGGCGGGACGGCCCTTCTCCCTCCGGGCT |
| | together to become 5′UTR | GTAATTAGCGCTTGGTTTAATGACGGCTCGTTTC |
| | leader), tbs consensus in | TTTTCTGTGGCTGCGTGAAAGCCTTAAAGGGCT |
| | italics) | CCGGGAGGGCCTTTGTGCGGGGGGGAGCGGCT |
| | | CGGGGGGTGCGTGCGTGTGTGTGTGCGTGGGG |
| | | AGCGCCGCGTGCGGCCCGCGCTGCCCGGCGG |
| | | CTGTGAGCGCTGCGGGCGCGGCGCGGGGCTTT |
| | | GTGCGCTCCGCGTGTGCGCGAGGGGAGCGCG |
| | | GGCCGGGGGCGGTGCCCCGCGGTGCGGGGGG |
| | | GCTGCGAGGGGAACAAAGGCTGCGTGCGGGGT |
| | | GTGTGCGTGGGGGGGTGAGCAGGGGGTGTGG |
| | | GCGCGGCGGTCGGGCTGTAACCCCCCCCTGGC |
| | | ACCCCCCTCCCCGAGTTGCTGAGCACGGCCCG |
| | | GCTTCGGGTGCGGGGCTCCGTGCGGGGCGTGG |
| | | CGCGGGGCTCGCCGTGCCGGGCGGGGGGGG |
| | | CGGCAGGTGGGGGTGCCGGGGGGGGGGGGGC |
| | | CGCCTCGGGCCGGGGAGGGCTCGGGGGAGGG |
| | | GCGCGGCGGCCCCGGAGCGCCGGCGGCTGTC |
| | | GAGGCGCGGCGAGCCGCAGCCATTGCCTTTTAT |
| | | GGTAATCGTGCGAGAGGGCGCAGGGACTTCCTT |
| | | TGTCCCAAATCTGGCGGAGCCGAAATCTGGGAG |
| | | GCGCCGCCGCACCCCCTCTAGCGGGCGCGGGC |
| | | GAAGCGGTGCGGCGCCGGCAGGAAGGAAATGG |
| | | GCGGGGAGGGCCTTCGTGCGTCGCCGCGCCGC |
| | | CGTCCCCTTCTCCATCTCCAGCCTCGGGGCTGC |
| | | CGCAGGGGGACGGCTGCCTTCGGGGGGGACG |
| | | GGGCAGGGGGGGGTTCGGCTTCTGGCGTGTGA |
| | | CCGGCGGCTTTAGAGCCTCTGCTAACCATGTTC |
| | | ATGCCTTCTTCTTTTTCCTACAGCTCCTGGGCAA |
| | | A[KAGN₂₋₃]₁₀₋₁₁ |
| 124 | EF1a 5′UTR-intron-tbs | CTTTTTCGCAACGGGTTTGCCGCCAGAACACAG |
| | consensus (exonic | GTAAGTGCCGTGTGTGGTTCCCGCGGGCCTGG |
| | sequence in bold (spliced | CCTCTTTACGGGTTATGGCCCTTGCGTGCCTTG |
| | together to become 5′UTR | AATTACTTCCACCTGGCTGCAGTACGTGATTCTT |
| | leader), tbs consensus in | GATCCCGAGCTTCGGGTTGGAAGTGGGTGGGA |
| | italics) | GAGTTCGTGGCCTTGCGCTTAAGGAGCCCCTTC |
| | | GCCTCGTGCTTGAGTTGTGGCCTGGCCTGGGC |
| | | GCTGGGGCCGCCGCGTGCGAATCTGGTGGCAC |
| | | CTTCGCGCCTGTCTCGCTGCTTTCGATAAGTCTC |
| | | TAGCCATTTAAAATTTTTGATGACCTGCTGCGAC |
| | | GCTTTTTTTCTGGCAAGATAGTCTTGTAAATGCG |
| | | GGCCAAGATCAGCACACTGGTATTTCGGTTTTTG |
| | | GGGCCGCGGGCGGCGACGGGGCCCGTGCGTC |
| | | CCAGCGCACATGTTCGGCGAGGCGGGGCCTGC |
| | | GAGCGCGGCCACCGAGAATCGGACGGGGGTAG |
| | | TCTCAAGCTGCCCGGCCTGCTCTGGTGCCTGGC |
| | | CTCGCGCCGCCGTGTATCGCCCCGCCCTGGGC |
| | | GGCAAGGCTGGCCCGGTCGGCACCAGTTGCGT |
| | | GAGCGGAAAGATGGCCGCTTCCCGGCCCTGCT |
| | | GCAGGGAGCACAAAATGGAGGACGCGGCGCTC |
| | | GGGAGAGCGGGCGGGTGAGTCACCCACACAAA |
| | | GGAAAAGGGCCTTTCCGTCCTCAGCCGTCGCTT |
| | | CATGTGACTCCACGGAGTACCGGGCGCCGTCC |
| | | AGGCACCTCGATTAGTTCTCCAGCTTTTGGAGTA |
| | | CGTCGTCTTTAGGTTGGGGGGAGGGGTTTTATG |
| | | CGATGGAGTTTCCCCACACTGAGTGGGTGGAGA |
| | | CTGAAGTTAGGCCAGCTTGGCACTTGATGTAATT |
| | | CTCCTTGGAATTTGCCCTTTTTGAGTTTGGATCT |
| | | TGGTTCATTCTCAAGCCTCAGACAGTGGTTCAAA |
| | | GTTTTTTTCTTCCATTTCAGGTGTCGTGAAAA |
| | | [KAGN₂₋₃]₁₀₋₁₁ |
| 125 | TRAP binding site variant- | GAGUUUAGCGGAGUGGAGAAGAGCGGAGCCGA |
| | [KAGNN]₁₁ | GCCUAGCAGAGACGAGUGGAGCU |
| 126 | TRAP binding site variant- | GAGUUUAGCGGAGUGGAGAAGAGCGGAGCCGA |
| | [KAGNN]₁₁ | GCCUAGCAGAGACGAGAAGAGCU |
| 127 | TRAP binding site variant- | UAGUUUAGUUUAGUUUAGUUUAGUUUAGUU |
| | [KAGNN]₆ | |
| 128 | TRAP binding site variant- | UAGUUUAGUUGAGUUUAGUUGAGUUUAGUU |
| | [KAGNN]₆ | |
| 129 | TRAP binding site variant- | GAGUUUGAGUUGAGUUGAGUUUGAGUUGAGUU |
| | [KAGNN]₆ | |
| 130 | TRAP binding site variant- | UAGUUUGAGUUUAGUUGAGUUUUAGUUGAGUU |
| | [KAGNN]₆ | |
| 131 | TRAP binding site variant- | GAGTTTAGCGGAGTGGAGAAGAGCGGAGCCGA |
| | [KAGNN]₁₁ | GCCTAGCAGAGACGAGTGGAGCT |
| 132 | TRAP binding site variant- | GAGTTTAGCGGAGTGGAGAAGAGCGGAGCCGA |
| | [KAGNN]₁₀ | GCCTAGCAGAGACGAGAA |
| 133 | TRAP binding site variant- | GAGTTTAGCGGAGTGGAGAAGAGCGGAGCCGA |
| | [KAGNN]₉ | GCCTAGCAGAGAC |
| 134 | TRAP binding site variant- | GAGTTTAGCGGAGTGGAGAAGAGCGGAGCCGA |
| | [KAGNN]₈ | GCCTAGCA |
| 135 | TRAP binding site variant- | GAGTTTAGCGGAGTGGAGAAGAGCGGAGCCGA |
| | [KAGNN]₇ | GCC |
| 136 | TRAP binding site variant- | GAGTTTAGCGGAGTGGAGAAGAGCGGAGCC |
| | [KAGNN]₆ | |
| 137 | TRAP binding site variant- | GAGTTTAGCGGAGTGGAGAAAGAGACGGAGCC |
| | [KAGNNN]₃[KAGNN]₈ | GAGACCTAGCAGAGACGAGAAGAGCT |
| 138 | TRAP binding site variant- | GAGTTTAGCGGAGTGGAGAAGAGACGGAGCCG |
| | [KAGNNN]₁[KAGNN]₁₀ | AGCCTAGCAGAGACGAGAAGAGCT |
| 139 | TRAP binding site variant- | GAGTTTAGCGGAGTGGAGAAGAGCGGAGCCGA |
| | [KAGNN]₁₁ | GCCTAGCAGAGACGAGAAGAGCT |
| 140 | TRAP binding site variant- | GAGTTTAGCGGAGTGGAGAAAGAGCGGAGCCG |
| | [KAGNNN]₁[KAGNN]₇ | AGCCTAGCA |
| 141 | TRAP binding site variant- | GAGTTTAGCGGAGTGGAGAAAGAGCGGAGCCG |
| | [KAGNNN]₁[KAGNN]₆ | AGCC |
| 142 | 3′ tbs-Kozak junction variant | GAGATG |
| 143 | 3′ tbs-Kozak junction variant | KAGVATG |
| 144 | 3′ tbs-Kozak junction variant | KAGVVATG |
| 145 | 3′ tbs-Kozak junction variant | KAGRVVATG |
| 146 | 3′ tbs-Kozak junction variant | KAGNRVVATG |
| 147 | Optimal 3′ tbs-Kozak | KAGCCGAGATG |
| | junction variant | |
| 148 | Optimal 3′ tbs-Kozak | KAGNGGAGCCATG |
| | junction variant | |
| 149 | Optimal 3′ tbs-Kozak | KAGNNGAGACCATG |
| | junction variant | |
| 150 | 3′ tbs-Kozak junction variant | KAGGCGAGCATG |
| 151 | Spacer variant | ATAGCAGAGACGGCT |
| 152 | Spacer variant | ATAGCAGAGA |
| 153 | Spacer variant | ATAGC |
| 154 | Spacer variant | ATATCAGAGACGGCTAGCGTATACCA |
| 155 | Spacer variant | ATATCAGAGACGGCT |
| 156 | Spacer variant | AGAGACGGCT |
| 157 | Spacer variant | TACCA |
| 158 | 3′ tbs-MCS-Kozak variant | GAGCTCTAGAVVATG |
| 159 | 3′ tbs-MCS-Kozak variant | GAGCTCGTCGACVATG |
| 160 | 3′ tbs-MCS-Kozak variant | GAGCTCGAATTCGAAVVATG |
| 161 | 3′ tbs-MCS-Kozak variant | GAGCTCTAGACGTCGACVATG |
| 162 | 3′ tbs-MCS-Kozak variant | GAGCTCTAGAATTCGAAVVATG |
| 163 | 3′ tbs-MCS-Kozak variant | GAGCTCTAGATATCGATRVVATG |
| 164 | 3′ tbs-MCS-Kozak variant | KAGACTAGTACTTAAGCTTRVVATG |
| 165 | 3′ tbs-MCS-Kozak variant | GAGCTCTAGACCATG |
| 166 | 3′ tbs-MCS-Kozak variant | GAGCTCGTCGACCATG |
| 167 | 3′ tbs-MCS-Kozak variant | GAGCTCGAATTCGAACCATG |
| 168 | 3′ tbs-MCS-Kozak variant | GAGCTCTAGACGTCGACCATG |
| 169 | 3′ tbs-MCS-Kozak variant | GAGCTCTAGAATTCGAACCATG |
| 170 | 3′ tbs-MCS-Kozak variant | GAGCTCTAGATATCGATACCATG |
| 171 | 3′ tbs-MCS-Kozak variant | KAGACTAGTACTTAAGCTTACCATG |
| 172 | Illustrative nucleic acid | CTTTTTCGCAACGGGTTTGCCGCCAGAACACAG |
| | sequence 1 containing L33 | GAGTTTAGCGGAGTGGAGAAGAGCGGAGCCGA |
| | Improved leader, optimal | GCCGAGATG |
| | (overlapping) tbs | |
| | ([KAGNN]₈)-Kozak junction | |
| 173 | Illustrative nucleic acid | CTTTTTCGCAACGGGTTTGCCGCCAGAACACAG |
| | sequence 2 containing L33 | GAGTTTAGCGGAGTGGAGAAGAGCGGAGCCGA |
| | Improved leader, optimal | GCCTAGCAGAGACGAGCCGAGATG |
| | (overlapping) tbs | |
| | ([KAGNN]₁₁)-Kozak junction | |
| 174 | Illustrative nucleic acid | CTTTTTCGCAACGAGTTTAGCGGAGTGGAGAAG |
| | sequence 3 containing L12 | AGCGGAGCCGAGCCTAGCAGAGACGAGCCGAG |
| | Improved leader, optimal | ATG |
| | (overlapping) tbs | |
| | ([KAGNN]₁₁)-Kozak junction | |
| 175 | Illustrative nucleic acid | CTTTTTCGCAACGGGTTTGCCGCCAGAACACAG |
| | sequence 4 for intron- | GTGTCGTGAAAAGAGTTTAGCGGAGTGGAGAAG |
| | containing 5′UTRs, resulting | AGCGGAGCCGAGCCTAGCAGAGACGAGCCGAG |
| | in a spliced leader | ATG |
| | comprising L33, optimal | |
| | (overlapping) tbs | |
| | ([KAGNN]₁₁)-Kozak junction | |
| 176 | Illustrative nucleic acid | ATAGCAGAGACGGCTGAGTTTAGCGGAGTGGAG |
| | sequence 5 containing | AAGAGCGGAGCCGAGCCGAGATG |
| | improved spacer, optimal | |
| | (overlapping) tbs | |
| | ([KAGNN]₈)-Kozak junction | |
| 177 | Illustrative nucleic acid | CTTTTTCGCAACGGGTTTGCCGCCAGAACACAG |
| | sequence 6 containing L33 | GAGTTTAGCGGAGTGGAGAAGAGCGGAGCCGA |
| | Improved leader tbs | GCCTAGCAGAGACGAGAAGAGCTCTAGACCATG |
| | ([KAGNN]₁₁)-MCS-Kozak | |
| 178 | Illustrative nucleic acid | ATAGCAGAGACGGCTGAGTTTAGCGGAGTGGAG |
| | sequence 7 containing | AAGAGCGGAGCCGAGCCTAGCAGAGACGAGAA |
| | improved spacer, tbs | GAGCTCTAGACCATG |
| | ([KAGNN]₁₁)-MCS-Kozak | |

Tryptophan RNA-binding attenuation protein (TRAP) is a bacterial protein that has been extensively characterised in Bacillus subtilis. TRAP is described in WO 2015/092440.

The TRAP open-reading frame may be codon-optimised for expression in mammalian (e.g. Homo sapiens) cells, since the bacterial gene sequence is likely to be non-optimal for expression in mammalian cells. The sequence may also be optimised by removing potential unstable sequences and splicing sites. The use of a HIS-tag C-terminally expressed on the TRAP protein appears to offer a benefit in terms of translation repression and may optionally be used. This C-terminal HIS-tag may improve solubility or stability of the TRAP within eukaryotic cells, although an improved functional benefit cannot be excluded. Nevertheless, both HIS-tagged and untagged TRAP allowed robust repression of transgene expression. Certain cis-acting sequences within the TRAP transcription unit may also be optimised; for example, EF1a promoter-driven constructs enable better repression with low inputs of TRAP plasmid compared to CMV promoter-driven constructs in the context of transient transfection.

In one embodiment, the TRAP for use in the present invention is derived from a bacteria.

In one embodiment of the present invention, TRAP is derived from a Bacillus species, for example Bacillus subtilis. For example, TRAP may comprise the sequence as set forth in SEQ ID NO: 94.

In a preferred embodiment of the present invention, SEQ ID NO: 94 is C-terminally tagged with six histidine amino acids (HIS×6 tag).

In an alternative embodiment, TRAP is derived from Aminomonas paucivorans. For example, TRAP may comprise the sequence as set forth in SEQ ID NO: 95.

In an alternative embodiment, TRAP is derived from Desulfotomaculum hydrothermale. For example, TRAP may comprise the sequence as set forth in SEQ ID NO: 96.

In an alternative embodiment, TRAP is derived from B. stearothermophilus. For example, TRAP may comprise the sequence as set forth in SEQ ID NO: 97.

In an alternative embodiment, TRAP is derived from B. stearothermophilus S72N. For example, TRAP may comprise the sequence as set forth in SEQ ID NO: 98.

In an alternative embodiment, TRAP is derived from B. halodurans. For example, TRAP may comprise the sequence as set forth in SEQ ID NO: 99.

In an alternative embodiment, TRAP is derived from Carboxydothermus hydrogenoformans. For example, TRAP may comprise the sequence as set forth in SEQ ID NO: 100.

In one embodiment, TRAP is encoded by the tryptophan RNA-binding attenuation protein gene family mtrB (TrpBP superfamily e.g. with NCBI conserved domain database #c103437).

In preferred embodiments, the TRAP is C-terminally tagged with six histidine amino acids (HIS×6 tag).

In a preferred embodiment, TRAP comprises an amino acid sequence that has 50%, 60%, 70%, 80%, 90%, 95%, 99% or 100% identity to any of SEQ ID NOs: 94 to 100 and is capable of interacting with an RNA-binding site such that expression of an operably linked NOI is modified, for example repressed or prevented, in a viral vector production cell.

In a preferred embodiment, TRAP comprises an amino acid sequence that has at least 50%, 55%, 60%, 65%, 70%, 75%, 80%, 85%, 90%, 95%, 97%, 99% or 100% identity to any of SEQ ID NOs: 94 to 100 and is capable of interacting with an RNA-binding site such that expression of an operably linked NOI is modified, for example repressed or prevented, in a viral vector production cell.

In another embodiment, TRAP may be encoded by a polynucleotide comprising a nucleotide sequence which encodes a protein which is capable of interacting with an RNA-binding site such that expression of an operably linked NOI is modified, for example repressed or prevented, in a viral vector production cell. For example, TRAP may be encoded by a polynucleotide comprising a nucleotide sequence which encodes a protein of SEQ ID NOs: 94 to 100.

All variants, fragments or homologues of TRAP for use in the invention will retain the ability to bind to the TRAP binding site as described herein such that translation of the NOI (which may be a marker gene) is repressed or prevented in a viral vector production cell.

TRAP Binding Site

The term “binding site” is to be understood as a nucleic acid sequence that is capable of interacting with a certain protein.

A consensus TRAP binding site sequence that is capable of binding TRAP is [KAGNN] repeated multiple times (e.g. 6, 7, 8, 9, 10, 11, 12 or more times); such sequence is found in the native trp operon. In the native context, occasionally AAGNN is tolerated and occasionally additional “spacing” N nucleotides result in a functional sequence. In vitro experiments have demonstrated that at least 6 or more consensus repeats are required for TRAP-RNA binding (Babitzke P, Y. J., Campanelli D. (1996) Journal of Bacteriology 178(17): 5159-5163). Therefore, preferably in one embodiment there are 6 or more continuous [KAGN_{≥2}] sequences present within the tbs, wherein K may be T or G in DNA and U or G in RNA.

For TRAP as the RNA-binding protein, preferably the TRIP system works maximally with a tbs sequence containing at least 8 KAGNN repeats, although 7 repeats may be used to still obtain robust transgene repression, and 6 repeats may be used to allow sufficient repression of the transgene to levels that could rescue vector titres. Whilst the KAGNN consensus sequence may be varied to maintain TRAP-mediated repression, preferably the precise sequence chosen may be optimised to ensure high levels of translation in the non-repressed state. For example, the tbs sequences may be optimised by removing splicing sites, unstable sequences or stem-loops that might hamper translation efficiency of the mRNA in the absence of TRAP (i.e. in target cells). Regarding the configuration of the KAGNN repeats of a given tbs, the number of N “spacing” nucleotides between the KAG repeats is preferably two. However, a tbs containing more than two N spacers between at least two KAG repeats may be tolerated (as many as 50% of the repeats containing three Ns may result in a functional tbs as judged by in vitro binding studies; Babitzke P, Y. J., Campanelli D. (1996) Journal of Bacteriology 178(17): 5159-5163). Indeed, it has been shown that an 11×KAGNN tbs sequence can tolerate up to three replacements with KAGNNN repeats and still retain some potentially useful translation-blocking activity in partnership with TRAP-binding.

In one embodiment of the present invention, the TRAP binding site or portion thereof comprises the sequence KAGN_{≥2} (e.g. KAGN₂₋₃). For the avoidance of doubt therefore, this tbs or portion thereof comprises, for example, any of the following repeat sequences: UAGNN, GAGNN, TAGNN, UAGNNN, GAGNNN, or TAGNNN.

“N” is to be understood as specifying any nucleotide at that position in the sequence. For example, this could be G, A, T, C or U. The number of such nucleotides is preferably 2 but up to three, for example 1, 2 or 3, KAG repeats of an 11× repeat tbs or portion thereof may be separated by 3 spacing nucleotides and still retain some TRAP-binding activity that leads to translation repression. Preferably not more than one N₃ spacer will be used in an 11× repeat tbs or portion thereof in order to retain maximal TRAP-binding activity that leads to translation repression.

In another embodiment, the tbs or portion thereof comprises multiple repeats of KAGN_{≥2} (e.g. multiple repeats of KAGN₂₋₃).

In another embodiment, the tbs or portion thereof comprises multiple repeats of the sequence KAGN₂.

In another embodiment, the tbs or portion thereof comprises at least 6 repeats of KAGN_{≥2} (e.g. at least 6 repeats of KAGN₂₋₃).

In another embodiment, the tbs or portion thereof comprises at least 6 repeats of KAGN_{≥2}. For example, the tbs or portion thereof may comprise 6, 7, 8, 9, 10, 11, 12 or more repeats of KAGN_{≥2}. For example, the tbs or portion thereof may comprise any one of SEQ ID NOs: 125-136 or 139.

In another embodiment, the tbs or portion thereof comprises at least 8 repeats of KAGN_{≥2} (e.g. at least 8 repeats of KAGN₂₋₃). For example, the tbs or portion thereof may comprise any one of SEQ ID NOs: 125, 126, 131-134, 137-24.

Preferably, the number of KAGNNN repeats present in the tbs or portion thereof is 1 or less. For example, the tbs or portion thereof may comprise any one of SEQ ID NOs: 125, 126, 131-134, 136-141.

In another embodiment, the tbs or portion thereof comprises 11 repeats of KAGN_{≥2} (e.g. 11 repeats of KAGN₂₋₃). Preferably, the number of KAGNNN repeats present in this tbs or portion thereof is 3 or less. For example, the tbs or portion thereof may comprise any one of SEQ ID NOs: 125, 126, 131, 137-139.

In another embodiment, the tbs or portion thereof comprises 12 repeats of KAGN_{≥2} (e.g. 12 repeats of KAGN₂₋₃).

In a preferred embodiment, the tbs or portion thereof comprises 8-11 repeats of KAGN₂ (e.g. 8, 9, 10 or 11 repeats of KAGN₂). For example, the tbs or portion thereof may comprise any one of SEQ ID NOs: 125, 126, 131-134, 137-141.

In one embodiment, the TRAP binding site or portion thereof may comprise any of SEQ ID NOs: 125-141.

For example, the TRAP binding site or portion thereof may comprise a sequence as set forth in SEQ ID NO: 125 or SEQ ID NO: 126.

By “repeats of KAGN_{≥2}” it is to be understood that the general KAGN_{≥2} (e.g. KAGN₂₋₃) motif is repeated. Different KAGN_{≥2} sequences satisfying the criteria of this motif may be joined to make up the tbs or portion thereof. It is not intended that the resulting tbs or portion thereof is limited to repeats of only one sequence that satisfies the requirements of this motif, although this possibility is included in the definition. For example, “6 repeats of KAGN_{≥2}” includes, but is not limited to, a sequence as set forth in SEQ ID NOs: 127-130.

An 8-repeat tbs or portion thereof containing one KAGNNN repeat and seven KAGNN repeats retains TRAP-mediated repression activity. Less than 8-repeat tbs sequences or portions thereof (e.g. 7- or 6-repeat tbs sequences or portions thereof) containing one or more KAGNNN repeats may have lower TRAP-mediated repression activity. Accordingly, when fewer than 8-repeats are present, it is preferred that the tbs or portion thereof comprises only KAGNN repeats.

Preferred nucleotides for use in the KAGNN repeat consensus are a pyrimidine in at least one of the NN spacer positions; a pyrimidine at the first of the NN spacer positions; pyrimidines at both of the NN spacer positions; G at the K position.

It is also preferred that G is used at the K position when the NN spacer positions are AA (i.e. it is preferred that TAGAA is not used as a repeat in the consensus sequence).

By “capable of interacting” it is to be understood that the nucleic acid binding site (e.g. tbs or portion thereof) is capable of binding to a protein, for example TRAP, under the conditions that are encountered in a cell, for example a eukaryotic viral vector production cell. Such an interaction with an RNA-binding protein such as TRAP results in the repression or prevention of translation of a NOI to which the nucleic acid binding site (e.g. the tbs or portion thereof) is operably linked.

By “operably linked” it is to be understood that the components described are in a relationship permitting them to function in their intended manner. Therefore a tbs or portion thereof for use in the invention operably linked to a NOI is positioned in such a way that translation of the NOI is modified when as TRAP binds to the tbs or portion thereof.

Placement of a tbs or portion thereof capable of interacting with TRAP upstream of a NOI translation initiation codon of a given open reading frame (ORF) allows specific translation repression of mRNA derived from that ORF. The number of nucleotides separating the tbs or portion thereof and the translation initiation codon may be varied, for example from 0 to 34 nucleotides, without affecting the degree of repression. As a further example, 0 to 13 nucleotides may be used to separate the TRAP-binding site or portion thereof and the translation initiation codon.

The tbs or portion thereof may be placed downstream of an internal ribosome entry site (IRES) to repress translation of the NOI in a multicistronic mRNA. Indeed, this supplies further evidence that tbs-bound TRAP might block the passage of the 40S ribosome; IRES elements function to sequester the 40S ribosome subunit to an mRNA in a CAP-independent manner before the full translation complex is formed (see Thompson, S. (2012) Trends in Microbiology 20(11): 558-566) for a review on IRES translation initiation). Thus, it is possible for the TRIP system to repress multiple open-reading frames from a single mRNA expressed from viral vector genomes. This will be a useful feature of the TRIP system when producing vectors encoding multiple therapeutic genes, especially when all the transgene products might negatively affect vector titres to some degree.

In one embodiment, the nucleotide sequence comprises a spacer sequence between an IRES and the tbs or the portion thereof. The IRES may be an IRES as described herein under the subheading “Internal ribosome entry site”. The spacer sequence may be between 0 and 30 nucleotides in length, preferably 15 nucleotides in length. The spacer may comprise the sequence as defined in any one of SEQ ID NOs:151-157, preferably the spacer comprises a sequence as defined in SEQ ID NO:151.

In one embodiment, the spacer sequence between an IRES and the tbs or portion thereof is 3 or 9 nucleotides from the 3′ end of the tbs or portion thereof and the downstream initiation codon of the NOI.

In one embodiment, the tbs or portion thereof lacks a type II restriction enzyme site. In a preferred embodiment, the tbs or portion thereof lacks a SapI restriction enzyme site.

In some embodiments, the nucleotide sequence further comprises an RRE sequence or functional substitute thereof.

Overlapping Kozak Sequence and TRAP Binding Site Sequences

The present inventors have surprisingly found that improved levels of repression can be achieved by ‘hiding’ the Kozak sequence within the 3′ terminus of the tbs or portion thereof (using overlapping tbs and Kozak sequences; see FIGS. 19B and 19C), compared to the use of non-overlapping tbs and Kozak sequences. In addition, all of the tested overlapping Kozak and tbs sequences unexpectedly directed efficient levels of translation initiation, i.e. the tested overlapping sequences provided similar levels of transgene expression to the non-overlapping Kozak and tbs sequences in the absence of TRAP. Without wishing to be bound by theory, the improved levels of repression can be attributed to improved occlusion of the transgene initiation codon by the TRAP-tbs complex when the tbs or potion thereof overlaps the Kozak sequence.

The term “Kozak sequence” is to be understood as a consensus sequence in eukaryotic mRNA which is recognised by the ribosome as the translational start site. The Kozak sequence includes the ATG initiation (start) codon in DNA (AUG in mRNA). The exact Kozak sequence present in eukaryotic mRNA determines the efficiency of translation initiation, i.e. certain Kozak sequences will not lead to efficient translation initiation.

The full Kozak sequence is typically understood to have the consensus sequence (gcc)gccRccATGG for DNA and (gcc)gccRccAUGG for RNA, wherein: a lowercase letter denotes the most common base at a position where the base at this position can vary; an uppercase letter denotes a highly conserved base at this position; “R” denotes that a purine (i.e. A or G) is typically optimal at this position; and the sequence in parentheses (gcc) is of uncertain significance. T/U is generally the least preferred nucleotide at all of the positions of the Kozak sequence consensus that are upstream of the initiation codon.

As the first three bases of the full Kozak sequence are of uncertain significance, the Kozak sequence can also be understood to have the consensus sequence, referred to herein as the “extended Kozak sequence”, GNNRVVATGG for DNA (SEQ ID NO: 103) and GNNRVVAUGG for RNA, wherein “R” is to be understood as specifying a purine (i.e. A or G) at that position in the sequence, “V” is to be understood as specifying any nucleotide from G, A, or C and “N” is to be understood as specifying any nucleotide at that position in the sequence. For example, “N” could be G, A, T, C or U. It should be noted that the “R” at position −1 and “G” and position +3 (relative to the “A” of the ATG being position 0) are considered the most important positions in terms of Kozak strength. However, the presence of the “G” at position +3 in transgene sequences is dependent on the ORF being encoded, and so for the purposes of the specification the +3 position has not been considered as part of the ‘core’ Kozak sequence.

The bases found at the first six positions of the full Kozak sequence vary such that any base can be found at those positons (denoted (gcc)gcc above). Thus, the full Kozak consensus sequence can be considered to contain a ‘core’ Kozak sequence which consists of the portion of the full Kozak sequence having reduced variability, denoted RccAUG above. The ‘core’ Kozak consensus sequence is defined herein as: RVVAUG for mRNA and RVVATG for DNA, wherein “R” is to be understood as specifying a purine (i.e. A or G) at that position in the sequence and “V” is to be understood as specifying any nucleotide from G, A, or C.

In one preferred embodiment of the present invention, the Kozak sequence comprises the sequence RVVATG (SEQ ID NO: 104); wherein “R” is to be understood as specifying a purine (i.e. A or G) at that position in the sequence and “V” is to be understood as specifying any nucleotide from G, A, or C.

In one embodiment of the present invention, the Kozak sequence comprises the sequence RNNATG; wherein “R” is to be understood as specifying a purine (i.e. A or G) at that position in the sequence and “N” is to be understood as specifying any nucleotide from G, A, T/U or C, recognising that use of a “T/U” may give rise to reduced levels of expression in the absence of TRAP.

In some embodiments, the Kozak sequence overlaps the 3′ terminal KAGNN repeat of the TRAP binding site or of the portion thereof. Thus, the core Kozak sequence may overlap the the 3′ terminal KAGNN repeat of the TRAP binding site or of the portion thereof.

A summary of preferred overlapping tbs and Kozak consensus sequences is provided in FIG. 19C.

In a preferred embodiment, the 3′ terminal KAGNN repeat of the TRAP binding site or of the portion thereof overlaps at least the first, or first two, nucleotides of the ATG triplet within the core Kozak sequence.

As described herein, In one aspect of the present invention, the 3′ terminal KAGNN repeat of the TRAP binding site or of the portion thereof overlaps the ATG start codon of the nucleotide of interest (transgene ORF). In one aspect the 3′ terminal KAGNN repeat of the TRAP binding site or of the portion thereof overlaps the first one, or two, nucleotides of the ATG start codon of the nucleotide of interest (transgene ORF).

In one aspect, the overlapping tbs-Kozak sequence may have the consensus sequence KAGNNG (SEQ ID NO: 113), wherein “NN” is the first two nucleotides within the ATG triplet of the Kozak sequence.

The consensus sequence may be KAGATG (SEQ ID NO: 114); wherein “K” is either G or T/U.

In one aspect, the overlapping tbs-Kozak sequence may be GAGATG (SEQ ID NO: 142) as shown in FIG. 19C.

In one embodiment, the 3′ terminal KAGNN repeat of the TRAP binding site or of the portion thereof overlaps the first nucleotide of the ATG triplet within the nucleotide of interest.

In one aspect the sequence may comprise the sequence KAGNNTG (SEQ ID NO: 115), wherein the second “N” is the first nucleotide within the ATG triplet. The consensus sequence is KAGNATG (SEQ ID NO: 116); wherein “K” is to be understood as specifying a G or T/U at that position in the sequence, “N” is to be understood as specifying any nucleotide from G, A, T, U or C, but preferably “V” i.e. from G, A or C. For example, the overlapping sequence may be KAGVATG (SEQ ID NO: 143) as shown in FIG. 19C.

In one embodiment, the overlapping Kozak sequence and TRAP binding site or portion thereof for use in the invention comprises a sequence as set forth in SEQ ID NOs: 142-146.

In one embodiment, the nucleotide sequence of the invention comprises a sequence as set forth in SEQ ID NOs: 142-146. In one aspect the sequence of the invention comprises the sequence KAGATG.

Preferred overlapping tbs or portion thereof and core Kozak sequences corresponding to the consensus sequences GAGATG (SEQ ID NO: 142), KAGVATG (SEQ ID NO: 143) and KAGVVATG (SEQ ID NO: 144) for use in the nucleic acid of the invention (based upon the consensus tbs repeat sequence of KAGNN as defined herein and the consensus ‘core’ Kozak sequence RVVATG as defined herein) encompass sequences as set forth in SEQ ID NOs: 142 and 69-92.

In some embodiments, the nucleotide sequence comprises a sequence as set forth in SEQ ID NOs: 147-150. Preferably, the nucleotide sequence comprises a sequence as set forth in SEQ ID NO: 147 or SEQ ID NO: 148.

In a preferred embodiment, the nucleotide sequence of the invention comprises the overlapping tbs and Kozak sequence set forth in SEQ ID NO: 106.

To improve the tractability of the TRIP system it is desirable to be able to have the ability to clone a NOI directly into the expression cassette containing the promoter-5′UTR-tbs sequence via the option of several different restriction enzymes (RE), i.e. to incorporate a multiple cloning site (MCS) between the tbs and the Kozak sequence (see FIG. 19A). The present inventors have demonstrated that several different MCS can be tolerated by the TRIP system, i.e. transgene repression is still obtained when an MCS is used. This was unexpected given that 5′UTR leader sequences can modulate the degree of TRAP-mediated repression, that the close proximity of the tbs to the ATG initiation codon is important, and that an efficient Kozak sequence must be maintained with or between the MCS and the initiation codon of the NOI to ensure efficient translation initiation. In addition, the number and/or combinations of RE sites that could be used whilst maintaining TRAP-mediated repression could not be predicted.

As such, sequence ‘compression’ was required such that several (overlapping) RE sites could be incorporated in as short a distance as possible from the tbs to the ATG initiation codon (to retain proximity of tbs to ATG), whilst also maintaining an efficient core Kozak sequence of RVVATG.

In one embodiment, the nucleotide sequence further comprises a tbs or a portion thereof as described herein, a multiple cloning site (MCS) and a Kozak sequence as described herein, wherein said MCS is located downstream of the tbs or portion thereof and upstream of the Kozak sequence. Suitably, the tbs or portion thereof and the Kozak sequence do not overlap.

As used herein, a “multiple cloning site” is to be understood as a DNA region which contains several restriction enzyme recognition sites (restriction enzyme sites) very close to each other. In one embodiment, the RE sites may be overlapping in the MCS for use in the invention.

As used herein, a “restriction enzyme site” or “restriction enzyme recognition site” is a location on a DNA molecule containing specific sequences of nucleotides, 4-8 nucleotides in length, which are recognised by restriction enzymes. A restriction enzyme recognises a specific RE site (i.e. a specific sequence) and cleaves the DNA molecule within, or nearby, the RE site.

In one embodiment, the nucleotide sequence comprises a sequence as set forth in SEQ ID NOs: 158-171.

In one embodiment, the nucleotide sequence comprises a sequence as set forth in SEQ ID NOs: 165-171.

In one embodiment, the nucleotide sequence comprises a sequence as set forth in SEQ ID NOs 165, 168 or 171.

In a preferred embodiment, the nucleotide sequence of the invention comprises the overlapping tbs-MCS-Kozak sequence as set forth in SEQ ID NO: 107.

In one embodiment, the Kozak sequence comprises the sequence RNNATG; wherein “R” is to be understood as specifying a purine (i.e. A or G) at that position in the sequence, and “N” is to be understood as specifying any nucleotide from G, A, T/U or C.

Repression or prevention of the translation of the NOI is to be understood as alteration of the amount of the product (e.g. protein) of the NOI that is translated during viral vector production in comparison to the amount expressed in the absence of the nucleotide sequence of the invention at the equivalent time point. Such alteration of translation results in a consequential repression or prevention of the expression of the protein encoded by the NOI.

In one embodiment, the nucleotide sequence of the invention is capable of interacting TRAP, such that translation of the nucleotide of interest is repressed or prevented in a viral vector production cell.

The translation of the NOI at any given time during vector production may be reduced to 90%, 80%, 70%, 60%, 50%, 40%, 30%, 20%, 10%, 5%, 4%, 3%, 2%, 1%, 0.5% or 0.1% of the amount translated in the absence of the nucleotide sequence of the invention at the same time-point in vector production.

The translation of the NOI at any given time during vector production may be reduced to less than 90%, 80%, 70%, 60%, 50%, 40%, 30%, 20%, 10%, 5%, 4%, 3%, 2%, 1%, 0.5% or 0.1% of the amount translated in the absence of the nucleotide sequence of the invention at the same time-point in vector production.

In the context of the present invention, the translation of the NOI at any given time during vector production may be reduced to 90%, 80%, 70%, 60%, 50%, 40%, 30%, 20%, 10%, 5%, 4%, 3%, 2%, 1%, 0.5% or 0.1% of the amount translated in the presence of a nucleic acid sequence comprising non-overlapping Kozak and tbs sequences (as opposed to the nucleotide sequence of the invention) at the same time-point in vector production.

In the context of the present invention the translation of the NOI at any given time during vector production may be reduced to less than 90%, 80%, 70%, 60%, 50%, 40%, 30%, 20%, 10%, 5%, 4%, 3%, 2%, 1%, 0.5% or 0.1% of the amount translated in the presence of a nucleic acid sequence comprising non-overlapping Kozak and tbs sequences (as opposed to the nucleotide sequence of the invention) at the same time-point in vector production.

Preventing the translation of the NOI is to be understood as reducing the amount of translation to substantially zero.

The expression of the protein from the NOI at any given time during vector production may be reduced to 90%, 80%, 70%, 60%, 50%, 40%, 30%, 20%, 10%, 5%, 4%, 3%, 2%, 1%, 0.5% or 0.1% of the amount expressed in the absence of the nucleotide sequence of the invention at the same time-point in vector production.

The expression of the protein from the NOI at any given time during vector production may be reduced to less than 90%, 80%, 70%, 60%, 50%, 40%, 30%, 20%, 10%, 5%, 4%, 3%, 2%, 1%, 0.5% or 0.1% of the amount expressed in the absence of the nucleotide sequence of the invention at the same time-point in vector production.

In the context of the present invention the expression of the protein from the NOI at any given time during vector production may be reduced to 90%, 80%, 70%, 60%, 50%, 40%, 30%, 20%, 10%, 5%, 4%, 3%, 2%, 1%, 0.5% or 0.1% of the amount expressed in the presence of a nucleic acid sequence comprising non-overlapping Kozak and tbs sequences (as opposed to the nucleotide sequence of the invention) at the same time-point in vector production.

In the context of the present invention the expression of the protein from the NOI at any given time during vector production may be reduced to less than 90%, 80%, 70%, 60%, 50%, 40%, 30%, 20%, 10%, 5%, 4%, 3%, 2%, 1%, 0.5% or 0.1% of the amount expressed in the presence of a nucleic acid sequence comprising non-overlapping Kozak and tbs sequences (as opposed to the nucleotide sequence of the invention) at the same time-point in vector production.

Preventing the expression of the protein from the NOI is to be understood as reducing the amount of the protein that is expressed to substantially zero.

Methods for the analysis and/or quantification of the translation of an NOI are well known in the art.

A protein product from lysed cells may be analysed using methods such as SDS-PAGE analysis with visualisation by Coomassie or silver staining. Alternatively a protein product may be analysed using Western blotting or enzyme-linked immunosorbent assays (ELISA) with antibody probes which bind the protein product. A protein product in intact cells may be analysed by immunofluorescence.

In one embodiment, the distance between the transcription start site/end of promoter to start of the tbs or of the portion thereof is less than 34 nucleotides.

In one embodiment, the distance between the transcription start site/end of promoter to start of the tbs or of the portion thereof is less than 13 nucleotides.

In one embodiment, the nucleotide sequence is a vector transgene expression cassette.

In one embodiment, the nucleic acid sequence of the invention further comprises a promoter. Typically, transcription of the promoter results in a 5′ UTR encoded in the resulting mRNA transcript. The promoter may be any promoter which is known in the art and is suitable for controlling the expression of the nucleotide of interest. For example, the promoter may be EF1a, EFS, CMV or CAG.

In a preferred embodiment, the overlapping tbs and Kozak sequence as described herein is located within the 5′ UTR of the promoter, wherein the 5′ UTR may comprise native sequence from the associated promoter, or more preferably, the 5′ UTR is composed of 5′ UTR sequences described herein.

In a preferred embodiment, the sequence comprising a compressed/overlapping MCS between the tbs and the Kozak sequence as described herein is located within said 5′ UTR.

The overlapping tbs and Kozak sequence as described herein or the sequence comprising a compressed/overlapping MCS between the tbs and the Kozak sequence as described herein may be located at the 3′ end of said 5′ UTR.

Preferably, the 5′ UTR comprises one of the following sequences: SEQ ID NO: 29-37, 45-58, 69-92 and 108-116. More preferably, the 5′ UTR comprises SEQ ID NO: 29 or SEQ ID NO: 108. Even more preferably, the 5′ UTR comprises SEQ ID NO: 29.

The promoter-5′ UTR region may comprise an intron. The intron may be a native intron or a heterologous intron. For example, the promoter may be EF1a or CAG.

The promoter may be a promoter which is typically used in viral vector genomes without an intron, for example CMV.

In a preferred embodiment, the promoter-5′ UTR region has been engineered to comprise an artificial 5′ UTR comprising a heterologous intron. Thus, the promoter-5′ UTR region has been engineered to contain a heterologous exon-intron-exon sequence, wherein the mature 5′ UTR encoded within the mRNA transcript results from splicing-out of the intron. The promoter-5′ UTR sequence may be engineered using methods known in the art. For example, the promoter may be engineered as described herein.

Preferably, expression of the transgene protein from its mature mRNA—resulting from splicing-out of the intron or heterologous intron—is efficiently repressed by TRAP. Suitably, the intron or heterologous intron may be the EF1a intron sequence as per SEQ ID No:122.

The intron or heterologous intron may be located upstream, i.e. 5′, of the overlapping tbs and Kozak sequence as described herein or of the sequence comprising an MCS between the tbs and the Kozak sequence as described herein.

The 5′ UTR may comprise the following sequence (chicken β-Actin/Rabbit β-globin chimeric 5′UTR-intron, exonic sequence in bold (spliced together to become 5′UTR leader)):

The 5′ UTR may comprise a sequence as set forth in SEQ ID NO: 121.

The 5′ UTR may comprise a sequence as set forth in SEQ ID NO: 122.

In one embodiment, the promoter comprises a sequence as set forth in SEQ ID NO: 123.

In one embodiment, the promoter comprises a sequence as set forth in SEQ ID NO: 124.

In one embodiment, the promoter comprises a sequence as set forth in SEQ ID NO: 117.

In one embodiment, the promoter comprises a sequence as set forth in SEQ ID NO: 118.

The spliced sequence corresponding to SEQ ID NO: 117 is set forth in SEQ ID NO: 119.

The spliced sequence corresponding to SEQ ID NO: 118 is set forth in SEQ ID NO: 120.

Improved Leader Sequence

In application of the TRIP system to different promoters (containing different native 5′UTRs of different lengths and composition) it is desirable to be able to be able to simply apply the tbs sequence within a promoter-UTR context to afford efficient repression by TRAP, whilst also maintaining good levels of expression without TRAP. From the outset of this work, it was not known what would be the achievable level of repression mediated by TRAP-tbs, when the tbs is inserted into native UTRs of a variety of constitutive promoters. Ideally, it would be advantageous to be able to supply a single conserved 5′UTR leader sequence together with the tbs when modifying the promoter of choice in order to avoid any of the potential variability in repression levels that might be directed by native 5′UTR sequences. Surprisingly, it was found that the first exon of the EF1a promoter (SEQ ID NO: 101) provides consistently good levels of transgene repression by TRAP compared to 5′UTR leaders comprising native leader sequences, and this leader also provides good levels of transgene expression in the absence of TRAP.

In some embodiments, the nucleotide sequence comprises a 5′ leader sequence upstream of the tbs or the portion thereof. The leader sequence may be immediately upstream of the TRAP binding site or the portion thereof, i.e. there may be no further sequences separating the leader sequence and the TRAP binding site or portion thereof. If the 5′ leader is derived from a splicing event, then the sequences from the exon/exon junction to the tbs should be kept to a minimal length (preferably 12nt). The leader sequence may comprise a sequence derived from the non-coding EF1α exon 1 region. In a preferred embodiment, the leader sequence comprises a sequence as defined in SEQ ID NO:101, SEQ ID NO:102 or SEQ ID NO: 93.

Illustrative Nucleotide Sequences

Illustrative nucleotide sequences of the invention are shown below. SEQ ID NO: 172—Illustrative nucleotide sequence 1 containing L33 Improved leader, optimal (overlapping) tbs ([KAGNN]₈)-Kozak junction

CTTTTTCGCAACGGGTTTGCCGCCAGAACACAGGAGTTTAGCGGAG
TGGAGAAGAGCGGAGCCGAGCCGAGATG

SEQ ID NO: 173—Illustrative nucleotide sequence 2 containing L33 Improved leader, optimal (overlapping) tbs ([KAGNN]₁₁)-Kozak junction

CTTTTTCGCAACGGGTTTGCCGCCAGAACACAGGAGTTTAGCGGAG
TGGAGAAGAGCGGAGCCGAGCCTAGCAGAGACGAGCCGAGATG

SEQ ID NO: 174—Illustrative nucleotide sequence 3 containing L12 Improved leader, optimal (overlapping) tbs ([KAGNN]₁₁)-Kozak

CTTTTTCGCAACGAGTTTAGCGGAGTGGAGAAGAGCGGAGCCGAGC
CTAGCAGAGACGAGCCGAGATG

SEQ ID NO: 175—Illustrative nucleotide sequence 4 for intron-containing 5′UTRs, resulting in a spliced leader comprising L33, optimal (overlapping) tbs ([KAGNN]₁₁)-Kozak junction

CTTTTTCGCAACGGGTTTGCCGCCAGAACACAGGTGTCGTGAAAAG
AGTTTAGCGGAGTGGAGAAGAGCGGAGCCGAGCCTAGCAGAGACGA
GCCGAGATG

SEQ ID NO: 176—Illustrative nucleotide sequence 5 containing improved spacer, optimal (overlapping) tbs ([KAGNN]₈)-Kozak junction

ATAGCAGAGACGGCTGAGTTTAGCGGAGTGGAGAAGAGCGGAGCCG
AGCCGAGATG

SEQ ID NO: 177—Illustrative nucleotide sequence 6 containing L33 Improved leader tbs ([KAGNN]₁₁)-MCS-Kozak

CTTTTTCGCAACGGGTTTGCCGCCAGAACACAGGAGTTTAGCGGAG
TGGAGAAGAGCGGAGCCGAGCCTAGCAGAGACGAGAAGAGCTCTAG
ACCATG

SEQ ID NO: 178—Illustrative nucleotide sequence 7 containing improved spacer, tbs ([KAGNN]₁₁)-MCS-Kozak

ATAGCAGAGACGGCTGAGTTTAGCGGAGTGGAGAAGAGCGGAGCCG
AGCCTAGCAGAGACGAGAAGAGCTCTAGACCATG

In one embodiment, the nucleotide sequence comprises any one of SEQ ID NO: 172-178.

In one embodiment, the nucleotide sequence comprises:
(a) (i) SEQ ID NO: 101 or 102; and/or

### (ii) any one of SEQ ID NOs: 151-157;

(b) any one of SEQ ID NOs: 127-130, 132-136, 140, 141; and
(c) (i) any one of SEQ ID NOs: 142-149, preferably any one of SEQ ID NOs: 147-149; or
   (ii) any one of SEQ ID NOs: 158-171, preferably any one of SEQ ID NOs: 165-171.

In one embodiment, the nucleotide sequence comprises:
(a) SEQ ID NO: 101 or 102;
(b) any one of SEQ ID NOs: 127-130, 133-136, 140, 141; and
(c) any one of SEQ ID NOs:142-149, preferably any one of SEQ ID NOs: 147-149.

In one embodiment, the nucleotide sequence comprises:
(a) any one of SEQ ID NOs: 151-157;
(b) any one of SEQ ID NOs: 127-130, 133-136, 140, 141; and
(c) any one of SEQ ID NOs: 142-149, preferably any one of SEQ ID NOs: 147-149.

In one embodiment, the nucleotide sequence comprises:
(a) SEQ ID NO: 101 or 102;
(b) any one of SEQ ID NOs: 127-130, 132-136, 140, 141; and
(c) any one of SEQ ID NOs: 157-171, preferably any one of SEQ ID NOs: 165-171.

In one embodiment, the nucleotide sequence comprises:
(a) any one of SEQ ID NOs: 151-157;
(b) any one of SEQ ID NOs: 127-130, 132-136, 140, 141; and
(c) any one of SEQ ID NOs: 157-171, preferably any one of SEQ ID NOs: 165-171.

The practice of the present invention will employ, unless otherwise indicated, conventional techniques of chemistry, molecular biology, microbiology and immunology, which are within the capabilities of a person of ordinary skill in the art. Such techniques are explained in the literature. See, for example, J. Sambrook, E. F. Fritsch, and T. Maniatis (1989) Molecular Cloning: A Laboratory Manual, Second Edition, Books 1-3, Cold Spring Harbor Laboratory Press; Ausubel, F. M. et al. (1995 and periodic supplements) Current Protocols in Molecular Biology, Ch. 9, 13, and 16, John Wiley & Sons, New York, NY; B. Roe, J. Crabtree, and A. Kahn (1996) DNA Isolation and Sequencing: Essential Techniques, John Wiley & Sons; J. M. Polak and James O′D. McGee (1990) In Situ Hybridization: Principles and Practice; Oxford University Press; M. J. Gait (ed.) (1984) Oligonucleotide Synthesis: A Practical Approach, IRL Press; and, D. M. J. Lilley and J. E. Dahlberg (1992) Methods of Enzymology: DNA Structure Part A: Synthesis and Physical Analysis of DNA Methods in Enzymology, Academic Press. Each of these general texts is herein incorporated by reference.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

General Molecular/Cell Biology Techniques and Assays

Modified U1 snRNA Expression Constructs

The DNA-based expression constructs for the modified U1 snRNAs comprise the conserved sequences in the endogenous U1 snRNA gene driving RNA transcription and termination, highlighted below in the non-limiting example of the 256U1 (also referred to as U1_256) snRNA:

(SEQ ID NO: 15)
TAAGGACCAGCTTCTTTGGGAGAGAACAGACGCAGGGGGGGGAGGG
AAAAAGGGAGAGGCAGACGTCACTTCCCCTTGGCGGCTCTGGCAGC
AGATTGGTCGGTTGAGTGGCAGAAAGGCAGACGGGGACTGGGCAAG
GCACTGTCGGTGACATCACGGACAGGGCGACTTCTATGTAGATGAG
GCAGCGCAGAGGCTGCTGCTTCGCCACTTGCTGCTTCACCACGAAG
GAGTTCCCGTGCCCTGGGAGCGGGTTCAGGACCGCTGATCGGAAGT
GAGAATCCCAGCTGTGTGTCAGGGCTGGAAAGGGCTCGGGAGTGCG
CGGGGCAAGTGACCGTGTGTGTAAAGAGTGAGGCGTATGAGGCTGT
GTCGGGGCAGAGGCCCAAGATCTCatttgccgtgcgcgcttGCAGG
GGAGATACCATGATCACGAAGGTGGTTTTCCCAGGGCGAGGCTTAT
CCATTGCACTCCGGATGTGCTGACCCCTGCGATTTCCCCAAATGTG
GGAAACTCGACTGCATAATTTGTGGTAGTGGGGGACTGCGTTCGCG
CTTTCCCCTGGTTTCAAAAGTAGACTGTACGCTAAGGGTCATATCT
TTTTTTGTTTTGGTTTGTGTCTTGGTTGGCGTCTTAAATGTTAA

Key: Upper case only=U1 PolII promoter (nt1-392); lower case=retargeting region (nt393-409); lower case bold=retargeting sequence [in this example targeting nt256-270 of wild type HIV-1 packaging signal] (nt395-409); upper case italics=main U1 snRNA sequence [clover-leaf] (nt410-562); upper case underlined=transcription termination region (nt563-652).

A summary of the initial modified U1 snRNAs and controls used in the study is presented in the table below, indicating the new annealing sequence and the target site sequence (sequences are represented in the 5′ to 3′ direction).

TABLE I
A list of sequences describing the target-annealing sequences
(heterologous sequence that is complementary to the target
sequence) within test modified U1 snRNAs and control U1 snRNAs, and
their target sequences used in the initial study. Nucleotides are
presented as DNA as they would be encoded within their respective
expression cassettes at the ‘retargeting region’. The (AT) motif
was present in all initial constructs, which forms the first two
nucleotides of the U1 snRNA molecule in each case. The target
sequence numbers refer to targets in the NL4-3 (GenBank: M19921.2)
or HXB2 (GenBank: K03455.1) strains of HIV-1 where denoted, since
the lentiviral vector genome in this study contained a hybrid
packaging signal composed of these two highly conserved strains
(packaging sequence used in this study is most similar to the
vector sequence in GenBank: MH782475.1)

| Modified U1 | HIV-1 target | U1 snRNA target- |
|---|---|---|
| snRNA* | sequence [NL4-3]** | annealing sequence |
| U1_16 | 16-GACCAGATCTGAGCC-30 | (AT)GGCTCAGATCTGGTC |
| | (SEQ ID NO: 16) | (SEQ ID NO: 17) |
| U1_31 | 31-TGGGAGCTCTCTGGC-45 | (AT)GCCAGAGAGCTCCCA |
| | (SEQ ID NO: 18) | (SEQ ID NO: 19) |
| U1_76 | 76-TAAAGCTTGCCTTGA-90 | (AT)TCAAGGCAAGCTTTA |
| | (SEQ ID NO: 20) | (SEQ ID NO: 21) |
| U1_136 | 136-TAGAGATCCCTCAGA-150 | (AT)TCTGAGGGATCTCTA |
| | (SEQ ID NO: 22) | (SEQ ID NO: 23) |
| U1_179 | 179-GCAGTGGCG-187 | (AT)CGCCACTGC |
| (9 nt) | (SEQ ID NO: 24) | (SEQ ID NO: 25) |
| U1_181 | 181-AGTGGCGCCCGAACA-195 | (AT)TGTTCGGGCGCCACT |
| | (SEQ ID NO: 26) | (SEQ ID NO: 27) |
| U1_196 | 196-GGGACTTGAAAGCGA-210 | (AT)TCGCTTTCAAGTCCC |
| | (SEQ ID NO: 28) | (SEQ ID NO: 29) |
| U1_211 | 211-AAGggAAaCCAGAGG-225 | (AT)CCTCTGGTTTCCCTT |
| | (SEQ ID NO: 30) | (SEQ ID NO: 31) |
| U1_226 | 226-AGcTCTCTCGACGCA-240 | (AT)TGCGTCGAGAGAGCT |
| U1_241 | 241-GGACTCGGCTTGCTG-255 | (AT)CAGCAAGCCGAGTCC |
| | (SEQ ID NO: 32) | (SEQ ID NO: 33) |
| U1_256 | 256-AAGCGCGCACGGCAA-270 | (AT)TTGCCGTGCGCGCTT |
| | (SEQ ID NO: 34) | (SEQ ID NO: 35) |
| U1_271 | 271-GAGGCGAGGGGCGGC-285 | (AT)GCCGCCCCTCGCCTC |
| | (SEQ ID NO: 36) | (SEQ ID NO: 37) |
| U1_286 | 286-GACTGGTGAGTACGC-300 | (AT)GCGTACTCACCAGTC |
| | (SEQ ID NO: 38) | (SEQ ID NO: 39) |
| U1_305 | 305-AATTTTGAC(TA)-313/5 | (AT)GTCAAAATT |
| (9 nt) | (SEQ ID NO: 40) | (SEQ ID NO: 41) |
| U1_305 | 305-AATTTTGACTAGCGG-319 | (AT)CCGCTAGTCAAAATT |
| | (SEQ ID NO: 42) | (SEQ ID NO: 43) |
| U1_316 | 316-GCGGAGGCTAGAAGG-330 | (AT)CCTTCTAGCCTCCGC |
| | (SEQ ID NO: 44) | (SEQ ID NO: 45) |
| U1_331 | 331-AGAGAGATGGGTGCG-345 | (AT)CGCACCCATCTCTCT |
| | (SEQ ID NO: 46) | (SEQ ID NO: 47) |
| U1_346 | 346-AGAGCGTCgGTATTA-360 | (AT)TAATACTGACGCTCT |
| | (SEQ ID NO: 48) | (SEQ ID NO: 49) |
| U1_361 | 361-AGCGGGGGAGAATTA-375 | (AT)TAATTCTCCCCCGCT |
| | (SEQ ID NO: 50) | (SEQ ID NO: 51 |
| U1_376 | 376-GATCGCGATGGGAAA-390 | (AT)TTTCCCATCGCGATC |
| | (SEQ ID NO: 52) | (SEQ ID NO: 53) |
| U1_391 | 389-AAATTCGGTTAAGGC-403 | (AT)GCCTTAACCGAATTT |
| | (SEQ ID NO: 54) | (SEQ ID NO: 55) |
| U1_690 | 7159-GATCTTCAGACCTGG-7173 | (AT)CCAGGTCTGAAGATC |
| | (SEQ ID NO: 56) | (SEQ ID NO: 57) |
| U1_1203 | 7672-TTACACAAGCTTAAT-7686 | (AT)ATTAAGCTTGTGTAA |
| | (SEQ ID NO: 58) | (SEQ ID NO: 59) |
| U1_1546 | 4375-TAGTAGACATAATAG-4389 | (AT)CTATTATGTCTACTA |
| | (SEQ ID NO: 60) | (SEQ ID NO: 61) |
| Control U1 | | U1 snRNA target- |
| snRNA | Target sequence | annealing sequence |
| U1_LacZ1 | 388-CTACAGGAA-396 | (AT)TTCCTGTAG |
| | (SEQ ID NO: 62) | (SEQ ID NO: 63) |
| U1_LacZ2 | 438-TCATCTGTG-446 | (AT)CACAGATGA |
| | (SEQ ID NO: 64) | (SEQ ID NO: 65) |

*numbering relative to vector genome RNA sequence
**lower case target sequence is for (HXB2), underlined target sequence is an AA > CGCG frameshift in the gag ORF (U1 376)

Adherent Cell Culture, Transfection and Lentiviral Vector Production

HEK293T cells were maintained in complete media (Dulbecco's Modified Eagle Medium (DMEM) (Sigma) supplemented with 10% heat-inactivated (FBS)(Gibco), 2 mM L-glutamine (Sigma) and 1% non-essential amino acids (NEAA) (Sigma)), at 37° C. in 5% CO₂.

The standard scale production of HIV-1 vectors in adherent mode was in 10 cm dishes under the following conditions (all conditions were scaled by area when performed in other formats): HEK293T cells were seeded at 3.5×10 5 cell per ml in 10 mL complete media and approximately 24 hours later the cells were transfected using the following mass ratios of plasmids per 10 cm plate: 4.5 μg Genome, 1.4 μg Gag-Pol, 1.1 μg Rev, 0.7 μg VSV-G and between 0.01 and 2 μg of modified U1 snRNA plasmid.

Transfection was mediated by mixing DNA with Lipofectamine 2000CD in Opti-MEM as per manufacturer's protocol (Life Technologies). Sodium butyrate (Sigma) was added ˜18 hours later to 10 mM final concentration for 5-6 h, before 10 ml fresh serum-free media replaced the transfection media. Typically, vector supernatant was harvested 20-24 hours later, and then filtered (0.22 μm) and frozen at −20/−80° C. As a positive control for nuclease treatment, typically Benzonase® was added to the harvests at 5U/mL for 1 hour prior to filtration.

Suspension Cell Culture, Transfection and Lentiviral Vector Production

HEK293T.1-65s suspension cells were grown in Freestyle +0.1% CLC (Gibco) at 37° C. in 5% CO₂, in a shaking incubator (25 mm orbit set at 190 RPM). All vector production using suspension was carried out in 24-well plates (1 mL volumes, on a shaking platform), 25 mL shake flasks or in bioreactors (≤5 L). HEK293 Ts cells were seeded at 8×10⁵ cells per ml in serum-free media and were incubated at 37° C. in 5% CO₂, shaking, throughout vector production. Approximately 24 hours after seeding the cells were transfected using the following mass ratios of plasmids per effective final volume of culture at transfection: 0.95 μg/mL Genome, 0.1 μg/mL Gag-Pol, 0.6 μg/mL Rev, 0.7 μg/mL VSV-G, and between 0.01 to 0.2 μg/mL modified U1 snRNA plasmid.

Transfection was mediated by mixing DNA with Lipofectamine 2000CD in Opti-MEM as per manufacturer's protocol (Life Technologies). Sodium butyrate (Sigma) was added ˜18 hrs later to 10 mM final concentration. Typically, vector supernatant was harvested 20-24 hours later, and then filtered (0.22 μm) and frozen at −20/−80° C. As a positive control for nuclease treatment, typically Benzonase® was added to the harvests at 5U/mL for 1 hour prior to filtration.

Lentiviral Vector Titration Assays

For lentiviral vector titration by GFP marker-containing cassette, HEK293T cells were seeded at 1.2×10⁴ cells/well in 96-well plates. GFP-encoding viral vectors were used to transduce the cells in complete media containing 8 mg/ml polybrene and 1× Penicillin Streptomycin for approximately 5-6 hours after which fresh media was added. The transduced cells were incubated for 2 days at 37° C. in 5% CO₂. Cultures were then prepared for flow cytometry using an Attune-NxT (Thermofisher). Percent GFP expression was measured and vector titres were calculated using a predicted cell count of 2×10⁴ cells at the time of transduction (base on typical growth rate), the dilution factor of the vector sample, the percentage positive GFP population and total volume at transduction.

For lentiviral vector titration by integration titre, 0.5 mL volumes of neat to 1:5 diluted vector supernatants were used to transduce 1×10⁵ HEK293T cells at 12-well scale in the presence of 8 μg/mL polybrene. Cultures were passaged for 10 days (1:5 splits every 2-3 days) before host DNA was extracted from 1×10⁶ cell pellets. Duplex quantitative PCR was carried out using a FAM primer/probe set to the HIV packaging signal (ip) and to RRP1, and vector titres (TU/mL) calculated using the following factors: transduction volume, vector dilution, RRP1-normallised HIV-1 ψ copies detected per reaction.

Transcriptional Read-Through (‘Read-In’) Analysis of LV-Transduced Cells

The HIV vectors indicated were produced by transient transfection of suspension-mode 1.65s cells in the presence or absence of 256_U1 snRNA at 24 well plate scale. Supernatants were harvested after two days before being titrated by GFP expression using flow cytometry on HEK293T cells. GFP titres were then used accordingly to transduce 4.5×10⁴ HEK293T or 92BR cells (Donkey primary fibroblasts) at a multiplicity of infection (MOI) of 1. Transduced cells were passaged three times over ten days prior to being harvested and split into 2 aliquots; one was processed for total RNA extraction and one processed for genomic DNA extraction. 200 ng (293T) or 80 ng (92BR) of total RNA was DNAse I-treated and subjected to RT-PCR using the SSIV VILO RT system (Life Technologies). cDNA was diluted to 1 ng/ul, and 5 ul was subjected to SYBR qPCR using primer sets directed to the HIV Psi and cellular GAPDH. The Delta Ct method was used to normalise HIV Psi copies to GAPDH copies in order to generate expression score between samples. Genomic DNA extracts were prepared using the Qiacube extraction system (Qiagen) and 5 ul of eluted DNA was subjected to HIV vector integration assay using qPCR. HIV Psi copies were normalised to cellular target RPPH1 in order to calculate the average of integrated vector genomes per cell. The HIV RNA expression score of a sample was then normalised to the number of integrated vector copies per cell in order to account for transduction efficiency. This final value is the relative HIV Psi RNA expression score, and for each cell line tested all values were normalised to the standard vector (intact MSD and crSDs) made in the absence of 256_U1 snRNA.

### Example 1—Promiscuous Splicing from the MSD, Reduced Titres of MSD-2KO Lentiviral Vectors and Titre Recovery/Boost by Re-Directed U1 snRNAs

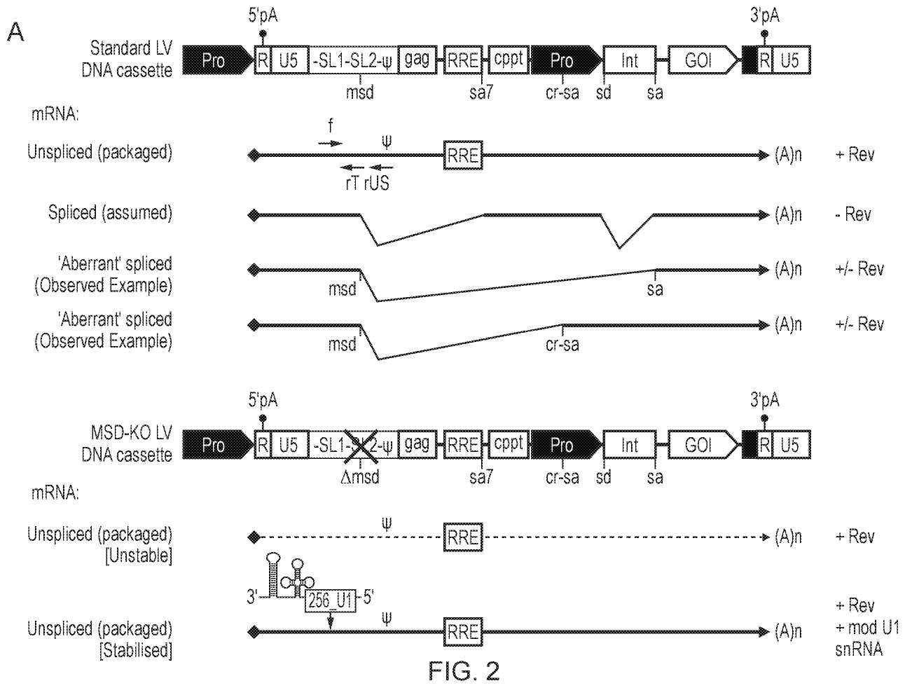

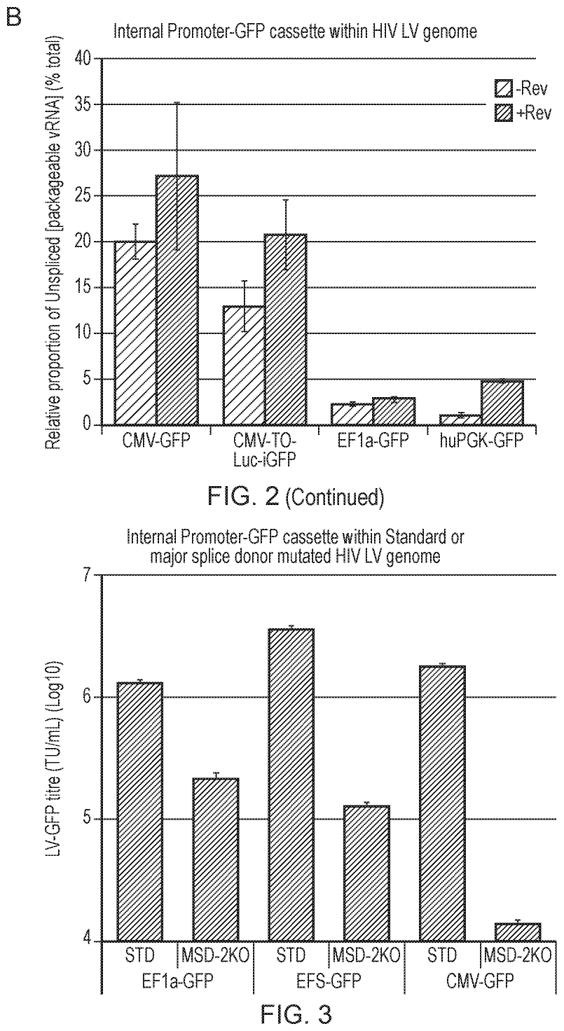

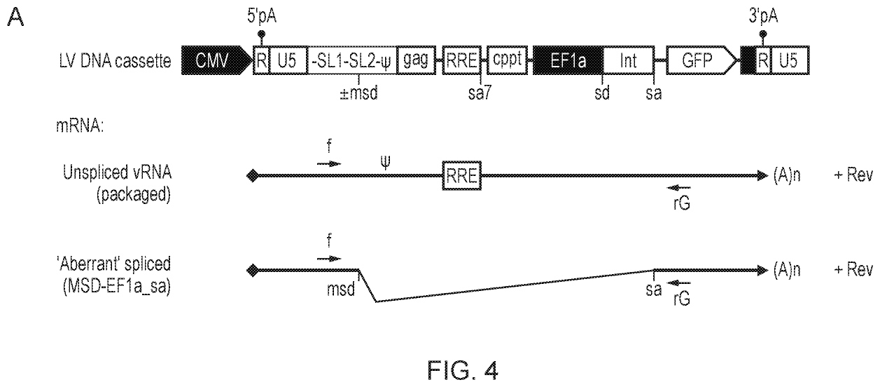

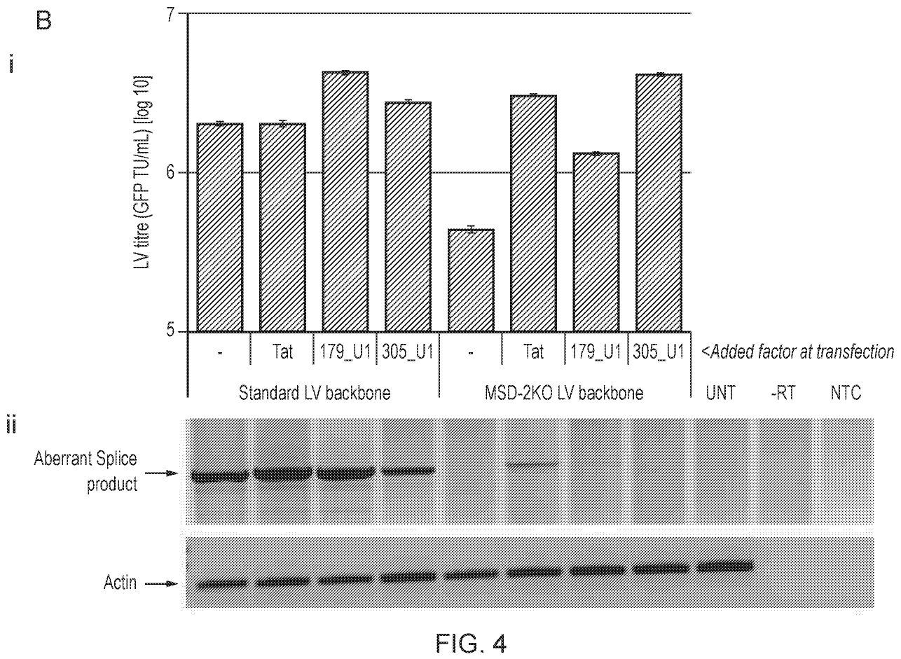

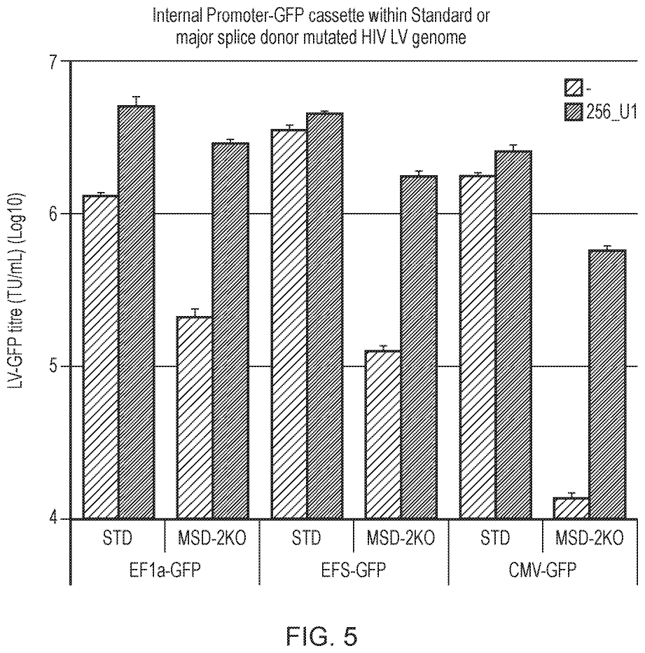

The general architecture of lentiviral vector genomes has remained consistent through all three generations of vector systems (Lentiviral vectors: basic to translational. Toshie SAKUMA, Michael A. BARRY and Yasuhiro IKEDA. Biochem. J. (2012) 443, 603-618), with maintenance of the 5′region of the HIV-1 provirus containing the packaging sequence and the position of the RRE upstream of the transgene cassette, but differed in other aspects, with later generations becoming tat-independent, self-inactivating in the 3′LTR, and incorporating the use of the cppt and wPRE. The apparent lack of examples in engineering of the 5′packaging sequence is likely due to its complex structure and the condensed information encoded within that is necessary for many aspects of HIV-1 replication: transcription, balance in splicing, translation of GagPol, genome dimerization, assembly, reverse transcription, and integration. Within this complex region, the major splice donor (MSD) is embedded within the stem-loop 2 (SL2) region, between SL1 (dimerization loop) and SL3 (binding to Gag). The re-positioning of the RRE sequence (and associated splice acceptor 7 (sa7) within the envelope region) to immediately downstream of the packaging region in lentiviral vector genomes was thought to ‘offer’ the MSD a splice acceptor (sa7) in the absence of rev. It was assumed that during lentiviral vector production the supply of rev results in rev binding to the RRE and suppression of MSD splicing to sa7, and consequently production of unspliced, full length lentiviral vector genomic vRNA (FIG. 2A). However, the present inventors (FIG. 4Bii) and others (e.g. Cui et al. (1999), J. Virol., 73: 6171-6176) show that aberrant splicing from the MSD to splice acceptor sites within the transgenic sequences can be substantial, leading to relatively modest levels of unspliced vRNA (relative to total—see FIG. 2B) available for packaging into vector virions—in some cases less than 5%.

The inefficiencies in generating vRNA for packaging may not always be directly observed in lentiviral vector titres produced in transient transfection methods because of the delivery of the high numbers of vector genome plasmids to cells; titres of standard 3^{rd} generation vectors above 1×10⁷ TU/mL are routinely possible, even with this type of aberrant splicing occurring. However, the present inventors anticipate that for development of stable producer cell lines, where much lower numbers of integrated vector genome cassettes may be present, the issue of aberrant splicing will likely be more substantial in effect. Indeed, the present inventors typically find that the genome component is limiting in stable producer clones, and hypothesise that MSD activity may substantially contribute to this limitation.

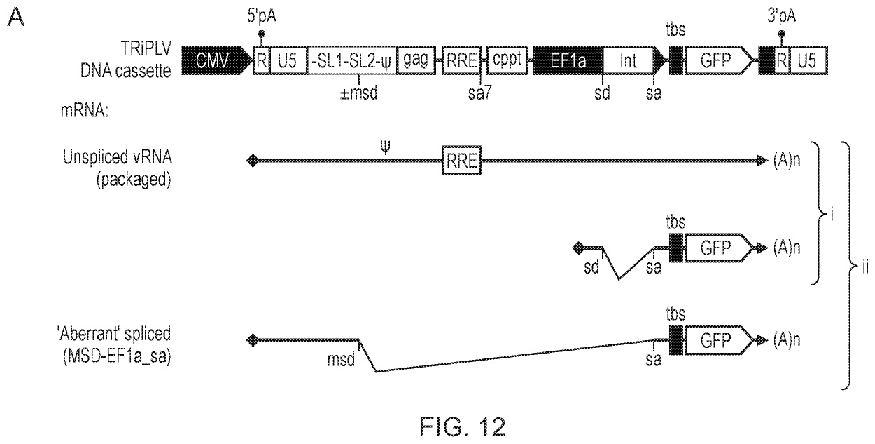

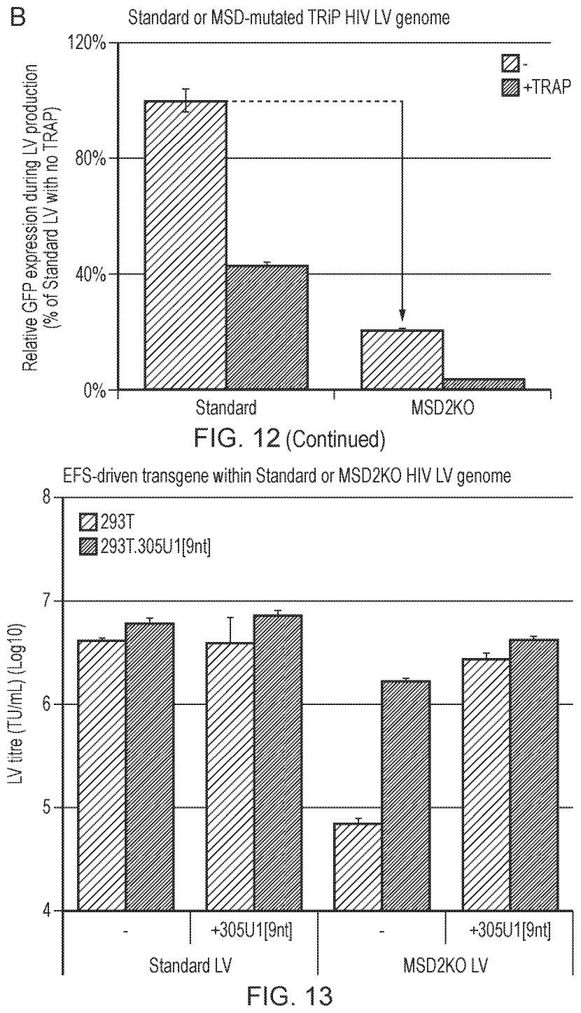

There is a further perhaps less obvious consequence in generating aberrantly spliced mRNA resulting from the MSD into transgene cassettes: (increased) expression of the transgene cassette during production. Previously, the TRiP system was developed to repress transgene expression during lentiviral vector production (described in WO 2015/092440), which enables recoveries in vector titres that are linked in proportion to a negative effect of the specific transgene protein on vector production. The present inventors have found that efficient aberrant splicing (for example in standard lentiviral vectors containing EF1a driven cassettes—see FIGS. 4 and 12) produces mRNAs that typically encode for the transgene. Without wishing to be bound by theory, for EF1a driven cassettes the MSD splices to the strong EF1a splice acceptor but for other promoter-UTR sequences the MSD ‘selects’ weaker cryptic splice acceptors, even in the presence of rev. The MSD appears to ‘look past’ the RRE-sa7 sequence in favour of other sites more central to the vector genome i.e. in the transgene promoter region. This may be a ‘residual’ property of the HIV-1 5′packaging region since in wild type HIV-1 the MSD typically splices to splice acceptors placed centrally and to the 3′end of the genome. It is possible that the MSD aberrantly splices at many places within the vector sequences downstream but that only mRNAs that pass nonsense-mediated decay rules (i.e. they appear to be legitimate mRNAs because they encode a protein [the transgene protein]) are transported to the cytoplasm (and/or are stable in the cytoplasm) where they are then translated. This creates additional burden on the TRiP system to maintain repression of transgene encoding mRNA, leading to less repressive control over a larger pool of mRNA (see FIG. 12). Moreover, it is likely that the use of tissue specific promoters (partly to avoid transgene expression during lentiviral vector production) will be ‘undone’ by the cytoplasmic appearance of translatable mRNA encoding the transgene by this mechanism of aberrant splicing. In essence, the transgene will be expressed by the (typically powerful) constitutive promoter that is driving the expression of the vector genome vRNA.

Therefore, there are many reasons to generate MSD-mutated lentiviral vectors, and indeed others have attempted to do so in tat-independent lentiviral vectors without success. The present inventors have found that mutation of the MSD in HIV-1 tat-independent 3^{rd} generation vectors activates an adjacent cryptic splice donor site within SL2, resulting in substantial levels of splicing. For this reason the present inventors have employed mutations in both the MSD and the nearby cryptic spice donor (crSD) (see FIG. 10), and refer to this modification as ‘MSD-2KO’ or ‘MSD2KO’ or ‘functional modification of the MSD’. It is shown in FIG. 4 that this double mutation is extremely effective in ablating aberrant splicing from the splicing region of SL2 (including both MSD and crSD) to the strong EF1a splice acceptor during lentiviral vector production. It is also shown that the MSD-2KO lentiviral vector genome containing three different promoter-GFP transgene cassettes leads to reduced vector titres (FIG. 3), as similarly reported by others. In FIG. 4Bi, it is also demonstrated that provision of HIV-1 tat in trans is able to rescue the observed reduction in titre of the MSD-2KO lentiviral vector genome, although the amount of ‘aberrant’ splice product (from a minor cryptic splice donor in SL4) is increased (FIG. 4Bii). Importantly, it is shown that modified U1 snRNAs re-directed to different regions of the vector packaging signal increase MSD-2KO lentiviral vector genome titres, and without increasing the presence of the minor splice product (FIG. 4).

### Example 2—Enhancement of MSD-Mutated Lentiviral Vector Titres is not Due to Suppression of the 5′polyA Site within Vector Genome Cassettes

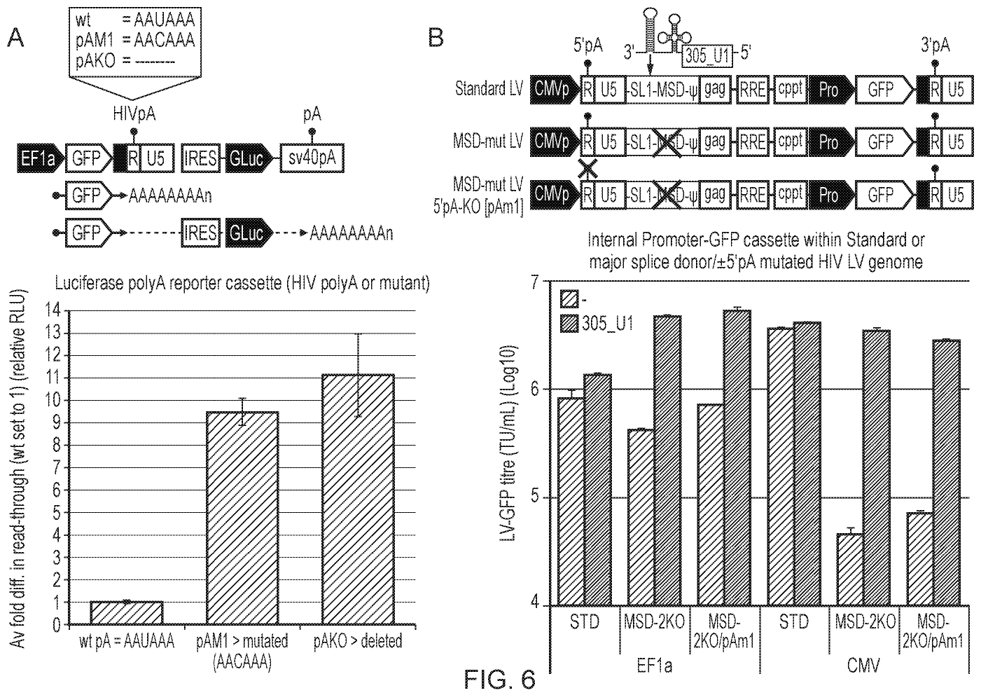

To assess if the present invention is acting to suppress the 5′polyA site, a functional mutation to the 5′polyA site was introduced within MSD-2KO lentiviral vector genomes, containing either EF1a or CMV driven GFP transgene cassettes (FIG. 6); the description of the ‘pAm1’ polyA mutation is in FIG. 6A, which demonstrates complete abolition polyadenylation activity. Surprisingly, the present inventors found that mutation of the 5′polyA signal only partially increased titres in the EF1a-GFP containing MSD-2KO lentiviral vector genome, and had virtually no effect in the CMV-GFP MSD2KO lentiviral vector genome, which seemed to be commensurate with the degree of attenuating effect of the MSD-2KO mutation (for EF1a-containing genomes, the MSD2KO mutation is less pronounced). Importantly, the supply of the 305U1 molecule in this experiment increased titres of both the standard lentiviral vector genomes (in which endogenous U1 snRNA is presumably capable of fully suppressing any potential residual 5′polyA activity) and the MSD2KO/pAm1 lentiviral vector genomes, which had no possible 5′polyA activity. This provides compelling evidence that the modified U1 snRNA, supplied to recover titres of MSD-mutated lentiviral vectors, is acting at a post-transcriptional step not previously described.

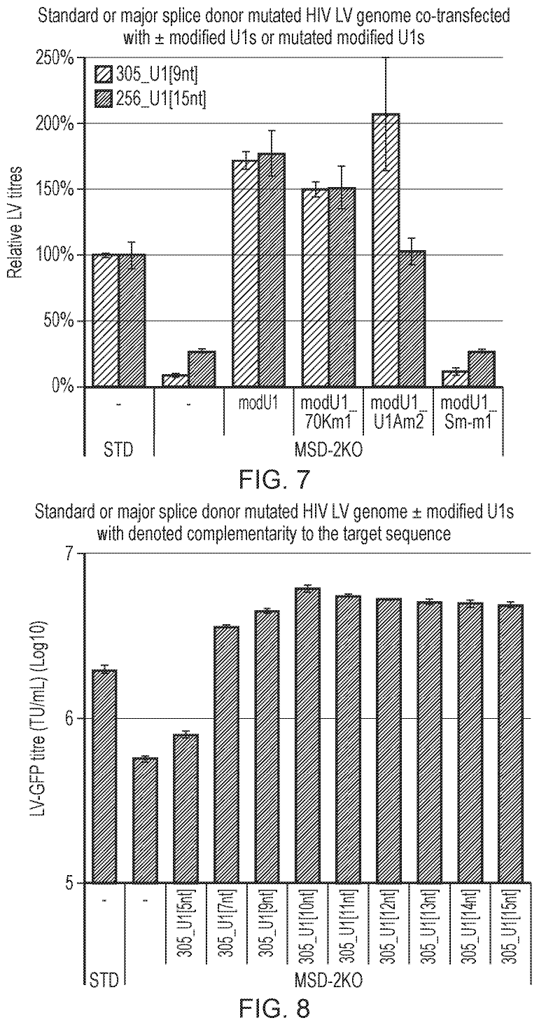

The present inventors then sought to mutate the 70K and U1A binding loops of the 305U1 and 256U1 in order to evaluate if this impacted the observed increase in titres of MSD2KO lentiviral vector genomes. FIG. 7 demonstrates that functional mutation of SL1 or SL2 within modified U1 snRNA has no effect on the ability of these molecules to augment MSD-2KO lentiviral vector titres when co-expressed during production; only the Sm protein binding mutation blocked this activity. This shows that the previously imperative 70K-binding properties of re-directed U1 snRNAs in suppressing polyA activity is not important in the invention, and provides further evidence that modified U1 snRNAs used to increase MSD-mutated lentiviral vector titres are functioning by a novel mechanism.

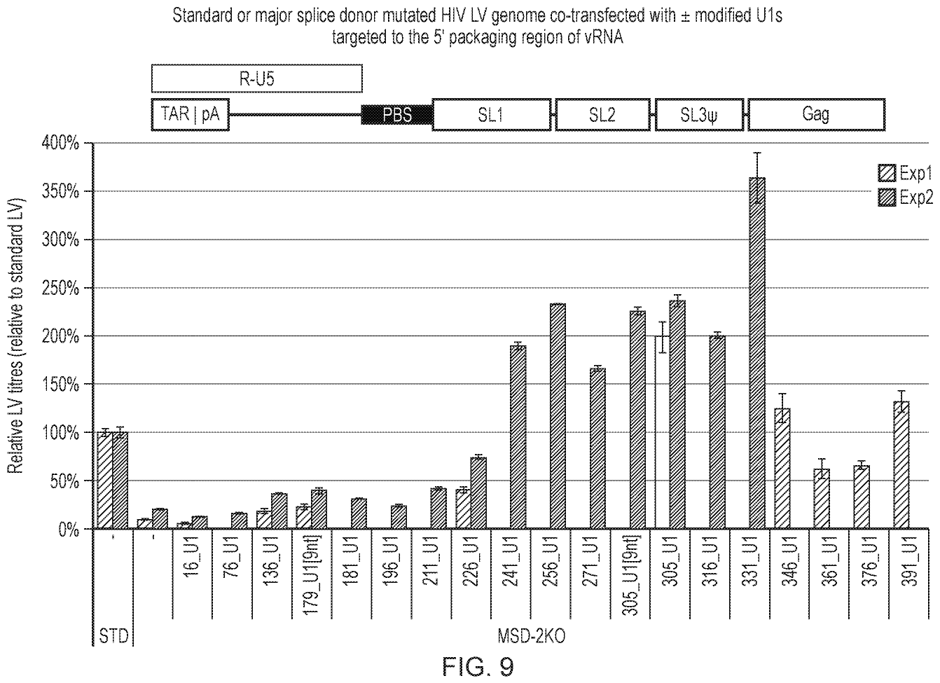

To assess if the titre increase mediated by the modified U1 snRNAs when applied to MSD-2KO lentiviral vectors differed in their ‘preferred’ target site compared to standard lentiviral vectors a panel of modified U1 snRNAs that targeted different sites (see Table I) along the 5′ region of the MSD-2KO lentiviral vector genome were screened (FIG. 9). This screen indicates that targeting to the packaging region is preferred (SL1-3), with perhaps a ‘hotspot’ within SL3. The screen was performed with modified U1 snRNA that had 15 nucleotides of complementarity to the target site (or 9 nucleotides were stated), as we had previously demonstrated for standard lentiviral vectors, the increase in titre may be more robust when using complementarity lengths of greater than 9 nucleotides. Indeed, we show that for MSD-2KO lentiviral vectors, the titre boost observed with modified U1 snRNAs (targeting the ‘305’ sequence) could be observed with only 7 nucleotides of complementarity but that in preferred use it would be better to use 10-to-15 nucleotides of complementarity due to an increase in titre boost (FIG. 8), and also because this would minimize any possible ‘off-target’ effects by the modified U1 snRNAs.

### Example 3—Enhancement of MSD-Mutated Lentiviral Vector Titres by Modified U1 snRNAs is not Dependent on the Type of Splice Donor Mutation

FIG. 10A displays the genetic modification to the SL2 loop of the ‘MSD-2KO’ variant of MSD-mutated lentiviral vector genome packaging region, which mutates both the MSD and the cryptic splice donor positioned downstream (the MSD-2KO variant has been utilized in many of the non-limiting examples herein). To assess if the effect in boosting titres by use of the modified U1 snRNAs was in anyway dependent on the specific changes made to the MSD-2KO variant, we made three other splice donor region mutants: [1] ‘MSD2KOv2’, which also introduced two specific changes within the MSD and cryptic donor sequences, [3] ‘MSD-2KOm5’, which replaces the entire SL2 loop with an artificial stem loop, and [3] a complete SL2 deletion, thus removing the entire splice donor region (also termed the splicing region). We then produced standard or MSD-mutated lentiviral vector variants (containing an EFS-GFP expression cassette) in HEK293T cells +/−modified U1 snRNA (256U1), and titrated vector supernatants (FIG. 10B). The results show that all four MSD-mutated lentiviral vector variants were attenuated compared to the standard vector but that all four could be enhanced by use of modified U1 snRNA supplied during lentiviral vector production, indicating no specific sequence dependency of splice donor region mutation by the modified U1 snRNA. Interestingly, the MSD-2KOm5 variant was the least attenuated, and when produced in the presence of the 256U1 molecule gave the greatest increase in output titres, irrespective of the identity of the internal promoter employed (comparing EFS, EF1a, CMV and human PGK promoters).

### Example 4—the Use of a Modified U1 snRNA Cassette Encoded within a Lentiviral Vector Genome Plasmid DNA Backbone in Cis

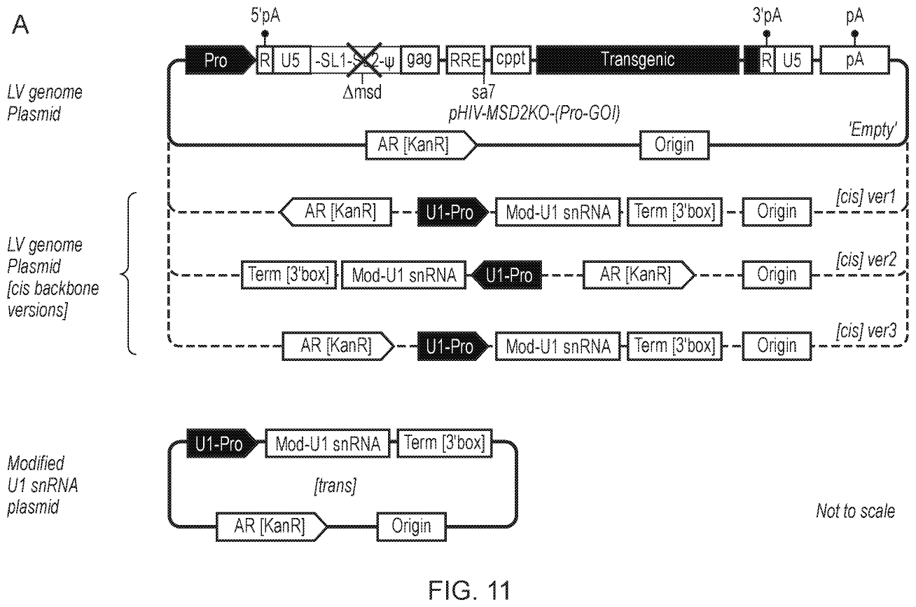

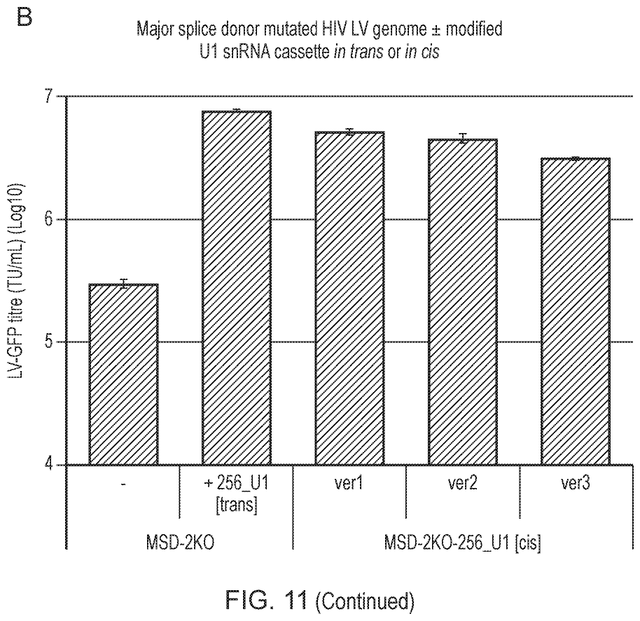

Previous examples herein have disclosed the use of modified U1 snRNA molecules during lentiviral vector production in trans by transient co-transfection of HEK293T cells with lentiviral vector component plasmids and modified U1 snRNA encoding plasmids. To evaluate if the MSD-mutated lentiviral vector genome cassette and modified U1 snRNA cassettes could be suitably encoded within the same plasmid DNA molecule, three variant constructs were cloned (FIG. 11A). An MSD-mutated lentiviral vector genome cassette (the MSD-2KO variant) was modified such that a 256U1 expression cassette was inserted in three different configurations relative to the lentiviral vector genome cassette and/or functional plasmid backbone sequences. These ‘cis’ version plasmids were used to make MSD-mutated lentiviral vectors in HEK293T cells and compared to the ‘trans’ mode, where the modified U1 snRNA plasmid was co-transfected with the unmodified MSD-mutated lentiviral vector genome (FIG. 11B). The results show that the titres of these ‘cis’ version plasmids was similar to the unmodified MSD-2KO lentiviral vector genome +256U1 supplied in co-transfection.

### Example 5—the Use of a Cell Line Stably Expressing a Modified U1 snRNA to Enhance Production of Both Standard or MSD-Mutated Lentiviral Vectors

The 305U1 expression cassette was stably integrated into HEK293T cells, and standard or MSD-2KO lentiviral vectors produced by transient transfection +/−additional 305U1 plasmid DNA. The successful generation of stable cells reveal for the first time that modified U1 snRNA can be expressed endogenously within cells without cyctotoxic effect, indicating that modified U1 snRNAs do not titrate-out cellular factors involved in either U1 snRNA synthesis or the spliceosome, and either no off-targeting occurs or that off-targeting effects do not impact upon normal cell viability. The output titre of lentiviral vectors demonstrate that the titre increase mediated by modified U1 snRNAs on both standard and MSD-2KO lentiviral vectors is possible in stable provision of modified U1 snRNAs (FIG. 13). This will enable modified U1 snRNAs to be easily incorporated into lentiviral vector packaging and producer cell lines.

### Example 6—MSD-Mutated Lentiviral Vectors Produce Less Transgene Protein During Production

A further advantage of ablating aberrant splicing during lentiviral vector production is to reduce the amount of transgene-encoding mRNA that leads to transgene protein production. Transgene expression can impact substantially on lentiviral vector production, which has led us to previously develop the TRiP system to suppress transgene translation during viral vector production (described in WO 2015/092440). In brief, the bacterial protein ‘TRAP’ is co-expressed during vector production and binds to its ‘TRAP binding sequence’ (tbs) inserted upstream of the transgene ORF within the 5′UTR—thus blocking the scanning ribosome.

During the course of this work, we unexpectedly found that transgene-encoding mRNAs were effectively produced from the ‘external’ (CMV) promoter driving the vector genome cassette due to splicing-out from the major donor splice region of the SL2 to internal splice acceptor sites. The degree to which this occurs depends on the internal sequences between the cppt and the transgene ORF (i.e. the promoter-5′UTR sequence). The use of the EF1a promoter (containing a very strong splice acceptor) in the transgene cassette, results in aberrant splicing from the MSD in over 95% of total transcripts originating from the external promoter (see FIG. 2). By comparing total GFP expression in standard or MSD-2KO lentiviral vector production cultures (FIG. 12B), we show that up to 80% of the transgene protein expressed during production originates from the aberrant splice product. We found that combining the MSD-2KO genotype with the TRiP system augmented the reduction in transgene protein produced.

### Example 7: Optimisation of tbs-Kozak Junction

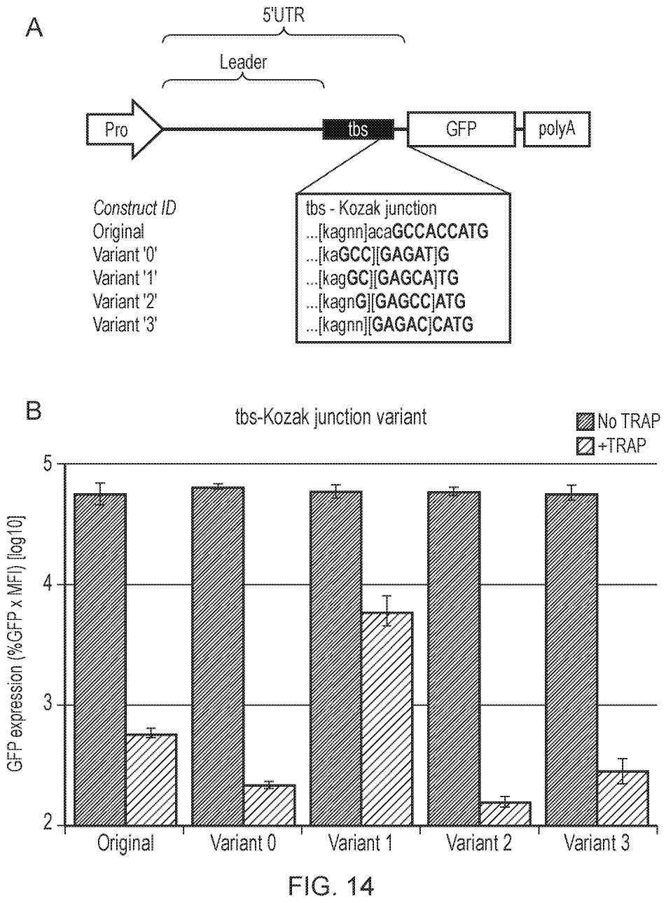

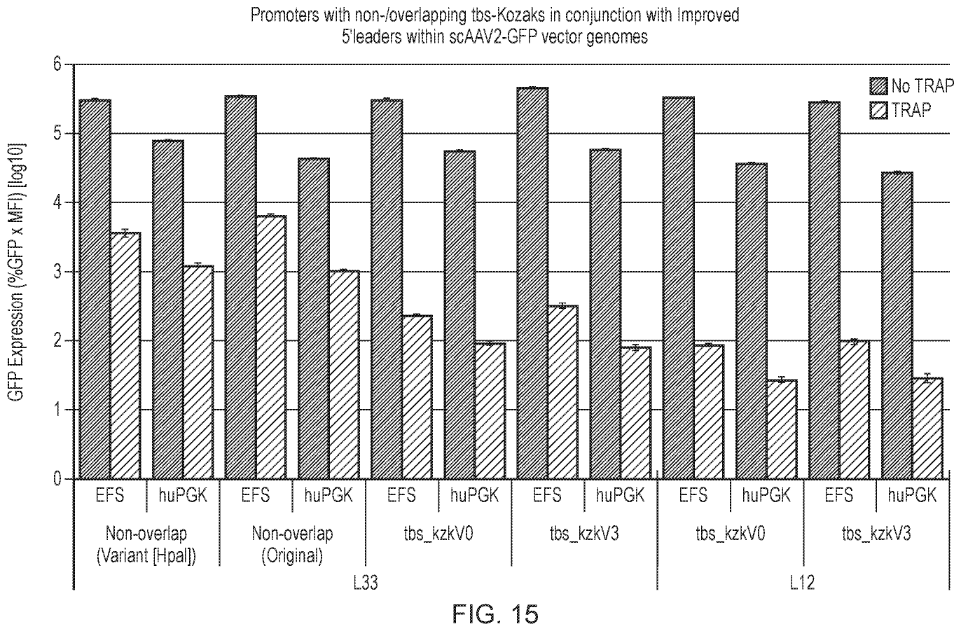

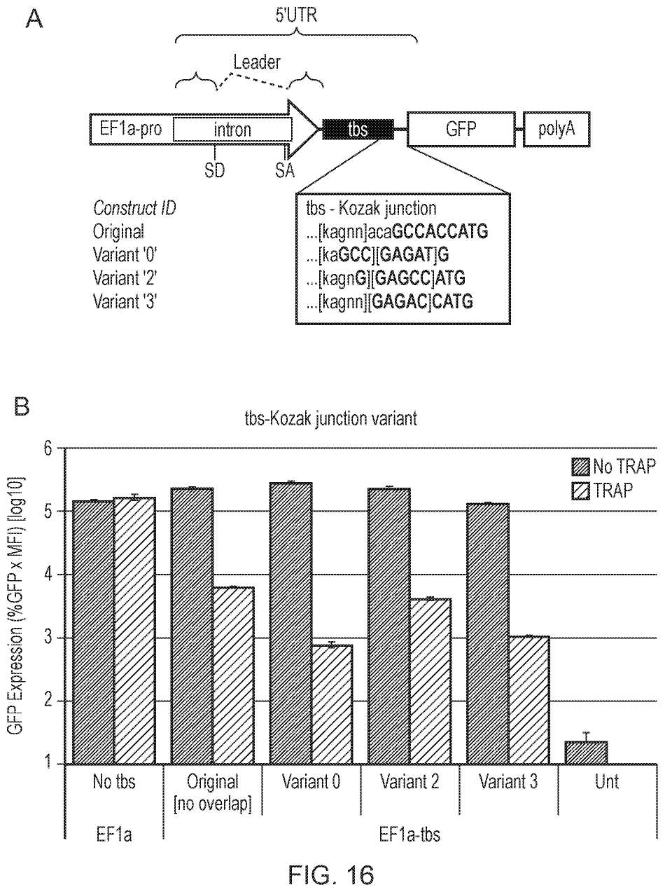

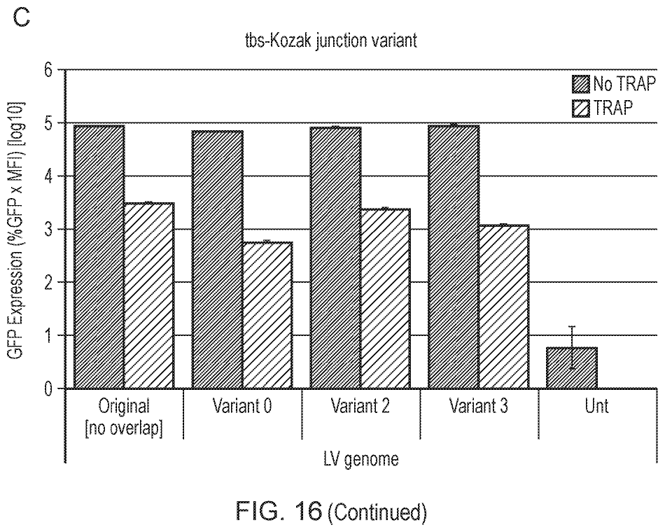

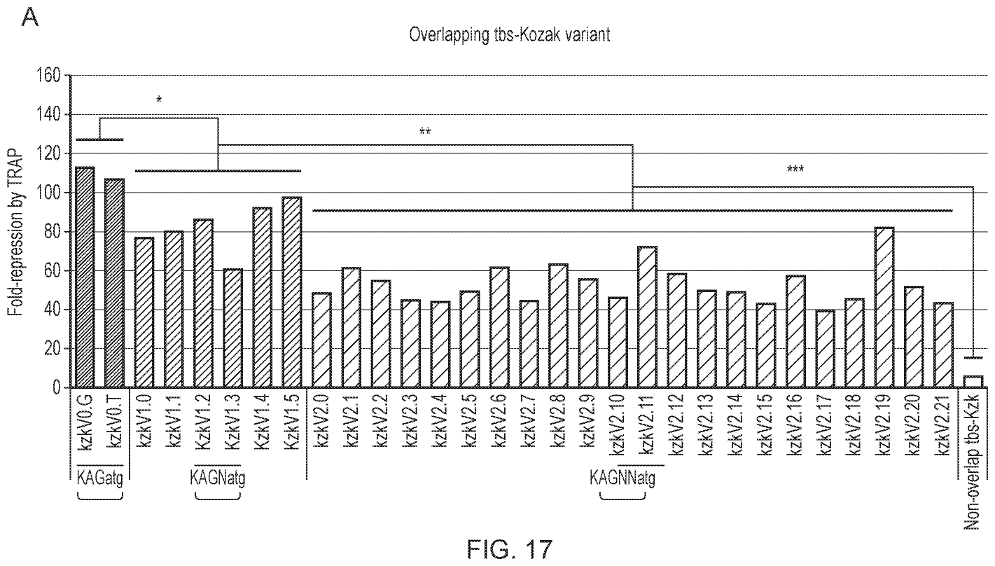

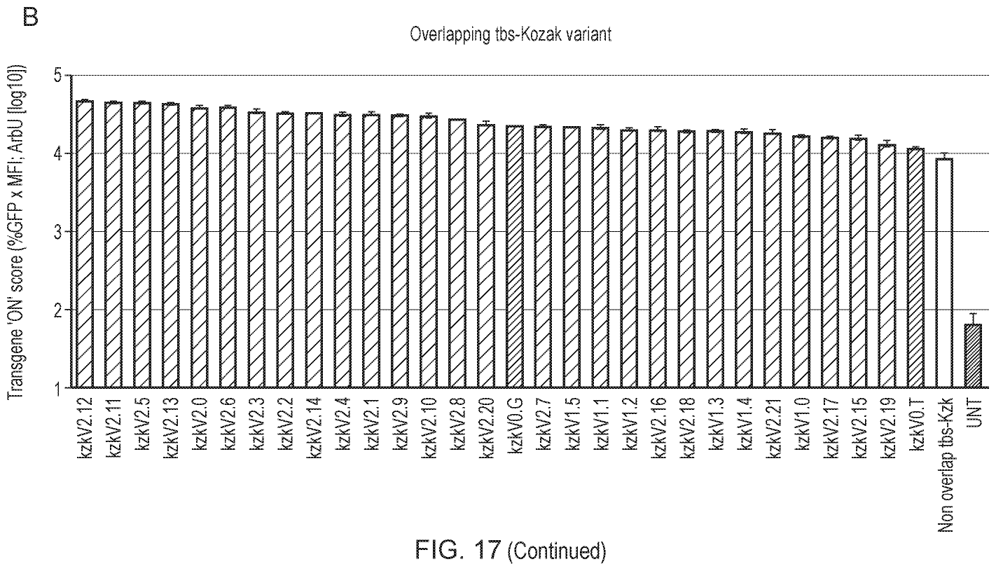

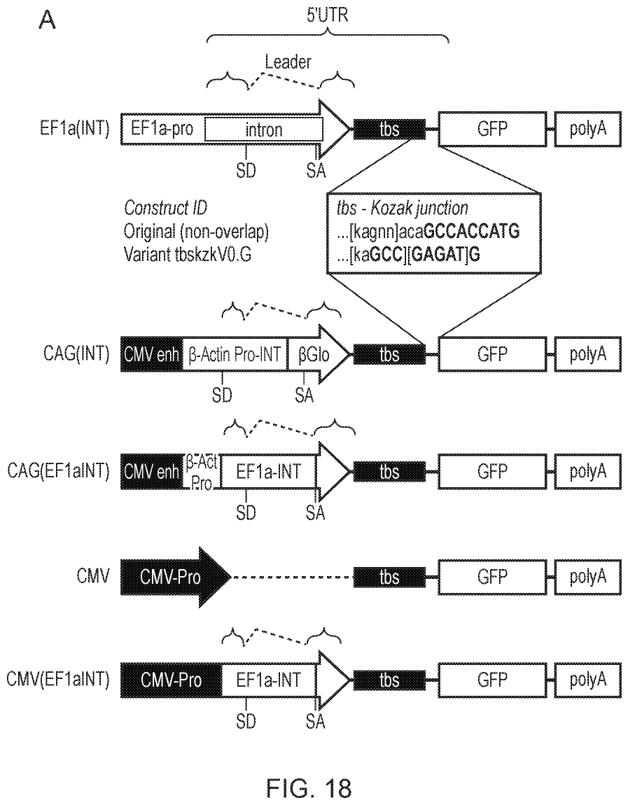

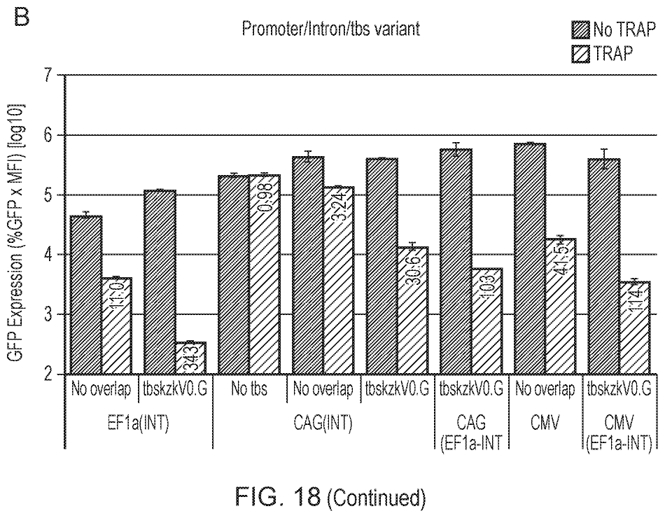

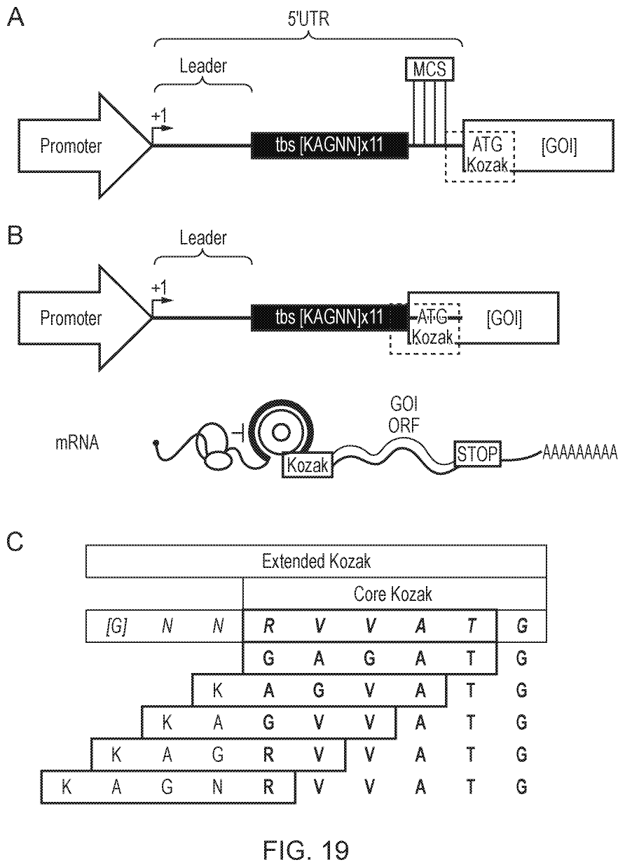

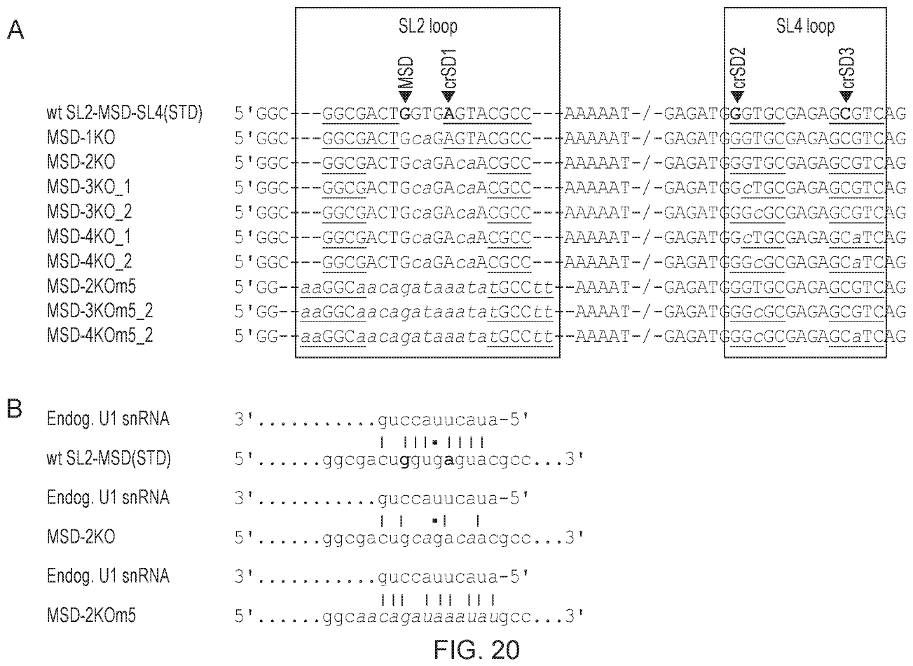

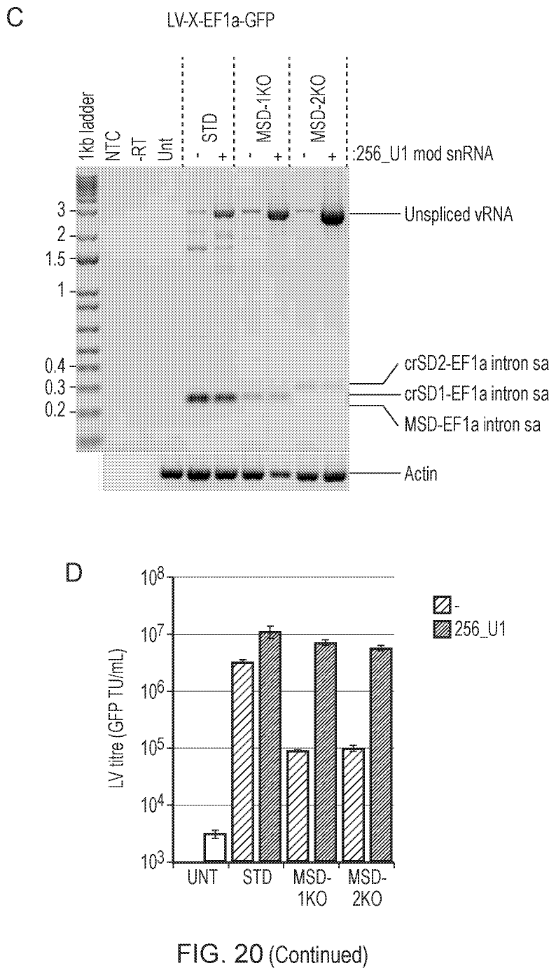

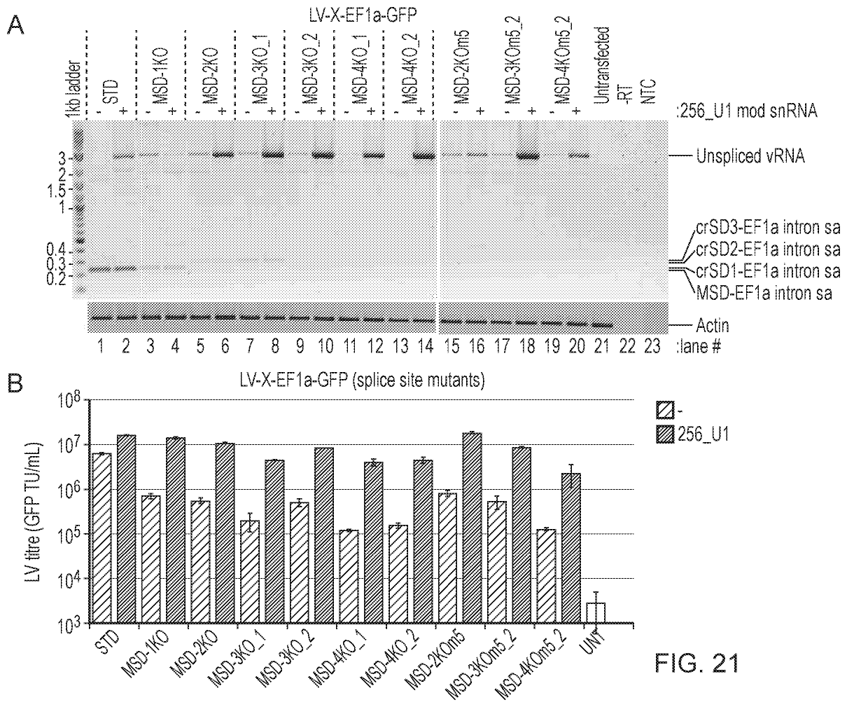

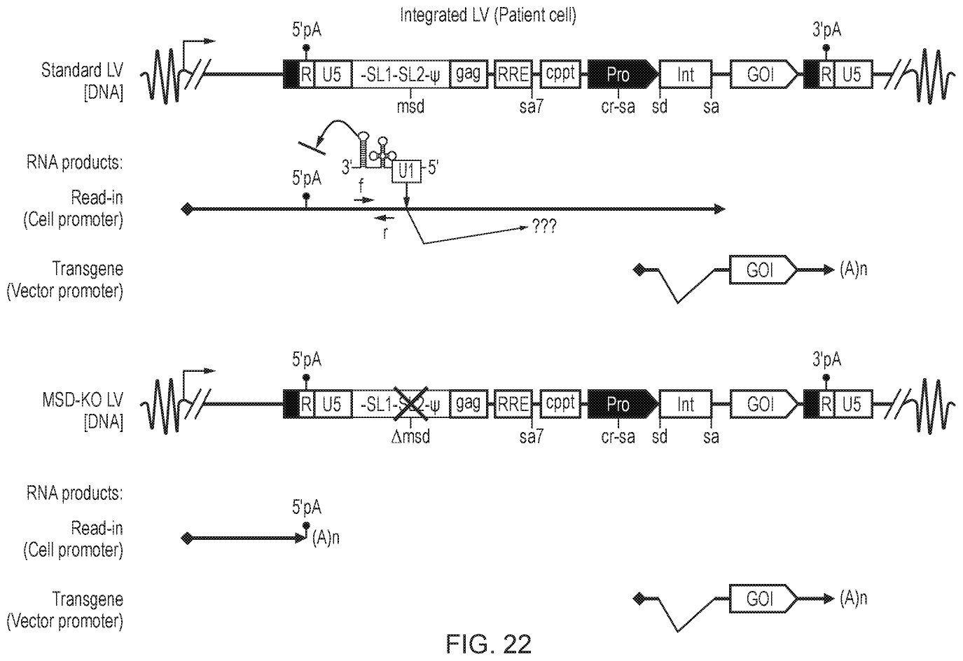

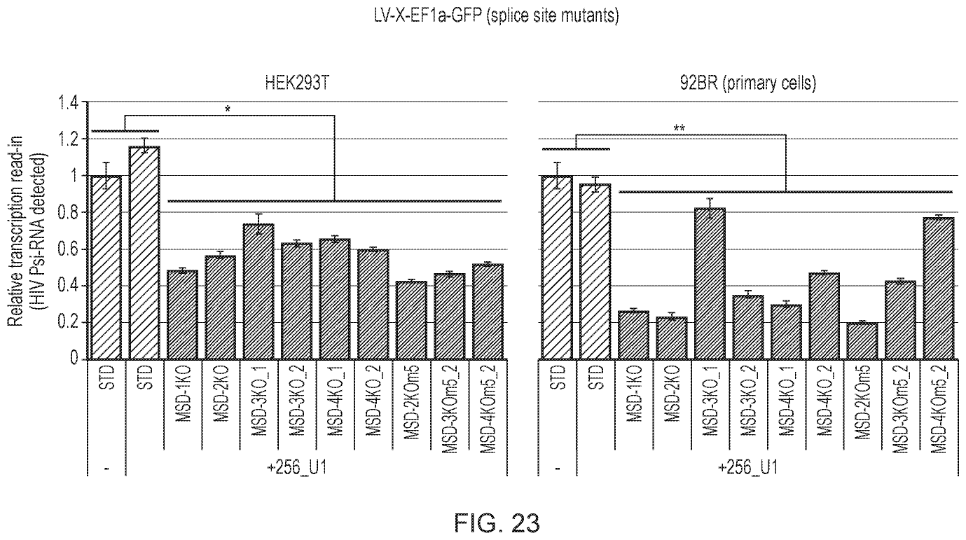

Four tbs-Kozak variants were designed (see Table II-Panel I) and cloned into a GFP reporter construct as indicated in FIG. 14A. Variants 0, 1 and 2 were designed such that the extended Kozak sequence (conforming to consensus GNNRVVATG) overlapped with the final two tbs repeat sequences, whereas Variant 3 overlapped just the final tbs repeat sequence. These variants, along with the original reporter in which the Kozak sequence is 3 nucleotides downstream of the final tbs repeat sequence (i.e. no overlap), were individually co-transfected into HEK293T cells +/−a TRAP-expression plasmid, and GFP expression measured two days post-transfection by flow cytometry. A GFP Expression Score (% GFP positive cells×MFI) was generated from flow cytometry data and plotted (FIG. 14B).

Two of these tbs-Kozak variants were tested in conjunction with two different promoters (EFS and huPGK) within scAAV2 vector genome expression cassettes, and compared to tbs-containing cassettes that did not have overlapping tbs/Kozak sequences. These GFP reporter vector genome plasmids were individually co-transfected into HEK293T cells +/−a TRAP-expression plasmid, and GFP expression measured two days post-transfection by flow cytometry. A GFP Expression Score (% GFP positive cells×MFI) was generated from flow cytometry data and plotted (FIG. 15). The non-overlapping tbs/Kozak variants (Original and a second variant containing an HpaI site between the tbs and the Kozak) were capable of 50-to-100 fold repression, whereas the new tbs-Kozak variants (tbs_V0 and tbs_V3) were repressed by at least 10-times this (500-to-3500 fold). These tbs-Kozak variants also performed similarly when the L33 or L12 Improved leaders were employed in either EFS or huPGK promoter cassettes. Importantly, the ‘ON’ levels (no TRAP) for the new variants were similar to the non-overlapping tbs/Kozak variants, indicating that the Kozak sequences within the new variants were effective at directing efficient translation.

The data indicates that all of the tbs-Kozak variants were capable of similar levels of ‘ON’ transgene expression, showing that the Kozak sequences directed efficient levels of translation initiation. Variant 1 was poorly repressed by TRAP when compared to the original reporter configuration (10-fold higher levels of GFP expression in the presence of TRAP). However, surprisingly Variants 0, 2 and 3 were repressed to lower levels compared to the original configuration.

TABLE II
Optimal 3′tbs-Kozak junction sequences

| Panel I | Optimal 3′tbs-Kozak junction |
|---|---|
| Original | [kagnn]acaGCCACCATG |
| Variant ‘0’ | [kaGCC][GAGAT]G |
| Variant ‘1’ | [kagGC][GAGCA]TG |
| Variant ‘2’ | [kagnG][GAGCC]ATG |
| Variant ‘3’ | [kagnn][GAGAC]CATG |

[Panel I] Variant Kozak sequences designed to overlap the 3′ end of the upstream tbs sequence. The extended Kozak sequence was placed such that the main transgene ATG initiation codon is placed 9 nucleotides downstream of the 3′ terminal KAGNN repeat of the tbs i.e. there is no overlap. In order to position the main transgene ATG initiation codon closer to the upstream tbs, the four variants were designed such that the consensus KAGNN repeat of the 3′ terminal tbs repeats were maintained whilst also maintaining an efficient extended Kozak consensus sequence (herein defined as GNNRVVATG). The KAGNN repeat sequences are within brackets and the Kozak sequences in bold caps.

### Example 8: Incorporating Improved Overlapping Tbs-Kozak Variants into the Full Length, Intron-Containing EF1a Promoter

The previous Example indicated that the overlapping tbs-Kozak variants improved repression, compared to non-overlapping tbs/Kozak variants, and this was demonstrated in the context of the EFS promoter (the EF1a promoter truncated by removing its embedded intron). To assess if the tbs-Kozak variants ‘performed’ similarly within the full length EF1a promoter (i.e. post splicing-out of the intron), three tbs-Kozak variants (0, 2 and 3) were cloned into a GFP reporter cassette containing the EF1a promoter (FIG. 16A). After splicing, the 5′UTR contains exon1 (i.e. the L33 Improved leader), a short 12 nt sequence comprising the first nucleotides of exon2, and then a tbs-Kozak variant sequence (SEQ ID NO: 106). These GFP reporter plasmids, along with a reporter containing no tbs, were individually co-transfected into suspension, serum-free HEK293T cells +/−a TRAP-expression plasmid, and GFP expression measured two days post-transfection by flow cytometry. A GFP Expression Score (% GFP positive cells×MFI) was generated from flow cytometry data and plotted (FIG. 16B). Further, these tbs-containing GFP expression cassettes were cloned into an HIV-1 based lentiviral vector genome plasmid (in which the MSD and crSD were inactivated, see below), and a similar experiment carried out in suspension, serum-free HEK293T cells, resulting in GFP Expression scores (FIG. 16C). The data show that indeed the overlapping tbs-Kozak variants were capable of improved transgene repression compared to the non-overlapping tbs/Kozak variant employed.

### Example 9: Occlusion of Progressively More of the Core Kozak Sequence by the 3′ Terminal KAGNN Repeat of the Tbs Results in Progressively Greater Transgene Repression by TRAP

In Example 7, a limited number of overlapping tbs-Kozak variants were generated and tested in the context of non-intron-containing promoters EFS and huPGK. These variants were also then tested in the full EF1a promoter, which contains an intron in Example 5, showing that this difficult-to-repress promoter could be repressed by employing overlapping tbs-Kozak variants.

To further exemplify the principle of improved TRAP repression by ‘hiding’ the core Kozak sequence within the 3′ terminal KAGNN repeat of the tbs, a panel of new variants were designed (see Table IV). These were based on all the possible variants encoded by three ‘overlap groups’; KAGatg (where the KAGNN consensus overlaps the ATG of the core Kozak as much as possible, i.e. overlaps by the first two nucleotides of the ATG of the core Kozak), KAGNatg (where the KAGNN consensus overlaps the first nucleotide of the ATG of the core Kozak) and KAGNNatg (where the KAGNN consensus does not overlap the ATG of the core Kozak). (The initial variants tbs-kzkV0, tbs-kzkV1 and tbs-kzkV2 in Examples 3 and 5 fall within these defined groups). Any variants within these groups that generated a ‘GT’ dinucleotide were not generated/evaluated because of the undesirable possibility that these may produce cryptic splice donor sites.

TABLE III
Overlapping tbs-Kozak variants generated for
further exemplification
All the possible variants representing ′overlap
groups′ pertaining to the consensus of KAGatg or
KAGNatg or KAGNNatg were generated, except for
those resulting in a ′GT′ dinucleotide which might
generate an unwanted (cryptic) splice donor site.
The first 10 KAGNN repeats are presented as a
consensus here for clarity but were principally
the first 48 nucleotides of SEQ ID NO: 8. The 3′
terminal tbs KAGNN is presented (italicised and
bracketed) as encoded in each variant; the core
Kozak consensus is in bold and any nucleotides
presented as being part of the broader, extended
Kozak consensus are underlined.

| | | Overlapping tbs |
|---|---|---|
| Overlap | tbs-Kozak | 3′KAGNN-Kozak |
| Group | variant ID | variants |
| N/A | Non-overlapping | [KAGNN]x10-[KAGNN] |
| | tbs-Kozak | ACAGCCACCATG |
| KAGatg | tbskzkV0.G | [KAGNN]x10-[GAGAT]G |
| | tbskzkV0.T | [KAGNN]x10-[TAGAT]G |
| KAGNatg | tbskzkV1.0 | [KAGNN]x10-[G AGCA]TG |
| | tbskzkV1.1 | [KAGNN]x10-[G AGAA]TG |
| | tbskzkV1.2 | [KAGNN]x10-[G AGGA]TG |
| | tbskzkV1.3 | [KAGNN]x10-[T AGAA]TG |
| | tbskzkV1.4 | [KAGNN]x10-[T AGCA]TG |
| | tbskzkV1.5 | [KAGNN]x10-[T AGGA]TG |
| KAGNNatg | tbskzkV2.0 | [KAGNN]x10-[GA GCC]ATG |
| | tbskzkV2.1 | [KAGNN]x10-[GA GAA]ATG |
| | tbskzkV2.2 | [KAGNN]x10-[GA GAG]ATG |
| | tbskzkV2.3 | [KAGNN]x10-[GA GAC]ATG |
| | tbskzkV2.4 | [KAGNN]x10-[GA GAT]ATG |
| | tbskzkV2.5 | [KAGNN]x10-[GA GCA]ATG |
| | tbskzkV2.6 | [KAGNN]x10-[GA GCG]ATG |
| | tbskzkV2.7 | [KAGNN]x10-[GA GCT]ATG |
| | tbskzkV2.8 | [KAGNN]x10-[GA GGA]ATG |
| | tbskzkV2.9 | [KAGNN]x10-[GA GGG]ATG |
| | tbskzkV2.10 | [KAGNN]x10-[GA GGC]ATG |
| | tbskzkV2.11 | [KAGNN]x10-[TA GAA]ATG |
| | tbskzkV2.12 | [KAGNN]x10-[TA GAG]ATG |
| | tbskzkV2.13 | [KAGNN]x10-[TA GAC]ATG |
| | tbskzkV2.14 | [KAGNN]x10-[TA GAT]ATG |
| | tbskzkV2.15 | [KAGNN]x10-[TA GCA]ATG |
| | tbskzkV2.16 | [KAGNN]x10-[TA GCG]ATG |
| | tbskzkV2.17 | [KAGNN]x10-[TA GCC]ATG |
| | tbskzkV2.18 | [KAGNN]x10-[TA GCT]ATG |
| | tbskzkV2.19 | [KAGNN]x10-[TA GGA]ATG |
| | tbskzkV2.20 | [KAGNN]x10-[TA GGG]ATG |
| | tbskzkV2.21 | [KAGNN]x10-[TA GGC]ATG |

Generally, most of the variants conformed to the preferred core Kozak consensus of RVVATG, whilst simultaneously being restricted to containing the denoted KAGNN tbs consensus. These variants were cloned into a pEF1α-GFP reporter plasmid, and therefore the 5′UTR contained (after splicing of its intron) the L33 leader (exon 1) plus a short 12nt sequence from exon 2, which was previously shown in Example 5 to be less repressible by TRAP unless an overlapping tbs-Kozak variant was used. Suspension (serum-free) HEK293T cells were transfected with these variants individually with or without a TRAP-expression plasmid under conditions that typically reflected lentiviral vector (LV) transfection/production (e.g. inclusion of sodium butyrate induction), and flow cytometry performed at typical LV harvest times (2 days post-transfection). Global GFP Expression scores were generated (% GFP×MFI; ArbU) for +/−TRAP conditions and then fold-repression values generated and plotted in FIG. 17A. The results demonstrate that the more the core Kozak consensus sequence overlaps the 3′ terminal KAGNN tbs repeat, the better the level of TRAP repression. Statistical analysis (T-Test) of the fold differences of each overlap group demonstrate a significant increase in repression as more of the core Kozak was overlapped with the 3′ terminal KAGNN tbs repeat, with the best repression scores coming from the KAGatg group where ⅔^{rd} of the initiation codon forms part of the KAGNN repeat. All overlap variants produced statistically greater transgene repression by TRAP compared to the non-overlapping tbs variant.

The data were further stratified in FIG. 17B, where non-repressed ‘ON’ transgene levels were displayed from highest to lowest. The two variants from the KAGatg overlap group are highlighted to show that for these two best performing variants in terms of TRAP repression, the GAGatg variant (tbskzkV0.G) gave the highest ‘ON’ levels, presumably because it conforms to the core Kozak consensus of RVVATG, whereas TAGatg (tbskzkV0.T) does not.

All these data support the general principle that overlapping tbs-Kozak variants are more effective in mediating TRAP repression compared to non-overlapping tbs variants, and that preferably the tbs-Kozak overlap conforms to the core Kozak consensus of RVVATG to ensure good ‘ON’ transgene expression in vector-transduced target cells. Thus, the employment of these novel tbs-Kozak variants will enable more efficient transgene repression during viral vector production, potentially leading to increase in viral vector titres if said transgene protein activity is detrimental to viral vector titres and/or activity.

### Example 10: Further Use of an Optimal Overlapping Tbs-Kozak Variant to Improve TRAP-Mediated Repression of Common Promoters Harbouring an Intron

In Example 8, the use of overlapping tbs-Kozak variants was shown to improve TRAP-mediated repression when using the full length EF1a promoter, which contains an intron. For similar promoters used widely in viral vector genomes for gene therapy—for example the CAG promoter—the presence of embedded exon/intron sequence means that the degree of TRAP-mediated repression may be affected by sequences within ‘native’ exonic sequences. From the point of view of improving TRAP-mediated repression, it may not be obvious or feasible to alter exonic sequences, especially if these are involved in splicing enhancement (e.g. a splice enhancer element close to the splice donor site). The widely used CAG promoter comprises the CMV enhancer element, the core chicken β-Actin gene promoter-exon1-intron sequence, and the splice acceptor-exonic sequence from the rabbit β-Globin gene. Elsewhere in the invention it was surprisingly found that exon 1 from the EF1a promoter (L33) could be used upstream of all types of tbs variants to improve TRAP-mediated repression, presumably providing good sequence context to support formation a stable TRAP-tbs complex, to enable efficient translation inhibition. The overlapping tbs-Kozak variants were shown to aid TRAP-mediated repression in both EF1a (intron-containing) and several other promoters lacking introns.

In this example (see FIG. 18A), both features were applied to improving TRAP-mediated repression from the CAG promoter by [a] placing the tbskzkV0.G variant (also referred to as Variant ‘0’ in other Examples) within the ‘native’ 5′UTR region of the CAG promoter (SEQ ID NO: 117) and [b], swapping the entire ‘native’ intron-containing 5′UTR region with the EF1a 5′UTR-intron region harbouring the tbskzkV0.G variant (SEQ ID NO: 118). The corresponding spliced sequences are shown as SEQ ID NO: 119 and SEQ ID NO: 120, respectively. In addition, it was shown by exemplification that a promoter typically used without an intron in viral vector genomes—in this case CMV—could be appended with an artificial 5′UTR containing a heterologous intron, expression of which had previously shown to be efficiently repressed by TRAP. Specifically, the 5′UTR with the EF1a 5′UTR-intron region harbouring the tbskzkV0.G variant (SEQ ID NO: 118) was used in the CMV promoter context.

These reporter constructs (encoding GFP) were evaluated for both ‘ON’ expression levels and TRAP-mediated repression in suspension (serum-free) HEK293T cells, modelling a viral vector production scenario. Cells were transfected with GFP reporter plasmid +/−pTRAP, cultures induced with sodium butyrate after transfection (as per typical viral vector production) and cells analysed for GFP expression ˜2 days post-transfection (i.e. at typical viral vector harvest point). GFP Expression scores (% GFP positive×MFI; ArbU) were generated and plotted (FIG. 18B), and TRAP-repression scores displayed. These data indicate that TRAP-mediated expression in typical viral vector production cells can be improved from the CAG promoter using the tbskzkV0.G variant, from 3-fold to 30-fold. Moreover, the data show that the ‘native’ 5′UTR region sequence from different promoters can be replaced with the intron-containing EF1a 5′UTR harbouring the tbsKzkV0.G variant, leading to both substantially improved TRAP-mediated repression (30-40 fold to >100-fold) and maintenance of high gene expression in the absence of TRAP (i.e. modelling expression in viral vector-transduced target cells). Thus, the novel EF1a-5′UTR-intron-tbskzkV0.G sequence may be useful in providing heterologous promoters the known benefits imparted by an intron in target cells (i.e. increased gene expression), whilst also enabling efficient repression of the transgene protein during viral vector production, potentially leading to increase in viral vector titres if said transgene protein activity is detrimental to viral vector titres. This also applies to the use of the EF1a-5′UTR-intron sequence with other overlapping tbs-Kozak sequences.

### Example 11—Evaluation of the Impact of Mutation of the Major Splice Donor Alone, or in Combination with Additional Mutation of the Adjacent Cryptic Splice Donor Site, on Aberrant Splice, Vectors Titres and Response to Modified U1 snRNA

To assess the impact of the major splice donor site mutation alone or in combination with the cryptic splice donor (crSD; now also referred to as ‘crSD1’) mutation (present in MSD-2KO), another variant was cloned—named ‘MSD-1KO’ (FIG. 20A). This variant harboured only the GT>CA mutation in the MSD site. The MSD-1KO variant genome, alongside a standard LV genome and the MSD-2KO genome (all containing an EF1a-GFP transgene cassette) were used to produce LV-GFP crude harvest material by transient transfection of suspension (serum-free) HEK293T cells, with or without modified U1 snRNA targeted to the packaging region of the vRNA (256U1). LV-GFP titres are displayed in FIG. 20D, and show that mutation in the MSD site alone is sufficient to reduce LV titres, and that these titres are recoverable to the same level as observed for the MSD-2KO vector. Post-production cell analysis of aberrant splicing from the MSD region within the SL2 loop was performed on extracted, polyA-selected mRNA by RT-PCR using primers that allowed detection of both unspliced or aberrantly spliced products (FIG. 20C; see FIG. 23 for position of primers). The data show that mutation of the MSD alone activates the cryptic splice donor (crSD[1]) immediately downstream. The analysis also revealed that aberrant splicing from the MSD region in SL2 is fully ablated by mutation of both the MSD and adjacent cryptic splice donor (crSD[1]) present in the ‘MSD-2KO’ variant but that this activated another minor splice donor site present within the SL4 loop (crSD2) further downstream in the packaging sequence. (This was also evident in a previous example—see FIG. 4Bii).

### Example 12—Evaluation of the Impact of Mutation of Cryptic Splice Donors in the SL4 Loop of the Packaging Sequence in Combination with Mutation of the Major Splice Donor and Cryptic Splice Donor Sites in SL2

In Example 11 it was shown that mutation of both the MSD and crSD1 completely eliminated aberrant splicing from the SL2 region of the packaging signal but that this activated a further cryptic splice donor in the SL4 loop (see FIG. 20A; ‘crSD2’). To assess if the crSD2 site in SL4 could be mutated to abolish activity—and being mindful that further modifications to the packaging sequence may compromise the folding of RNA required for efficient packaging—MSD-3KO_1 and MSD-3KO_2 variant were designed. These contained the MSD-2KO mutations in SL2, and a single nucleotide mutation of the ‘GT’ dinucleotide in the crSD2 site (see FIG. 20A). MSD-3KO_1 mutated the G of the GT dinucleotide to a ‘C’, whereas the MSD-3KO_2 mutation was a T>C mutation of the GT dinucleotide. Note that the T>C mutation predicts better base-pairing in the SL4 loop, and also in the tertiary model of the broader packaging sequence (Keane et al. Science. 2015 May 22; 348(6237): 917-921)). LV-EF1a-GFP vectors containing these modifications were produced as described in Example 11, including analysis of vector RNA from post-production cells (FIG. 21A) and titration of resulting vector harvests (FIG. 21B). Aberrant splicing in the MSD-3KO_1 variant did not appear to be ablated and on sequencing of the exon-exon junction of the RT-PCR product it was found that this product was derived from another cryptic splice donor site (crSD3) was activated 10 nucleotides downstream (see FIG. 21A lanes 7 and 8, and FIG. 20A for sequence of crSD3). Construction and testing of MSD-4KO_1 and MSD-4KO_2 demonstrated that aberrant splicing from crSD3 was indeed activated by the G>C mutation in crSD2, since this was abolished with mutation of crSD3 (FIG. 21A; lanes 11 to 14). However, surprisingly for the MSD-3KO_2 variant, not only was aberrant splicing from crSD2 abolished but the crSD3 site was not activated (FIG. 21A; lanes 9 and 10), indicating that a cryptic splicing enhancer for crSD3 had been also abolished by the single T>C mutation of MSD-3KO_2 in the SL4 loop.

More surprisingly was the impact of the MSD-2KOm5 mutation on cryptic splicing from crSD2 or crSD3. In previous Examples it was shown that the MSD-2KOm5 variant is less attenuated than other MSD-2KO splice donor mutants, and this could lead to further benefits in titre recovery by modified U1 snRNA. Without wishing to be bound by theory, the MSD-2KOm5 variant was designed such that maximal annealing with endogenous U1 snRNA might occur (without being a functional splice donor) based on the hypothesis that recruitment of U1 snRNA by vRNA might be beneficial for stability (separately and additionally to the use of modified U1 snRNA targeted to the SL1 loop). FIG. 20B displays how standard, MSD-2KO and MSD-2KOm5 vector genome vRNAs are predicted to anneal with endogenous U1 snRNA, despite the mutated genomes not containing splice donor sites. The MSD-2KOm5 variant in theory can recruit endogenous U1 snRNA with more stability (i.e. greater number of hydrogen bonds partaking in base-pairing) than the MSD-2KO variant, and perhaps to a greater extent than standard LV genomes, but crucially without leading to splicing events. Additionally, MSD-2KOm5 was designed to ensure a stable SL2 loop could form, as this may be important for correct folding of the packaging sequence. When performing the same analyses for the MSD-2KOm5 (and related MSD-3KO_2 and MSD-4KO_2 variants), it was surprisingly found that no aberrant splicing occurred from crSD2 or crSD3 within SL4. Again, without being bound by theory, it is proposed that the proximity of recruitment of endogenous U1 snRNA to the SL2 loop may impede the recognition of cryptic splice sites in the SL4 loop.

All the splice donor mutant vectors produced in this further study had reduced titres (with MSD-2KOm5 being the least attenuated) but all were substantially boosted by the supply of modified U1 snRNA in trans (FIG. 21B).

Altogether, this indicates that minor mutations leading to functional ablation of both the MSD and crSD1 sites lead to a robust reduction in general aberrant splicing from the packaging sequence but that auxiliary mutations in the crSD2/3 sites of the SL4 loop may be required to eliminate it entirely. The preferred modification, leading to functional ablation of aberrant splicing from the MSD and crSD1 sites, is the more substantial modification as described by the MSD-2KOm5 variant, as this also has the surprising effect of not allowing activation of minor cryptic splice donor sites in the downstream SL4 loop, and vectors harbouring this modification can be made to the highest titres in the presence of modified U1 snRNA targeted to its packaging region.

### Example 13—Splice Donor Mutated LV Genomes Result in Fewer Transcriptional ‘Read-Through’ Events by Upstream Cellular Promoters in Target Cells

Lentiviral vector integration into target cells is semi-random, with LVs showing preference for integration into transcriptionally active cellular genes. It has been shown by others that transcriptional read-through or ‘read-in’ to integrated LVs from upstream cellular promoters can occur, and mobilise/interact with sequences within the LV genome (see e.g. Moiani et al. J Clin Invest. 2012 May 1; 122(5): 1653-1666). For the present invention it is anticipated that a further benefit of MSD-mutated LVs is improved patient safety regarding the effects of integrated LV within the chromosome of target cells. Since it has been demonstrated that for wild type HIV-1, recruitment of endogenous U1 snRNA to the major splice donor result in suppression of the 5′LTR polyadenylation signal, a similar mechanism occurring in standard LVs during read-in could conceivably exacerbate this due to recruitment of U1 snRNA. Therefore ablation of the MSD (and cryptic SDs) within the LV packaging signal may lead to reduced transcriptional read-in due to increased use of the 5′-SINLTR encoded polyA signal (see FIG. 22).

To test this, the MSD-mutated LV vectors and standard LV vectors produced in Example 12 (see FIG. 21B) were used to transduce HEK293T cells and 92BR primary cells (Donkey fibroblast) at matched MOIs (only MSD-mutated LV vector preps made in the presence of 256U1 were used since these had similar titres to standard vector preps). Transduced cell cultures were passaged for 10 days to allow loss of both unintegrated LV cDNA, and the potential RNA generated from them, Cell RNA was then extracted, DNAse-treated and subjected to RT-qPCR analysis using primers to the packaging region of the LVs (see FIG. 22 for positions of primers). Copies of detected HIV packaging RNA were normalised to GAPDH RNA signal (RNA loading control) and then for integrated LV DNA copy-number. Total normalised copies of detected HIV packaging RNA for the standard LV vector (made without 256U1) was set to 1 for each cell type, and all other relevant normalised copy-numbers set relative to this. FIG. 23 displays the results of this analysis, indicating that indeed the feature of having mutated MSD and cryptic SDs within the LV genome led to between 2- and 5-fold reduction in transcriptional read-in events. All these data indicate that MSD/crSD mutated LVs produce a better safety profile to standard LVs in transduced cells, with less propensity to allow for LV backbone mobilisation and/or interaction with LV sequences.

1-54. (canceled)

55. A nucleic acid encoding an RNA genome of a lentiviral vector, wherein the RNA genome comprises an inactivated major splice donor site and an inactivated cryptic splice donor site 3′ to the major splice donor site, wherein the nucleic acid comprises a nucleotide sequence set forth in SEQ ID NO: 7, SEQ ID NO: 14, or SEQ ID NO: 217.

56. The nucleic acid according to claim 55, comprising (i) a nucleotide sequence set forth in SEQ ID NO: 14, and (ii) a nucleotide sequence set forth in SEQ ID NO: 228 or SEQ ID NO: 232.

57. The nucleic acid according to claim 55, further comprising a nucleic acid of interest.

58. The nucleic acid according to claim 55, wherein the nucleic acid does not comprise a U3 promoter.

59. The nucleic acid according to claim 55, comprising a self-inactivating LTR.

60. The nucleic acid according to claim 55, comprising a nucleotide sequence encoding a modified U1 snRNA, wherein the modified U1 snRNA is modified to bind to a nucleotide sequence within the packaging region of the RNA genome.

61. The nucleic acid according to claim 55, comprising a tryptophan RNA-binding attenuation protein (TRAP) binding site.

62. The nucleic acid according to claim 55, comprising a Kozak sequence, wherein the TRAP binding site overlaps the Kozak sequence.

63. An expression cassette comprising the nucleic acid according to claim 55.

64. A lentiviral vector production system comprising:
(i) the nucleic acid according to claim 55, wherein the RNA genome comprises a self-inactivating LTR, and wherein the RNA genome does not comprise a U3 promoter; and
(ii) nucleotide sequences encoding gag-pol, env, and, optionally, rev;
wherein the lentiviral vector production system does not comprise tat.

65. The lentiviral vector production system according to claim 64, further comprising a nucleotide sequence encoding a modified U1 snRNA, wherein the modified U1 snRNA is modified to bind to a nucleotide sequence within the packaging region of the RNA genome.

66. A cell comprising the nucleic acid according to claim 55.

67. A cell comprising the expression cassette according to claim 63.

68. A cell comprising the lentiviral vector production system according to claim 64.

69. A method for producing a lentiviral vector, the method comprising introducing into a cell the viral vector production system according to claim 64.

70. A nucleic acid encoding an RNA genome of a lentiviral vector, wherein the RNA genome comprises an inactivated major splice donor site and an inactivated cryptic splice donor site 3′ to the major splice donor site, wherein the nucleic acid comprises:
(i) a nucleotide sequence set forth in SEQ ID NO: 5 and a nucleotide sequence set forth in SEQ ID NO: 226;
(ii) a nucleotide sequence set forth in SEQ ID NO: 5 and a nucleotide sequence set forth in SEQ ID NO: 228;
(iii) a nucleotide sequence set forth in SEQ ID NO: 5 and a nucleotide sequence set forth in SEQ ID NO: 230; or
(iv) a nucleotide sequence set forth in SEQ ID NO:5 and a nucleotide sequence set forth in SEQ ID NO:232.

71. The nucleic acid according to claim 70, further comprising a nucleic acid of interest.

72. The nucleic acid according to claim 70, wherein the nucleic acid does not comprise a U3 promoter.

73. The nucleic acid according to claim 70, comprising a self-inactivating LTR.

74. The nucleic acid according to claim 70, comprising a nucleotide sequence encoding a modified U1 snRNA, wherein the modified U1 snRNA is modified to bind to a nucleotide sequence within the packaging region of the RNA genome.

75. The nucleic acid according to claim 70, comprising a tryptophan RNA-binding attenuation protein (TRAP) binding site.

76. The nucleic acid according to claim 70, comprising a Kozak sequence, wherein the TRAP binding site overlaps the Kozak sequence.

77. An expression cassette comprising the nucleic acid according to claim 70.

78. A lentiviral vector production system comprising:
(i) the nucleic acid according to claim 70, wherein the RNA genome comprises a self-inactivating LTR, and wherein the RNA genome does not comprise a U3 promoter; and
(ii) nucleotide sequences encoding gag-pol, env, and, optionally, rev;
wherein the lentiviral vector production system does not comprise tat.

79. The lentiviral vector production system according to claim 78, further comprising a nucleotide sequence encoding a modified U1 snRNA, wherein the modified U1 snRNA is modified to bind to a nucleotide sequence within the packaging region of the RNA genome.

80. A cell comprising the nucleic acid according to claim 70.

81. A cell comprising the expression cassette according to claim 77.

82. A cell comprising the lentiviral vector production system according to claim 78.

83. A method for producing a lentiviral vector, the method comprising introducing into a cell the viral vector production system according to claim 78.

84. A lentiviral vector production system comprising:
(i) a nucleic acid encoding an RNA genome of a lentiviral vector, wherein the RNA genome comprises an inactivated major splice donor site and an inactivated cryptic splice donor site 3′ to the major splice donor site, wherein the RNA genome comprises a self-inactivating LTR, and wherein the RNA genome does not comprise a U3 promoter;
(ii) nucleotide sequences encoding gag-pol, env, and, optionally, rev; and
(iii) a nucleotide sequence encoding a modified U1 snRNA, wherein the modified U1 snRNA is modified to bind to a nucleotide sequence within the packaging region of the RNA genome;
wherein the lentiviral vector production system does not comprise tat.

85. A cell comprising the lentiviral vector production system according to claim 84.

86. A method for producing a lentiviral vector, the method comprising introducing into a cell the viral vector production system according to claim 84.
